(19) United States
(12) Patent Application Publication
(10) Pub. No.: US 2016/0331686 A1
(43) Pub. Date: Nov. 17, 2016

(54) Compositions and Methods for Yeast Extracellular Vesicles as Delivery Systems

(71) Applicant: CLSN Laboratories, Inc., Wilmington, DE (US)

(72) Inventors: Kevin J. POLACH, Lawrenceville, NJ (US); Daniel W. Neef, Lawrenceville, NJ (US); Jason G. Fewell, Lawrenceville, NJ (US); Khursheed Anwer, Lawrenceville, NJ (US)

(73) Assignee: CLSN Laboratories, Inc., Wilmington, DE (US)

(21) Appl. No.: 15/152,170

(22) Filed: May 11, 2016

Related U.S. Application Data
(60) Provisional application No. 62/160,452, filed on May 12, 2015.

(51) Int. Cl. A61K 9/127; A61K 9/00; A61K 48/00; A61K 47/48; A61K 38/46; C07K 16/18; C12N 15/113; C12N 15/81; A61K 38/02

(52) U.S. Cl. CPC A61K 9/127; C12N 15/815; A61K 9/0019; A61K 48/0008; A61K 48/0075; A61K 38/02; A61K 38/465; C12Y 301/00; C07K 16/18; C12N 15/113; A61K 47/48815; C07K 2317/80; C12N 2310/141; C12N 2320/32

(57) ABSTRACT

The present invention provides compositions of yeast extracellular vesicles comprising biologically active molecules, methods for making the same, and methods for the use of the yeast extracellular vesicles to deliver biologically active molecules to target cells. In addition, the invention provides cells and compositions comprising the biologically active molecules and vesicles, which can be used as transfection reagents. The invention further provides methods for producing said compositions of biologically active molecules with vesicles as well as the cells that produce those compositions. Compositions and methods for delivering biologically active molecules, such as a small molecule, a DNA expression plasmid, an RNA molecule, a peptide, or a protein, to cells and/or tissues are provided. The compositions and cells are useful, for example, in delivering biologically active RNA molecules to cells to modulate target gene expression in the diagnosis, prevention, amelioration, and/or treatment of diseases, disorders, or conditions in a subject or organism.

### CROSS-REFERENCE TO RELATED APPLICATIONS

This application claims the benefit of U.S. Application No. 62/160,452, filed May 12, 2015, the entire contents of which are hereby incorporated by reference.

### REFERENCE TO SEQUENCE LISTING SUBMITTED ELECTRONICALLY

The content of the electronically submitted sequence listing (File Name: 2437_0460001_SeqListing.txt; Size: 28,802 bytes; and Date of Creation: May 10, 2016), filed herewith, is incorporated by reference in its entirety.

### FIELD OF THE INVENTION

This invention relates to compositions, methods and processes for delivery of biologically active molecules.

### BACKGROUND OF THE INVENTION

The delivery of biologically active macromolecules to cells and tissues in vivo remains a challenge to the development of new biological drugs [1-4]. Synthetic delivery vehicles now include a wide array of molecules and macromolecular assemblies including proteins, nucleic acids, polymers and lipid vesicles. Each of these reagents must possess attributes that are required to facilitate safe and efficient delivery of biological drugs including: 1) the capacity to interact with the drug to be delivered, either through direct binding or some mode of trapping or encapsulation, 2) a mechanism for delivering that drug to the necessary site of action, and 3) an acceptable level of toxicity and immune response in the treated cell or organism. Though interaction between the drug and reagent can be relatively straightforward, inefficient delivery to the target site (intracellular or extracellular) and/or prohibitively high toxicity/immunogenicity can create a limited therapeutic window of acceptable drug concentrations that can be used in the treatment of disease. Most research efforts related to drug delivery systems are designed to widen the therapeutic window by increasing delivery efficiency and decreasing toxic/immune responses, allowing for a broader range of useful drug doses [5]. These approaches have led to various modifications of delivery systems, including PEG-modifications that improve drug circulation times while decreasing toxicity and targeting ligands that allow for drug delivery preferentially to target cells, such as cancer cells. However, the synthetic delivery systems remain less than ideal in reproducing the natural mechanisms of safe and efficient loading and translocation of biological agents.

Naturally occurring lipid vesicles are produced by a wide array of cell types with biological complexity ranging from bacteria to mammals [6-9]. Vesicles may serve as a non-classical transport network, in a paracrine (and possibly endocrine) fashion [10]. A number of significant technical hurdles limit application of naturally derived vesicles as delivery vehicles, even as simple transfection reagents. These include the lack of an effective mechanism for loading vesicles with a biologically active molecule of interest and subsequent purification of those loaded vesicles in quantities sufficient for use [11, 12]. Reports have described the collection of mammalian exosomes and subsequent loading of those exosomes with exogenous RNA using chemical or electro-mechanical methodologies for delivery into target cells [12-17]. However, the culture density that can be achieved with mammalian cell cultures limits large-scale production and subsequent practical applications of these systems.

Yeast-derived vesicles were discussed in a recent perspective paper published in Drug Discovery Today [18]. As noted in the paper, no study had demonstrated the use of yeast secretory vesicles for carriers in human therapeutic applications. In this paper, the authors speculated on the potential application of post-Golgi vesicles (PGVs) for use in the biomedical field. PGVs are typically transient in nature, shuttling cargo from the Golgi complex to the extracellular surface. Thus, the continuous secretion in wild type strains of S. cerevisiae prevent intracellular build-up of PGVs (i.e., the low number makes recover difficult). The authors propose using sec mutant S. cerevisiae strains to allow accumulation of PGVs. The use of signal peptides to generate fusion tags with therapeutic proteins, to place the protein inside a PGV to possibly circumvent external procedures for incorporation of therapeutic proteins into PGVs is also proposed. However, successful use of mutant yeast cells to produce vesicles for therapeutic use was not disclosed.

### SUMMARY OF THE INVENTION

The present invention provides compositions and methods for the use of yeast extracellular vesicles to deliver biologically active molecules to target cells. In some embodiments, the invention provides compositions comprising biologically active molecules and yeast vesicles that are useful for the delivery of the biologically active molecules to extracellular spaces or to target cells. In addition, the invention provides cells and compositions comprising the biologically active molecules and vesicles, which can be used as transfection reagents. The invention further provides methods for producing said compositions comprising biologically active molecules within vesicles as well as the cells that produce the vesicles. Additionally, compositions and methods for delivering biologically active molecules (such as RNA, DNA, peptides or proteins) to cells and/or tissues are provided. The compositions and cells are useful, for example, in delivering biologically active RNA molecules to cells to modulate target gene expression in the diagnosis, prevention, amelioration, and/or treatment of diseases, disorders, or conditions in a subject or organism or as transfection reagents.

The present invention also provides methods for the delivery of bio-macromolecules utilizing lipid vesicles derived from yeast cells. Extracellular vesicles pre-loaded with a biological agent by the yeast cells can be collected from the growth media and purified for use as a drug delivery system for mammalian target cells. One approach involves the use of yeast cells transformed with a heterogenous DNA plasmid expressing a biologically active RNA molecule and producing extracellular vesicles loaded with that RNA molecule. The biologically active RNA molecule can be, e.g., a ribozyme, an antisense nucleic acid, an aptamer, a short interfering RNA (siRNA), a double-stranded RNA (dsRNA), a micro-RNA (miRNA), and a short hairpin RNA (shRNA) molecule, as well as mRNA transcripts encoding one or more biologically active peptides or proteins. These RNAs can have either a linear or circular form. In some embodiments, the circular RNAs can also include miRNA sponges. A vesicle-producing yeast cell can be generated by administering to a yeast cell one or more expression vectors designed to produce at least one biologically active RNA molecule. These RNA molecules are expressed in the nucleus of the yeast cell and delivered to the cytoplasm through endogenous nuclear export machinery, where the RNA molecules are incorporated into yeast extracellular vesicles, e.g., through sampling of the cytoplasm during vesicle formation. These vesicles accumulate in the growth media, allowing for separation from the yeast cells, purification and use as a delivery reagent (FIG. 1).

Certain aspects of the invention are directed to an extracellular vesicle comprising a vesicle membrane and a biologically active molecule, wherein the vesicle is derived from a yeast cell transformed with a polynucleotide which expresses or encodes the biologically active molecule and does not comprise a secretory domain sequence.

Certain aspects of the invention are directed to an extracellular vesicle comprising a vesicle membrane and a biologically active molecule which does not comprise a secretory domain, wherein the vesicle is derived from a yeast cell and the biologically active molecule is produced the yeast cell.

In some embodiments, the polynucleotide is a circular or linear DNA.

In some embodiments, the vesicle membrane further comprises a targeting peptide. In some embodiments, the targeting peptide is selected from a one or more targeting peptides listed in Table 5.

In some embodiments, the vesicle membrane further comprises an immune masking protein. In some embodiments, the immune masking protein is selected from a one or more immune masking proteins listed in Table 4.

In some embodiments, the vesicle membrane further comprises a CRISPR Cas9 protein and a crRNA guide sequence.

In some embodiments, the extracellular vesicle further comprise at least one endogenous yeast protein selected from the group consisting of SEC14, TSA1, GAS1, or any combination thereof.

In some embodiments, the extracellular vesicle is purified.

In some embodiments, the biologically active molecule is a DNA, a RNA, a peptide, or a protein.

In some embodiments, the RNA is mRNA, siRNA, RNAi, shRNA, miRNA, RNA ribozyme or RNA aptamer.

In some embodiments, the protein is an intrabody.

In some embodiments, the yeast cell is a non-pathogenic yeast strain.

In some embodiments, the yeast cell is a commensal yeast strain. In some embodiments, the yeast strain is Candida glabrala. In some embodiments, the yeast strain is Saccharomyces cerevisiae. In some embodiments, the yeast strain is Pichia pasloris. In some embodiments, the yeast strain is Kluyveromyces lactis.

In some embodiments, the cell wall biosynthesis enzyme chitin synthase 3 has been mutated or deleted from the yeast cell. In some embodiments, the cell wall biosynthesis enzyme chitin synthase 3 has been mutated or deleted from the Saccharomyces cerevisiae yeast cell.

Certain aspects of the invention are directed to an extracellular vesicle derived from yeast comprising a vesicle membrane and an endogenously produced biologically active molecule which does not comprise a secretory domain sequence, wherein the vesicle membrane comprises a transmembrane protein that functions as a targeting ligand for delivering the biologically active molecule to a mammalian target cell through interaction with a target cell receptor.

Certain aspects of the invention are directed to a method for producing an extracellular vesicle of the invention comprising transforming the yeast cells with an expression vector comprising a first polynucleotide and a second polynucleotide, wherein the first polynucleotide encodes a yeast origin of replication and the second polynucleotide encodes a transmembrane targeting ligand.

Certain aspects of the invention are directed to a yeast cell comprising an extracellular vesicles of the invention.

Certain aspects of the invention are directed to a yeast autonomous cytoplasmic linear expression vector comprising a first polynucleotide, a second polynucleotide, a third polynucleotide and a fourth polynucleotide, wherein the first polynucleotide encodes for a yeast origin of replication, the second polynucleotide encodes for an auxotrophic selectable marker, the third polynucleotide encodes for a mammalian nuclear localization signal, and the fourth polynucleotide encodes for a therapeutic RNA.

Certain aspects of the invention are directed to a yeast autonomous cytoplasmic linear expression vector comprising a first polynucleotide, a second polynucleotide, a third polynucleotide and a fourth polynucleotide, wherein the first polynucleotide encodes for a yeast origin of replication, the second polynucleotide encodes for an auxotrophic selectable marker, the third polynucleotide encodes for a mammalian nuclear localization signal, and the fourth polynucleotide encodes for a therapeutic polypeptide.

In some embodiments, the second polynucleotide encoding for an auxotrophic selectable marker further comprises a yeast promoter.

In some embodiments, the third polynucleotide encoding for a mammalian nuclear localization signal further comprises a mammalian promoter.

Certain aspects of the invention are directed to a method for purifying extracellular vesicles comprising a biologically active molecule comprising: a) transforming a yeast cell with an expression vector which expresses or encodes a biologically active molecule and does not comprise a secretory domain sequence, b) culturing the yeast cell in a growth media under conditions where the vesicles are released into the extracellular growth media, c) removing the yeast cells from the growth media, and d) purifying the vesicles from the growth media. In some embodiments, the method of purifying further comprising precipitating the vesicles with polyethylene glycol (peg) after step (c). In some embodiments, the vesicles are purified in the void volume of a size exclusion chromatography column. In some embodiments, the vesicles are purified by affinity chromatography.

In some embodiments, the biologically active molecule is the expression vector, a therapeutic yeast autonomous cytoplasmic linear plasmid comprising a first polynucleotide, a second polynucleotide, and a third polynucleotide, wherein the first polynucleotide encodes for a yeast origin of replication, the second polynucleotide encodes for a mammalian nuclear localization signal, and the third polynucleotide encodes a therapeutic polypeptide, the expression of which is driven by a mammalian promoter.

In some embodiments, the biologically active molecule is a therapeutic RNA transcribed from the expression vector comprising a first polynucleotide and a second polynucleotide, wherein the first polynucleotide encodes for a yeast origin of replication and the second polynucleotide encodes a biologically active RNA sequence, the expression of which is driven by a yeast promoter.

In some embodiments, the biologically active molecule is a therapeutic polypeptide encoded by the expression vector comprising a first polynucleotide and a second polynucleotide, wherein the first polynucleotide encodes for a yeast origin of replication and the second polynucleotide encodes an mRNA sequence encoding a therapeutic polypeptide, consisting of a vesicle targeting polypeptide domain and a therapeutic polypeptide domain, with the mRNA expression driven by a yeast promoter.

Certain aspects of the invention are directed to a method for delivering a yeast derived extracellular vesicle comprising a biologically active molecule to mammalian target cells in vitro comprising adding the vesicles to the growth media of the target cells under conditions where the vesicles can be taken up through fusion with the cell membrane or endocytosis, resulting in transfer of the biologically active molecule to the target cell.

Certain aspects of the invention are directed to a method for delivering a yeast derived extracellular vesicle comprising a biologically active molecule to mammalian target cells in vivo comprising administering the vesicles to a subject under conditions where the vesicles can be taken up by target cells through fusion with the cell membrane or endocytosis, resulting in transfer of the biologically active molecule to the target cell.

In some embodiments, the vesicles are administered to the subject by local or systemic injection

### BRIEF DESCRIPTION OF THE DRAWINGS

FIG. 1. Schematic for vesicle production and purification process. Yeast cultures are grown to a level of confluence optimized for extracellular vesicle production. Cells are removed from the growth media by centrifugation and filtering to yield a media fraction containing extracellular vesicles and free extracellular protein. The extracellular vesicles and protein are then precipitated from the media with polyethylene glycol (PEG, MWt optimized for vesicle precipitation) and resuspended in a small volume of PBS. This mixture is then run over a size exclusion column (equilibrated in PBS) to isolate vesicles moving in the void volume from the free protein and PEG molecules, which are retained by the resin. The purified vesicles can then be calculated from particle counting experiments, or absorbance readings at 260 nm for nucleic acid or 280 nm for protein.

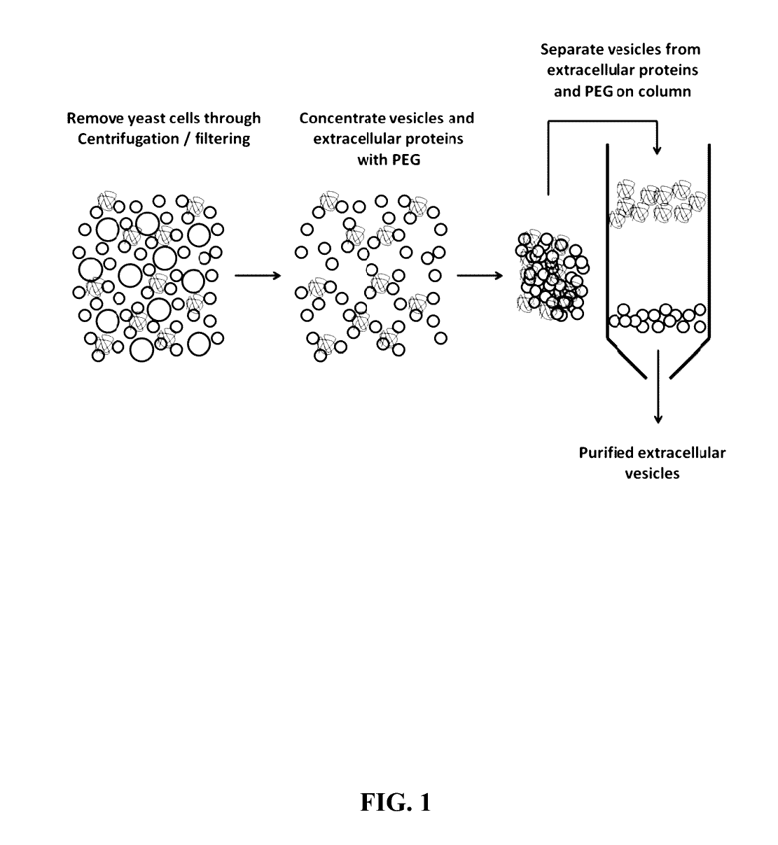

FIG. 2. Schematic for delivery of a therapeutic RNA by yeast vesicles. Yeast cells are transformed with a circular DNA plasmid encoding the biologically active RNA of interest (yeast-specific promoters) and cultures are grown to a level of confluence optimized for extracellular vesicle production. The RNA is expressed in the nucleus of the yeast cell, exported to the cytoplasm and taken into yeast extracellular vesicles through cytoplasmic sampling. Cells are removed from the growth media by centrifugation and filtering to yield a media fraction containing extracellular vesicles and free extracellular protein. The extracellular vesicles and protein are then precipitated from the media with polyethylene glycol (PEG, MWt optimized for vesicle precipitation) and resuspended in a small volume of PBS. This mixture is then run over a size exclusion column (equilibrated in PBS) to isolate vesicles moving in the void volume from the free protein and PEG molecules, which are retained by the resin. The concentration of purified vesicles can then be determined from particle counting experiments, or absorbance readings at 260 nm for nucleic acid or 280 nm for protein. The purified vesicles are then added to mammalian target cells, which take up the vesicles and the biologically active RNA. Release of the RNA to the cytoplasm of the target cell then allows for biological activity.

FIG. 3. Schematic for delivery of a yeast autonomous cytoplasmic linear expression plasmid by yeast vesicles. Yeast cells are transformed with an autonomously replicating cytoplasmic linear DNA plasmid encoding the biologically active molecule of interest (RNA or protein) and cultures are grown to a level of confluence optimized for extracellular vesicle production. Cells are removed from the growth media by centrifugation and filtering to yield a media fraction containing extracellular vesicles and free extracellular protein. The extracellular vesicles and protein are then precipitated from the media and purified on a size exclusion column as described in FIG. 1. The concentration of the purified vesicles can then be determined from particle counting experiments, or absorbance readings at 260 nm for nucleic acid or 280 nm for protein. The purified vesicles are then added to mammalian target cells, which take up the vesicles and the cytoplasmic linear plasmid. Linear plasmids may be expressed in the cytoplasm of mammalian target cells through factors carried by and expressed from the plasmid (plasmid-specific promoters) or shuttled to the nucleus via a nuclear localization signal (NLS) for expression by pathways endogenous to the target cell (cell-specific promoters).

FIG. 4. Schematic for delivery of a therapeutic polypeptide by yeast vesicles. Yeast cells are transformed with a circular DNA plasmid encoding the biologically active protein of interest (yeast-specific promoters) and cultures are grown to a level of confluence optimized for extracellular vesicle production. The RNA is expressed in the nucleus of the yeast cell, exported to the cytoplasm, translated into the biologically active protein, which is then taken into yeast extracellular vesicles through cytoplasmic sampling. Cells are removed from the growth media by centrifugation and filtering to yield a media fraction containing extracellular vesicles and free extracellular protein. The extracellular vesicles and protein are then precipitated from the media with polyethylene glycol (PEG, M.Wt. optimized for vesicle precipitation) and resuspended in a small volume of PBS. This mixture is then run over a size exclusion column (equilibrated in PBS) to isolate vesicles moving in the void volume from the free protein and PEG molecules, which are retained by the resin. The concentration of purified vesicles can then be determined from particle counting experiments, or absorbance readings at 260 nm for nucleic acid or 280 nm for protein. The purified vesicles are then added to mammalian target cells, which take up the vesicles and the biologically active protein. Release of the protein to the cytoplasm of the target cell then allows for biological activity.

FIG. 5. Schematic for delivery of a therapeutic RNA by targeted yeast vesicles. Yeast cells are transformed with a circular DNA plasmid encoding a fusion protein, consisting of an exosomal transmembrane protein and a targeting peptide, and the biologically active RNA of interest (yeast-specific promoters for each) and cultures are grown to a level of confluence optimized for extracellular vesicle production. The mRNA encoding the fusion protein is transcribed in the yeast nucleus, exported to the cytoplasm and translated and trafficked into the ER-Golgi to produce the membrane associated targeting fusion protein. The fusion protein is incorporated into the yeast extracellular vesicles, as these vesicles are derived from the plasma membrane. The RNA is expressed in the nucleus of the yeast cell, exported to the cytoplasm and taken into yeast extracellular vesicles through cytoplasmic sampling. Cells are removed from the growth media by centrifugation and filtering to yield a media fraction containing extracellular vesicles and free extracellular protein. The extracellular vesicles and protein are then precipitated from the media with polyethylene glycol (PEG, MWt optimized for vesicle precipitation) and resuspended in a small volume of PBS. This mixture is then run over a size exclusion column (equilibrated in PBS) to isolate vesicles moving in the void volume from the free protein and PEG molecules, which are retained by the resin. The concentration of purified vesicles can then be determined from particle counting experiments, or absorbance readings at 260 nm for nucleic acid or 280 nm for protein. The purified vesicles are then added to mammalian target cells carrying the receptors specific for the targeting ligand, which recognize that ligand and take up the vesicles and the biologically active RNA. Release of the RNA to the cytoplasm of the target cell then allows for biological activity.

FIG. 6. Membrane protein orientation for targeting ligands in different vesicle types. Fusion proteins that bring together transmembrane proteins with proteins to modify the surface of a vesicle (immune masking proteins or targeting ligands) must be properly oriented to the vesicle they modify. This schematic illustrates the different orientation that is required to modify post-Golgi vesicles (carrying fusion proteins in black) as compared to extracellular vesicles (carrying fusion proteins in grey). Post-Golgi vesicles bud from the Golgi apparatus and have outer surfaces common to the outer surface of the trans-Golgi network. Fusion proteins that modify this type of vesicle must therefore be oriented to face the cytosol, just as the outer surface of the Golgi network. Extracellular vesicles bud from the plasma membrane (in the case of ectosomes) or from the surface of multivesicular bodies (in the case of exosomes). In this case, fusion proteins that modify this type of vesicle must face away from the cytosol to have the proper orientation on the final extracellular vesicle.

FIG. 7. Autonomously replicating cytoplasmic linear DNA from Kluyveromyces lactis. The schematic shows the structure of a recombinant cytoplasmic linear DNA based on the pGKL1 and pGKL2 killer plasmids from K. lactis. The pGKL1 vector is dependent on the pGKL2 plasmid for replication and maintenance in the cell cytoplasm and carries open reading frames encoding for the heterotrimeric toxin (ORF2 and ORF4). The pGKL2 vector is the larger, autonomous vector that carries open reading frames for DNA and RNA binding proteins, DNA and RNA helicases, as well as DNA and RNA polymerases. Recombinant cytoplasmic linear DNA vectors are generated by subcloning of an insert containing a mammalian expression cassette into a unique BamHI site within ORF2 of the pGKL1 vector. This mammalian expression cassette disrupts the reading frame for the heterotrimeric toxin in ORF2 and consists of an optional DNA nuclear-targeting sequence (DTS), a mammalian/viral promoter, sequences encoding for the biologically active molecule and a mammalian polyA termination sequence. The insert may also include a second expression cassette consisting of an upstream conserved sequence (UCS) from the linear vector, a selectable marker appropriate for the vesicle-producing yeast strain and a yeast specific terminator sequence.

FIG. 8. Schematic for the formation of circular RNA. A DNA expression cassette for the production of circular RNA is illustrated. The 5′ end of the expression cassette includes a mammalian promoter sequence and the 3′ end carries a mammalian terminator sequence, which may include a polyA signal sequence for RNA polymerase II transcripts or a T-rich sequence for RNA polymerase III products. Between these sequences is an internal ribosome entry site (IRES), the biologically active RNA sequence, an optional polyA tract, and the circular RNA formation signals, consisting of inverted 5′ and 3′ splice sites for either the endogenous splicing machinery of the yeast cell or the self-splicing RNA cyclase ribozyme from the phage T4 group I intron. Transcription from the expression cassette produces a linear RNA product containing the 3′ splice site, the biologically active RNA sequence, the optional polyA tract and the 5′ splice site. This linear RNA intermediate is made circular through the activity of the endogenous splicing machinery in the nucleus of the target cell, or through the autocatalytic activity of the RNA cyclase ribozyme.

FIG. 9. Schematic for delivery of a circular RNA by yeast vesicles. Yeast cells are transformed with a DNA plasmid encoding the circular biologically active RNA of interest (yeast-specific promoters) and cultures are grown to a level of confluence optimized for extracellular vesicle production. The RNA is expressed and circularized in the nucleus of the yeast cell, exported to the cytoplasm and taken into yeast extracellular vesicles through cytoplasmic sampling. Cells are removed from the growth media by centrifugation and filtering to yield a media fraction containing extracellular vesicles and free extracellular protein. The extracellular vesicles and protein are then precipitated from the media and purified on a size exclusion column as described in FIG. 1. The concentration of purified vesicles can then be determined from particle counting experiments, or absorbance readings at 260 nm for nucleic acid or 280 nm for protein. The purified vesicles are then added to mammalian target cells, which take up the vesicles and the circular biologically active RNA. Release of the circular RNA to the cytoplasm of the target cell then allows for biological activity.

FIG. 10. Autonomously replicating cytoplasmic linear DNA producing a circular RNA. The schematic shows the structure of a recombinant cytoplasmic linear DNA based on the pGKL1 and pGKL2 killer plasmids from K. lactis. The pGKL1 and pGKL2 vectors are as described in FIG. 7 and FIG. 10. Recombinant cytoplasmic linear DNA vectors are generated by subcloning of an insert containing a mammalian expression cassette into a unique BamHI site within ORF2 of the pGKL1 vector. This mammalian expression cassette consists of an optional DNA nuclear-targeting sequence (DTS), a mammalian/viral promoter, sequences that direct formation of the circular RNA, sequences encoding for the biologically active molecule, an optional poly-adenosine tract and a mammalian termination sequence. The insert may also include a second expression cassette consisting of an upstream conserved sequence (UCS) from the linear vector, a selectable marker appropriate for the vesicle-producing yeast strain and a yeast specific terminator sequence.

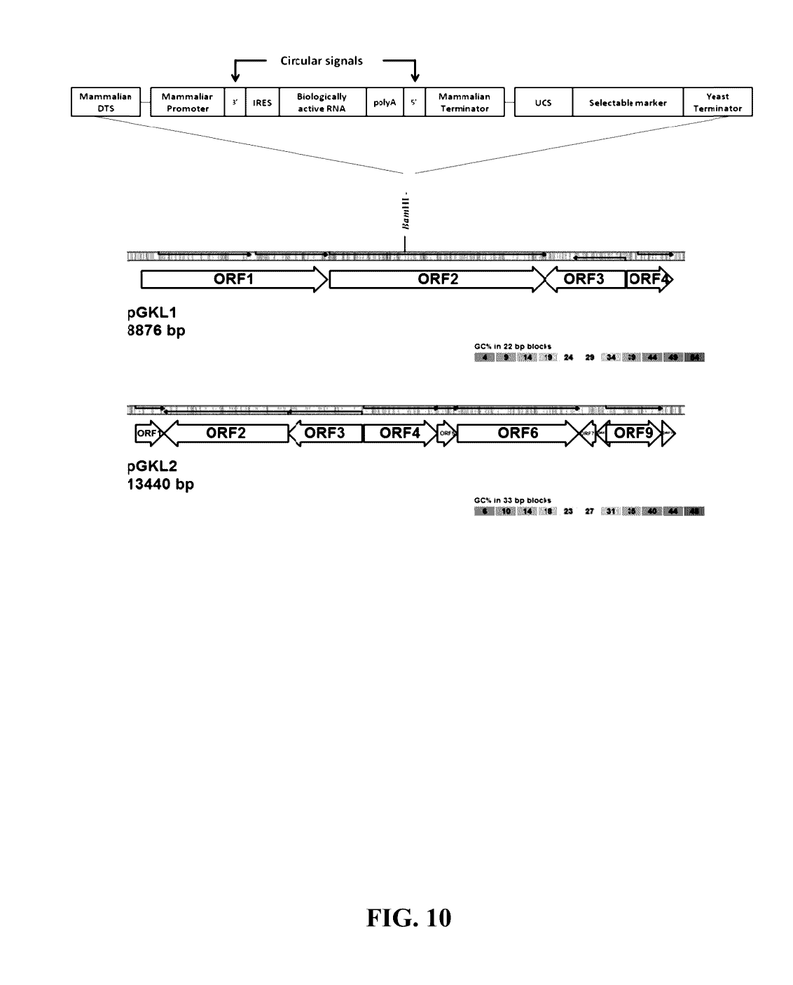

FIG. 11. Purification of yeast extracellular vesicles from the void volume of a size exclusion column. Candida glabrata cultures were grown at 37° C. for 48 hours (highly confluent) in YPD media. Cells were removed from the media by centrifugation (4000 rpm for 30 minutes) and the supernatant was filtered through 0.2-micron syringe filters. Extracellular vesicles were then precipitated from the media using 10% PEG (final concentration, Average M.Wt.=20,000 daltons, PEG20K) at 4° C. overnight. Precipitated extracellular vesicles were collected by centrifugation at 1500×g for 30 minutes and the pellet was resuspended in 0.5 mL of PBS. This vesicle fraction was then loaded to the top of a size exclusion column (Sepharose CL-6B) also equilibrated in PBS at 4° C. Fractions (1 mL) were collected and absorbance readings were taken at 260 nm (background subtractions with average reading from 350 to 450 nm) to construct the column profiles shown. Extracellular vesicles elute in fractions 7, 8 and 9, consistent with the expected void volume; free protein and PEG20K are retained by the resin and elute in a broad peak spanning fractions 16 through 24 (dark grey squares). The peak vesicle fraction, fraction 8, was then reloaded to the column and the process was repeated. The purified vesicles again run in the void volume fractions with no free protein present in later fractions (light grey squares).

FIG. 12. Particle size for purified yeast extracellular vesicles. The particle size of purified yeast vesicles was measured using a Brookhaven 90Plus Particle Size Analyzer. Purified particles were diluted into phosphate buffer saline and read under standard conditions.

FIG. 13. Extracellular vesicle production varies with the yeast strain. Extracellular vesicles were collected from the growth media for saturated cultures of S. cerevisiae, C. albicans and C. glabrala. All Cultures were Grown in 200 mL of YPD media for 72 hours at 30° C. for S. cerevisiae and C. albicans and 37° C. for C. glabrata. Vesicles were precipitated with PEG20K and analyzed on size exclusion columns equilibrated in phosphate buffered saline. Column profiles were constructed from absorbance readings at 280 nm, profiles include S. cerevisiae samples (black squares) and C. albicans (grey squares) and for C. glabrata (white squares).

FIG. 14. Increased yield of extracellular vesicles from Saccharomyces cerevisiae with a Chitin Synthase 3 deletion. Saccharomyces cerevisiae cultures (wild type and chs3Δ strains) were grown at 30° C. for 48 hours (highly confluent) in YPD media. Cells were removed from the media by centrifugation (4000 rpm for 30 minutes) and the supernatant was filtered through 0.2-micron syringe filters. Extracellular vesicles were then precipitated from the media using 10% PEG (final concentration, Average M.Wt.=20,000 daltons, PEG20K) at 4° C. overnight. Precipitated extracellular vesicles were collected by centrifugation at 1500×g for 30 minutes and the pellet was resuspended in 0.5 mL of PBS. This vesicle fraction was then loaded to the top of a size exclusion column (Sepharose CL-6B) also equilibrated in PBS at 4° C. Fractions (1 mL) were collected and absorbance readings were taken at 260 nm (background subtractions with average reading from 350 to 450 nm) to construct the column profiles shown. Extracellular vesicles and free protein isolated from the chs3Δ strain (dark grey squares) were more abundant than those isolated from the wild type strain (light grey squares).

FIG. 15. Increased yield of extracellular vesicles from Pichia pastoris upon addition of methanol. Pichia pastoris cultures were grown at 30° C. for 72 hours (highly confluent) in YPD media with or without methanol (0.5% final). Methanol was replenished every 24 hours to account for evaporation with the same 0.5% volume addition. Cells were removed from the media by centrifugation (4000 rpm for 30 minutes) and the supernatant was filtered through 0.2-micron syringe filters. Extracellular vesicles were then precipitated from the media using 10% PEG (final concentration, Average M.Wt.=5,000 daltons, PEG5K) at 4° C. overnight. Precipitated extracellular vesicles were collected by centrifugation at 1500×g for 45 minutes and the pellet was resuspended in 0.5 mL of PBS. This vesicle fraction was then loaded to the top of a size exclusion column (Sepharose CL-6B) also equilibrated in PBS at 4° C. Fractions (1 mL) were collected and absorbance readings were taken at 260 nm (background subtractions with average reading from 350 to 450 nm) to construct the column profiles shown. Extracellular vesicles and free protein isolated from the cells grown in methanol (dark blue squares) were more abundant than grown without methanol (light blue squares).

FIG. 16. Loading of an overexpressed RNA molecule to yeast extracellular vesicles. Yeast cells were transformed with a DNA plasmid encoding a reporter RNA molecule (GLP-1 transcript) and transformed cells were cultured with growth selection by auxotrophic markers. Cultures were grown at 30° C. for 48 hours (high saturation) and cells were removed by centrifugation. Cell pellets were resuspended in Qiazol buffer (Qiagen), disrupted with a bead beater (5 minutes of continuous homogenization) and RNAs were purified using miRNEasy kits according to the manufacturers protocol (Qiagen). Any remaining cells were then removed from the growth media by filtering through a 2-micron syringe filter. The extracellular vesicles were then precipitated from the growth media using 10% PEG (final concentration, Average M.Wt.=20,000 daltons, PEG20K) at 4° C. overnight. Precipitated extracellular vesicles were collected by centrifugation at 1500×g for 30 minutes, the pellet was resuspended in 0.7 mL of Qiazol (Qiagen) and RNAs were purified using miRNEasy kits according to the manufacturers protocol (Qiagen). Purified RNAs were used to generate cDNAs using a nested polyT primer in a reverse transcription reaction, which were then used as templates in qPCR experiments with nested primers and gene specific probes to selectively amplify the reporter RNA transcript. Amplification plots are shown for cell and extracellular vesicle fractions.

FIG. 17. Loading of an overexpressed polypeptide to yeast extracellular vesicles. Yeast cells were transformed with a DNA plasmid encoding a fusion protein consisting of the yeast exosomal protein Enolase, an RNA binding domain (Protein N from bacteriophage λ) and a streptavidin tag. Transformed cells were cultured with growth selection by auxotrophic markers for 48 hours (high saturation) and cells were removed by centrifugation. Cell pellets were resuspended in SDS loading buffer. Any remaining cells were then removed from the growth media by filtering through a 2-micron syringe filter. The extracellular vesicles were then precipitated from the growth media using 10% PEG (final concentration, Average M.Wt.=20,000 daltons, PEG20K) at 4° C. overnight. Precipitated extracellular vesicles were collected by centrifugation at 1500×g for 30 minutes, the pellet was resuspended in 0.5 mL of PBS and loaded to a size exclusion column (Sepharose CL-6B) equilibrated in PBS at 4° C. Fractions (1 mL) were collected and absorbance readings were taken at 260 nm (background subtractions with average reading from 350 to 450 nm) to construct the column profiles shown. Proteins were precipitated with TCA, washed with acetone and resuspended in SDS loading buffer. Cell lysates and column fractions were subjected to electrophoresis on a 4%-20% TRIS-glycine gradient gel and proteins were transferred to PVDF membranes for western blot analysis. Blots developed with primary antibodies specific to the streptavidin tag and HRP conjugated secondary antibodies, then visualized with DAB.

FIG. 18. Immune responses of mammalian cells upon treatment with purified yeast vesicles as determined by in vitro assay of TNF-α induction in RAW264.7 cells. Candida albicans cultures were grown at 30 C for 48 hours (highly confluent) in YPD media and extracellular vesicles were isolated as described before. Human macrophages (RAW264.7 cells) are cultured separately in DMEM media+10% fetal bovine serum (12-well plates, 100,000 cells per well) for 24 hours. Purified vesicles are then added to the macrophage cells (1 μg or 10 μg of total vesicular protein) in media containing either 10% fetal bovine serum or 10%/o of a vesicle-depleted serum. Untreated macrophages served as a negative control and macrophages treated with lipopolysaccharide (LPS) served as a positive control for immune activation. Growth media was collected from the macrophages 16 hours after addition of the vesicles and TNF-α concentrations were determined using an ELISA by comparison to a standard curve generated with a stock of recombinant protein. Results presented are average values calculated from triplicate samples+/−the standard deviations.

FIG. 19. Uptake of purified yeast extracellular vesicles into mammalian target cells. Candida glabrata cultures were grown at 37° C. for 48 hours (highly confluent) in YPD media and extracellular vesicles were isolated as described before. Purified vesicles were then labeled with a fluorescent lipid dye (PKH67, Sigma) at room temperature for 5 minutes. Labeled vesicles were purified away from free dye using a microcon filtering device with a 50-kDa M.Wt. cutoff, vesicles washed with PBS and then recovered in 200 μL of PBS. Labeled vesicles were then added to HUVEC, HEK293 and CT26 target cells and incubated at 37 C in a CO2 incubator for 24 hours. Uptake of fluorescently labeled vesicles was then visualized by microscopy using a fluorescent excitation lamp.

FIG. 20. A blot showing vesicles from the chs3Δ mutant of S. cerevisiae are enriched with ENO2 protein compared to vesicles from wild-type S. cerevisiae.

### DETAILED DESCRIPTION OF THE INVENTION

### Definitions

As used herein, the term “biologically active molecule” means any molecule produced in a cell that has a biological activity in vivo or in vitro. In some embodiments, the biologically active molecule is a DNA, a RNA, a protein, or a peptide.

As used herein, the term “biologically active RNA” is meant to refer to any RNA sequence that encodes for a peptide or protein or can bind to either a ligand, a peptide or a protein or can modulates gene expression or gene activity of targeted or non-specific gene products. In some embodiments, the biologically active RNA is not naturally occurring in yeast, e.g., the biologically active RNA is derived from mammalian RNA.

As used herein, the term “extracellular vesicle” is meant to refer to any lipid vesicle produced by yeast and secreted to the yeast extracellular space.

As used herein, the term “vesicle-producing cell” or “vesicle-producing yeast cell” is meant to refer to any yeast cell that produces a lipid vesicle that is secreted to the yeast extracellular space.

As used herein, the term “target cell” is meant to refer to any cell that takes up an extracellular vesicle or the contents of the extracellular vesicle. In some embodiments, the target cell is mammalian.

As used herein, the term “endogenously produced” is meant to refer to any molecule produced within a cell. In some embodiments, the cell is a vesicle-producing yeast cell.

As used herein, the term “heterogenous” means derived from or originating outside of the organism, e.g., the yeast.

As used herein, the term “non-pathogenic” is meant to refer to any yeast strain that is not known to cause disease in mammals.

As used herein, the term “cell wall biosynthesis enzyme” is meant to refer to any enzyme whose activity is related to production or maintenance of a cell wall in yeast.

As used herein, the term “transmembrane protein” is meant to refer to a protein expressed within a vesicle-producing yeast cell that spans the cell membrane and serves as an anchor for proteins that modify the surface of an extracellular vesicle.

As used herein, the term “targeting ligand” or “targeting peptide” is meant to refer to a protein that when expressed on the surface of an extracellular vesicle provides for targeted delivery of a vesicle and its contents to a specific cell type.

As used herein, the term “immune masking protein” or “immune masking peptide” is meant to refer to a protein that when expressed on the surface of an extracellular vesicle provides for reduced immunogenicity and toxicity in the biological system to which it is administered. In some embodiments, the biological system is a human.

As used herein, the term “fusion protein” is meant to refer to a protein comprising at least two polypeptides, which are operably linked. In some embodiments, the two polypeptides are derived from different sources (e.g., different proteins, species, or organisms). In some embodiments, the two or more polypeptides are operably linked, i.e., connected in a manner such that each polypeptide can serve its intended function. In some embodiments, the two polypeptides are covalently attached through peptide bonds.

As used herein, the term “expression vector” is meant to refer to any self-replicating polynucleotide sequence encoding for a biologically active molecule, e.g., RNA, DNA, protein, or peptide.

As used herein, the term “expression cassette” is meant to refer to a nucleic acid sequence capable of directing expression of a particular nucleotide sequence. In some embodiments, a plasmid or expression vector can comprise an expression cassette.

As used herein, the term “operatively linked” is meant to refer to an arrangement of flanking sequences wherein the flanking sequences so described are configured or assembled so as to perform their intended function.

### Yeast Extracellular Vesicles as Mammalian Delivery Systems

In certain aspects, the invention involves engineering yeast cells to produce and load biological macromolecules into yeast membrane vesicles. In further aspects, the loaded vesicles collect in the extracellular medium and can be purified for transfer into target cells or tissues. The present invention is different than what has been proposed for therapeutic applications involving other vesicles, e.g., PGVs.

First, the vesicles of the invention are secreted from the yeast cell and occupy the extracellular space as part of their cellular function. In contrast, previously disclosed PGVs are intracellular vesicles that function as intermediate carriers of secreted molecules. Such PGVs never exist in the extracellular space, but rather deliver their contents by fusing with the plasma membrane of a target cell.

Second, the extracellular vesicles of the invention are readily obtained from nonpathogenic yeast strains.

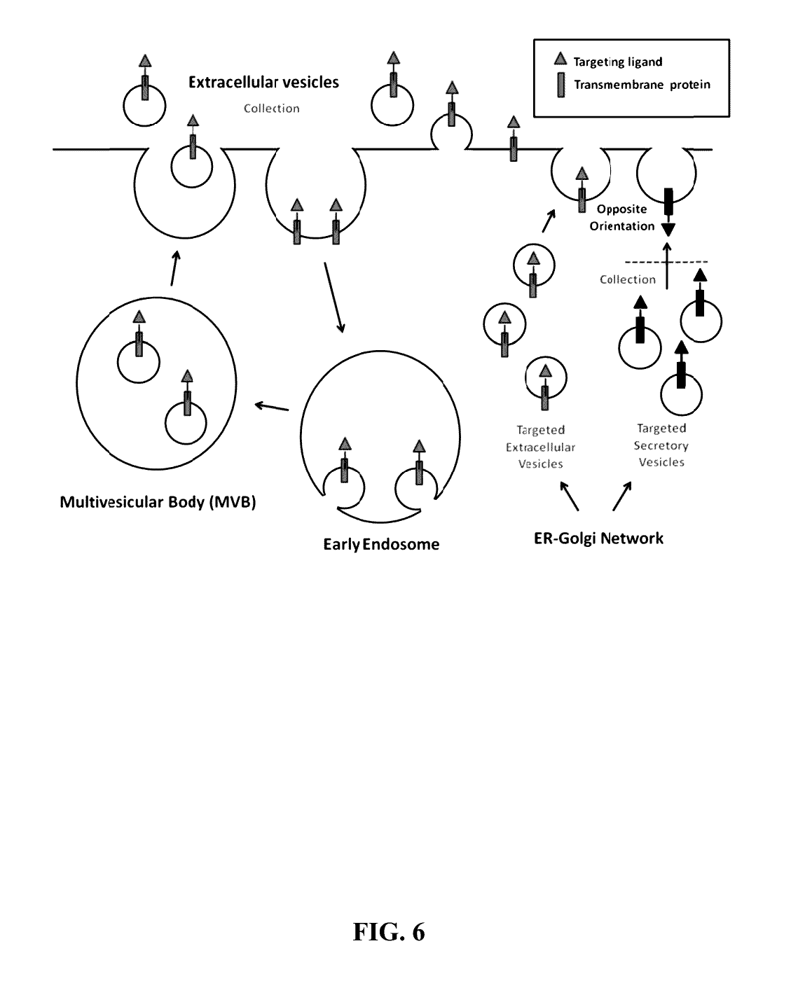

Third, the extracellular vesicles of the invention are derived from the plasma membrane in such a way that they share the orientation of the membranes and any associated proteins. In contrast, previously disclosed PGVs are collected from within the cell prior to fusion with the plasma membrane, meaning targeting ligands are oriented to the outside of these vesicles. In order to place targeting ligands on the outer surface of extracellular vesicles, these ligands must be located on the inner surface of the secretory vesicle (see FIG. 6). This is due to the fact that secretory vesicles deliver membrane proteins to the cell surface by fusing with the plasma membrane, such that the inner surface of the PGV becomes the outer surface of the plasma membrane.

Finally, the contents of the extracellular vesicles of the invention include not only cytoplasmic peptides and proteins, but also nucleic acids such as RNA and autonomously replicating cytoplasmic DNA. These nucleic acid molecules are excluded from the ER-Golgi network and could not be loaded into PGVs by any endogenous pathway currently known. The loading of peptides and proteins to PGVs is done through use of signal peptides that direct them to the ER-Golgi network. As this is the only known mechanism for loading PGVs, the potential cargo for this type of vesicle is limited to molecules comprising signal peptides.

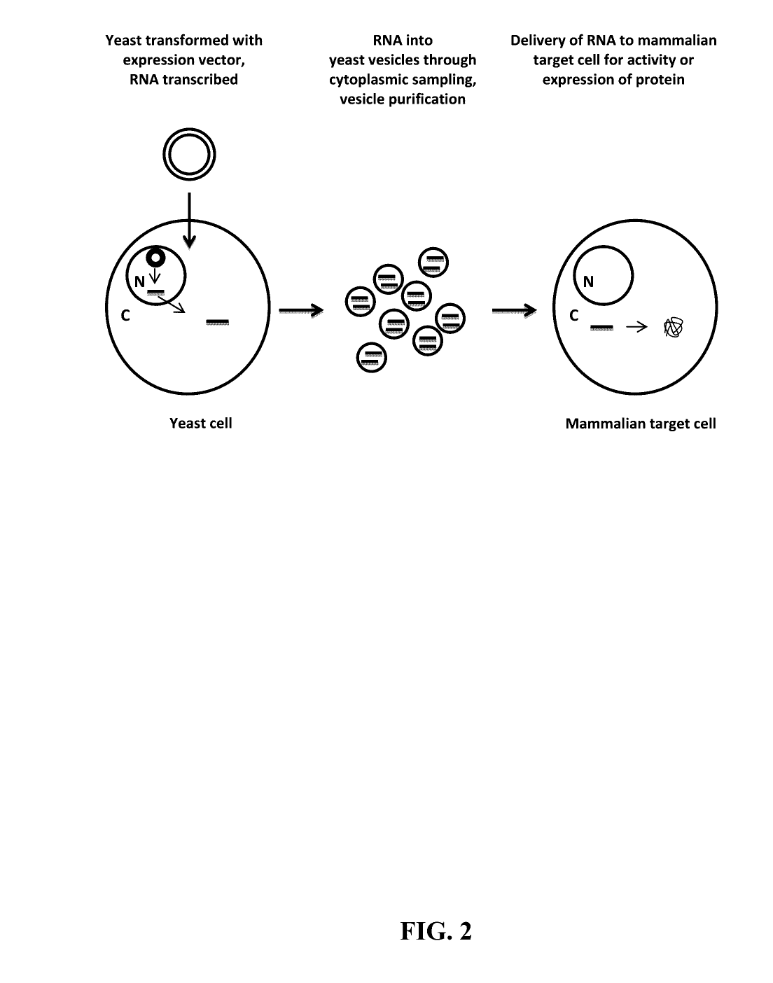

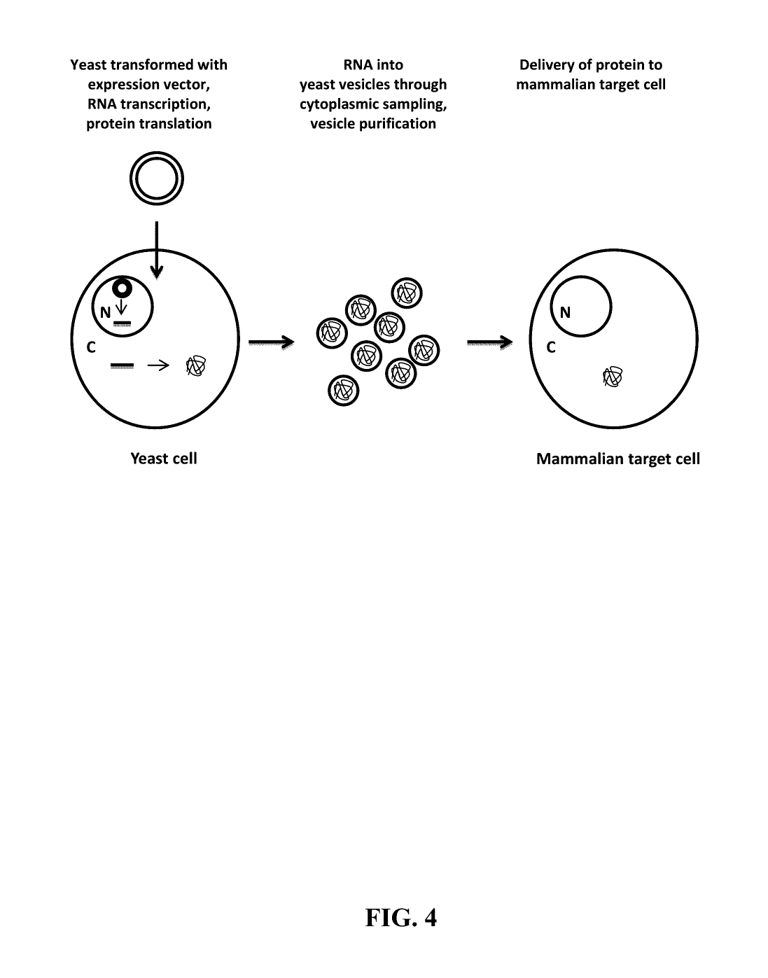

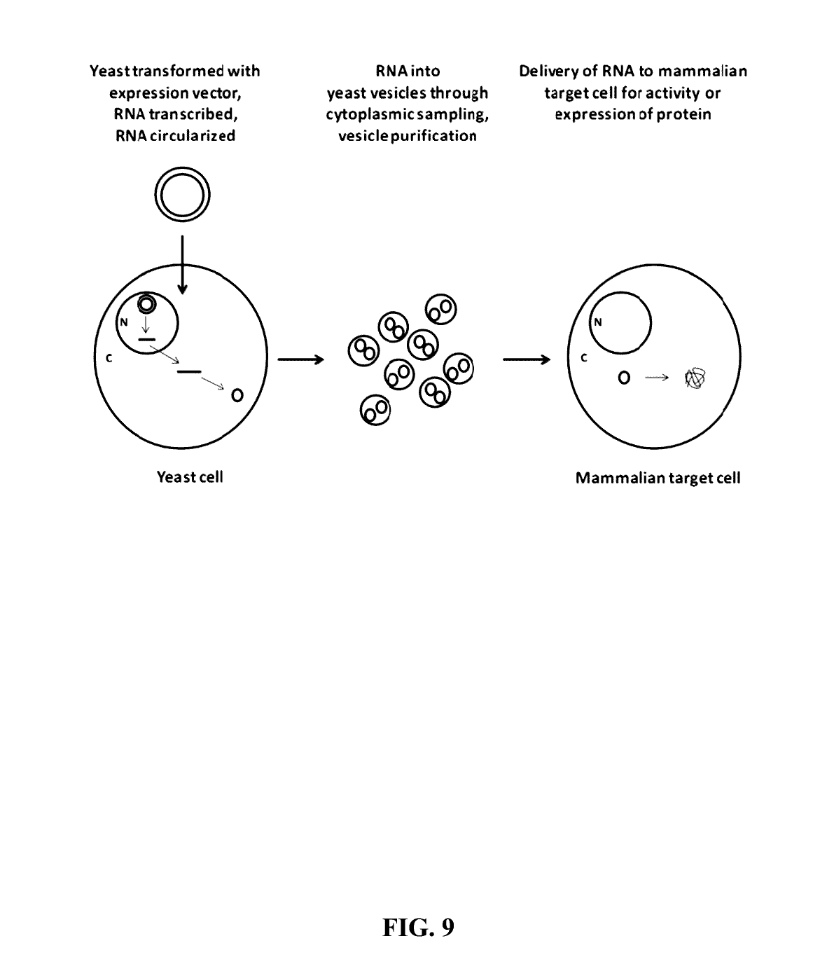

In certain aspects, the invention utilizes unmodified yeast extracellular vesicles as a delivery system for heterogenous biologically active molecules produced within the yeast cell (FIGS. 2-5, FIG. 9). In some embodiments, recombinant plasmids delivered, e.g., by transfection or transformation, to a vesicle-producing yeast cells encode biologically active molecules of the invention, e.g., DNA, RNA, peptides and/or proteins, which are produced using the biosynthetic pathways within the vesicle-producing yeast cells. These biologically active molecules accumulate in the cytoplasm of the yeast cells, where they are available for loading into extracellular vesicles. In some embodiments, upon delivery of an extracellular vesicle comprising a biologically active molecule to a target cell, the biologically active molecule acts to modify cellular functions through enzymatic, inhibitory or competitive functions directed to existing cellular proteins and/or nucleic acids in the target cell.

The claimed invention addresses at least two critical limitations of the therapeutic application of vesicles as delivery reagents for mammalian cells, namely the problems with loading of the biologically active cargo into the vesicles and the difficulty in obtaining vesicles at concentrations high enough for in vivo applications. Here, the biologically active molecules are produced within the yeast cell itself and loaded during formation of the vesicles, such that they emerge from the yeast cells ready to deliver their cargo to target cells. The high culture densities achievable with yeast and the relatively low cost of their growth media combine to yield vesicle production systems that are sufficiently concentrated for applications in vivo, scalable and cost effective.

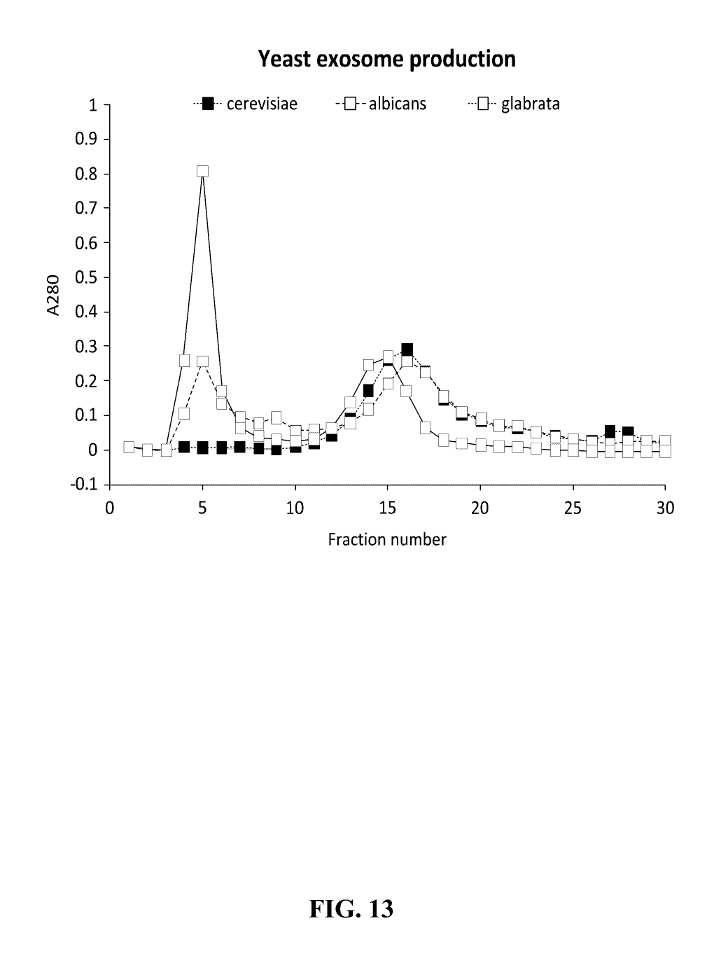

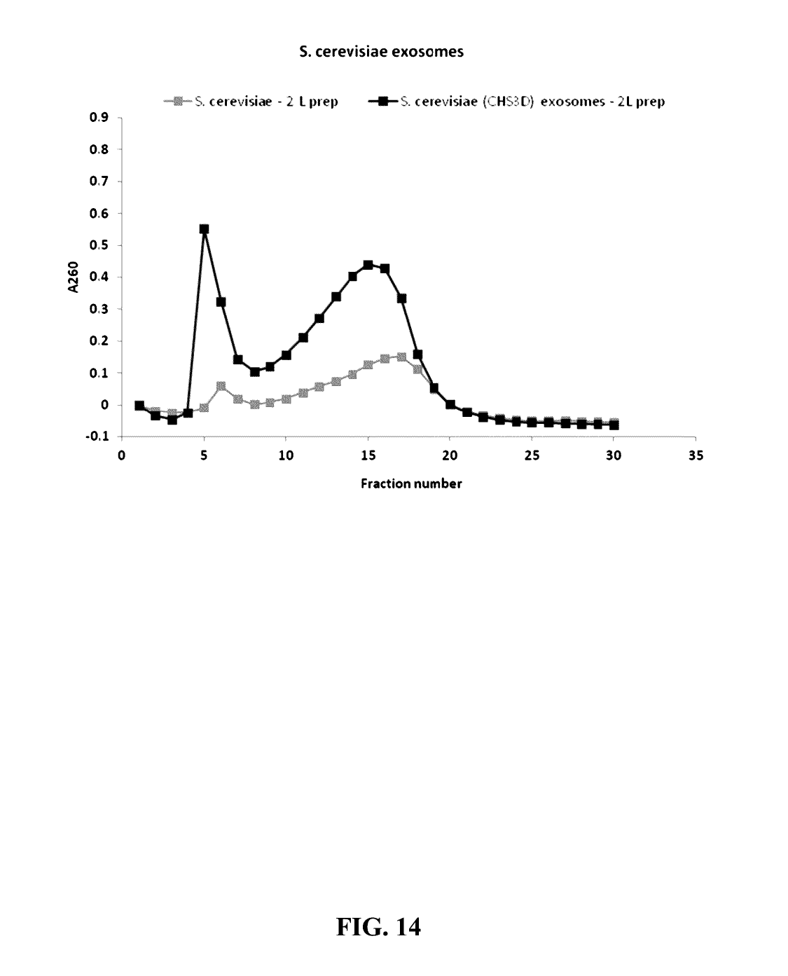

In certain embodiments, the efficiency of the secretion and vesicle loading process can be influenced by the conditions under which the vesicles are produced. Where the loading of biologically active molecules occurs through sampling of molecules present in the cytoplasm, systems that provide for greater cytoplasmic accumulation of those molecules can result in greater vesicle loading. As disclosed herein, the number of extracellular vesicles produced can vary with the strain of yeast (FIG. 13) and can be influenced by mutations that influence relevant pathways in the yeast cell, e.g., mutations in components of the cell wall biosynthesis pathway (FIG. 14). In some embodiments, optimized vesicle loading and/or production can be obtained through screens of various yeast strains (and mutants within those strains) to determine conditions where both the biologically active molecule and vesicle accumulation levels are the highest.

### Vesicle-Producing Yeast Cells

In certain embodiments, the vesicle-producing yeast cells of the invention can include non-pathogenic yeast strains. In some embodiments, the yeast strain is Saccharomyces cerevisiae, Saccharomyces kluyveri, Candida albicans, Candida glabrata, Kluyveromyces lactis, Pichia pastoris, Pichia etchellsii, Pichia acacia, Debaryomyces hansenii, Wingea robertsiae, Neurospora crassa, Schizosaccharomyces pombe, Aspergillus nidulans, Penicillium camemberti, Penicillium chrysogenum, Monascus purpureus, Saccharomyces boulardi, Zygosaccharomyces rouxii, or mutant strains thereof.

In certain embodiments, the yeast cells that produce extracellular vesicles are identified by analyzing conditioned growth media for the presence of secreted vesicles. Vesicle output can be compared across strains (wild type and mutant) by culturing equal numbers of cells, purifying vesicles from the growth media and quantifying the number of vesicles through direct measurement, e.g., in a Nanosight instrument, or through application of standard curves relating vesicle counts to either protein content (A280 measurements) or nucleic acid content (A260 measurements). In certain embodiments, the vesicle-producing yeast cells of this invention are generated upon transformation with an expression vector that produces the biologically active molecules. In some embodiments, transformants are obtained by growth on antibiotic selective media, where the expression vectors carry genes that confer resistance to the antibiotic. This selective pressure can be maintained on the growing cultures throughout the vesicle production process, ensuring that only vesicles derived from cells carrying the expression vector are generated.

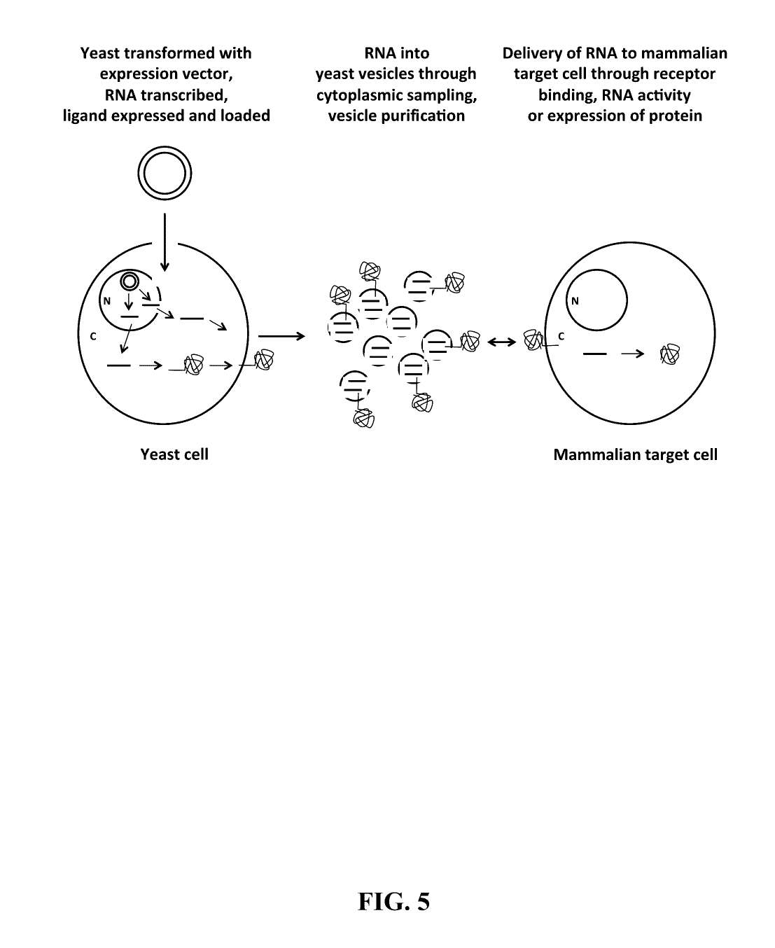

The vesicle-producing yeast cells of the invention can also be transformed with expression vectors that encode for fusion proteins. In some embodiments, the fusion proteins comprise of transmembrane proteins linked to immune masking proteins and/or targeting ligands (FIG. 5). These fusion proteins are expressed in the yeast cells and incorporated into the plasma membrane. Since the extracellular vesicles have membranes derived from the plasma membrane, these fusion proteins will also reside on the vesicles (FIG. 6). In some embodiments, the immune masking fusion proteins provide a reduced immune response [65-68] when the vesicles are administered to a target organism and/or the targeting ligands provide for delivery to specific target cell types [69-77].

The fusion proteins of the invention can be produced by standard recombinant DNA techniques. For example, a DNA molecule encoding the first polypeptide is ligated to another DNA molecule encoding the second polypeptide, and the resultant hybrid DNA molecule is expressed in a host cell to produce the fusion protein. In some embodiments, the DNA molecules are ligated to each other in a 5′ to 3′ orientation such that, after ligation, the translational frame of the encoded polypeptides is not altered (i.e., the DNA molecules are ligated to each other in-frame).

### Yeast Extracellular Vesicles

Another aspect of the invention is directed to the extracellular vesicles of the invention and their use as drug delivery reagents. In particular, the current invention provides a solution to the problem of loading biologically active molecules to vesicles and obtaining vesicles in great enough abundance to be useful for commercial and in vivo applications. Use of the described yeast cell produced vesicles provides an alternative to synthetic delivery systems and offers the potential for low toxicity, low immune responses, efficient drug delivery to target cells and straightforward modification of vesicles through yeast genetics. The isolated yeast vesicles can be used as a transfection reagent for mammalian cells growing in cell culture or administered to animal model systems, e.g., by local or systemic injection. In some embodiments, appropriate culturing and vesicle purification is used in order to obtain a concentrated stock of reagent vesicles.

Extracellular vesicles are produced by yeast cells growing in culture, being transported across the cell membrane and cell wall, and being deposited in the extracellular space. Once in the extracellular space, the vesicles can be collected from the growth media, purified and concentrated using a number of different methods known in the art. In some embodiments, the vesicle-producing cells, extracellular proteins and extracellular debris are separated and removed from the vesicles. Additional methods can be employed to further separate subpopulations within the extracellular vesicles, e.g., based on differences in vesicle size, surface properties, or biogenesis pathways.

In some embodiments, vesicles of different size can be isolated using sucrose or glycerol gradients by ultracentrifugation.

Vesicles can carry on their endogenous surface membrane proteins, which can vary depending on the biogenesis pathway, or the presence of recombinant proteins added to specifically alter some property of the vesicle. In some embodiments, surface proteins can be used to purify these vesicles using affinity chromatography with antibodies specific to a surface protein.

In one embodiment, yeast cells are removed from the culture media by centrifugation and filtering; and the yeast vesicles are precipitated with polyethylene glycol (PEG) at 4° C. overnight. Precipitated vesicles can be collected by centrifugation and re-suspended in a minimal volume of phosphate buffered saline. Vesicles can be separated from extracellular proteins and precipitating PEG with size exclusion chromatography, where the vesicles run in the void volume and elute in early fractions and the contaminants are retained by the resin and elute in later fractions. The vesicle fractions can be collected and pooled for use, either at the concentrations at which they are eluted from the column or after further concentration with Centricon Filtering Devices.

Certain aspects of the invention are directed to a yeast-derived extracellular vesicle loaded with an endogenously produced biologically active molecule. In some embodiments, this extracellular vesicle can be generated by transforming yeast cells with an expression vector for the biologically active molecule, culturing those cells under conditions where that biologically active molecule is loaded into the extracellular vesicles, collecting the vesicles from the growth media, and purifying the vesicles comprising the biologically active molecule. Subsequently, the purified vesicles comprising the biologically active molecule can be administered to a target cells in vitro or in vivo.

Extracellular vesicles can be formed by a number of different pathways in a vesicle-producing cell [60, 61]. The most direct route for vesicle formation is by membrane blebbing (ectosomes), where vesicles are formed directly at the plasma membrane [62-64]. Exosomes are derived from a number of intermediate, intracellular organelles (endosomes and multivesicular bodies), which are necessary for the formation and release of the vesicles to the extracellular space. In both of these vesicle forming pathways, the interior of the vesicle is derived from the cytoplasm of the cell. Therefore, loading of the biologically active molecules to the interior of the vesicles can occur through accumulation of that biologically active molecules in the cytoplasm of the vesicle producing cell, where loading can be concentration-dependent and/or driven by mass action.

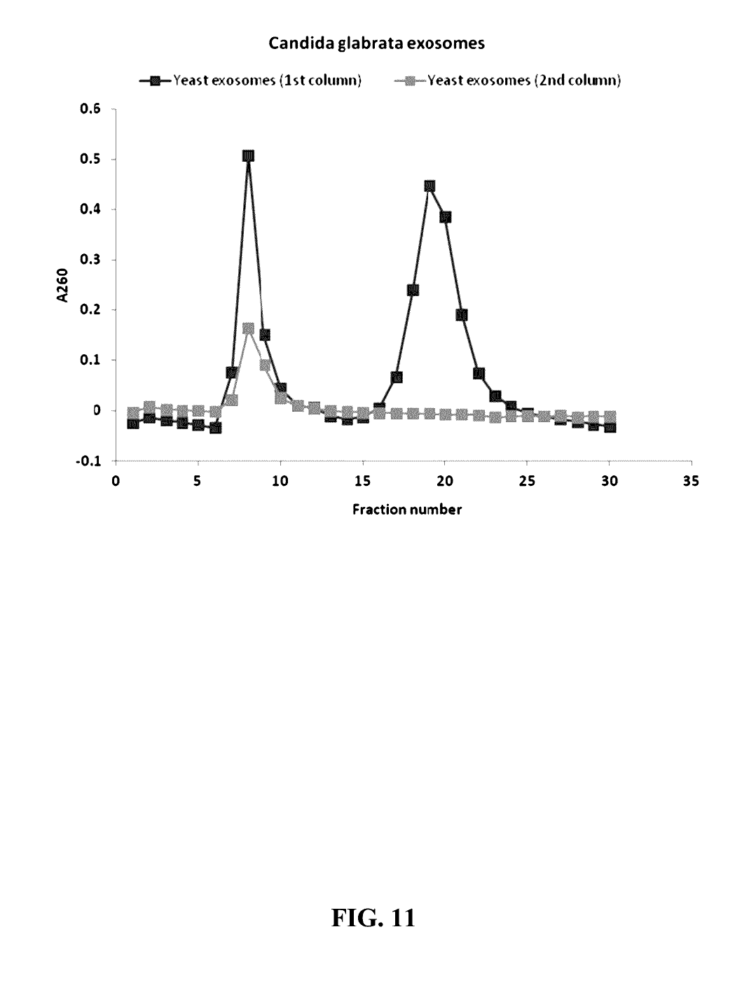

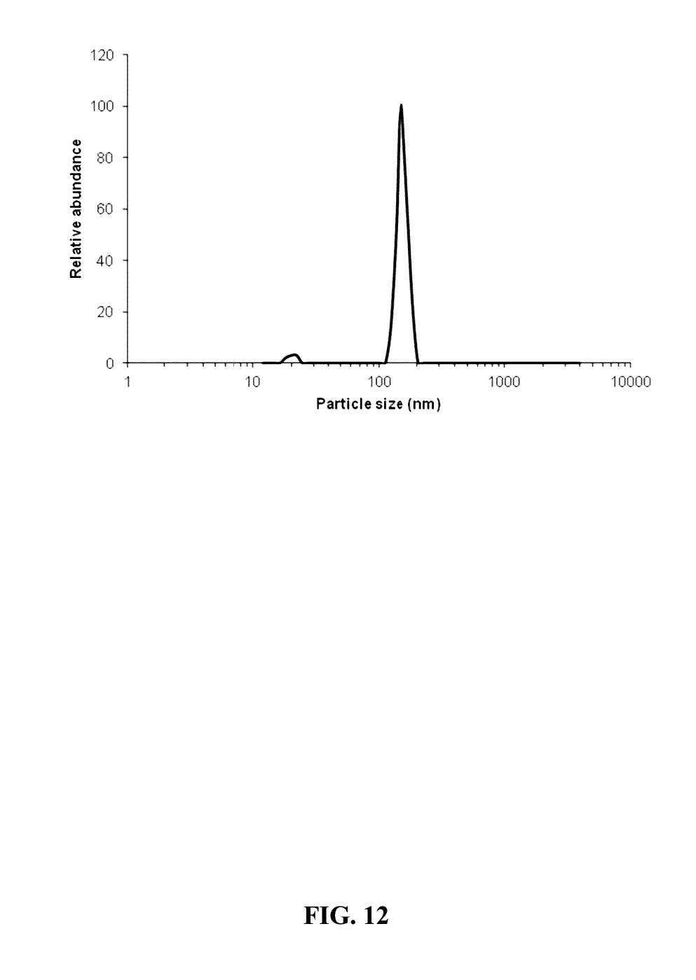

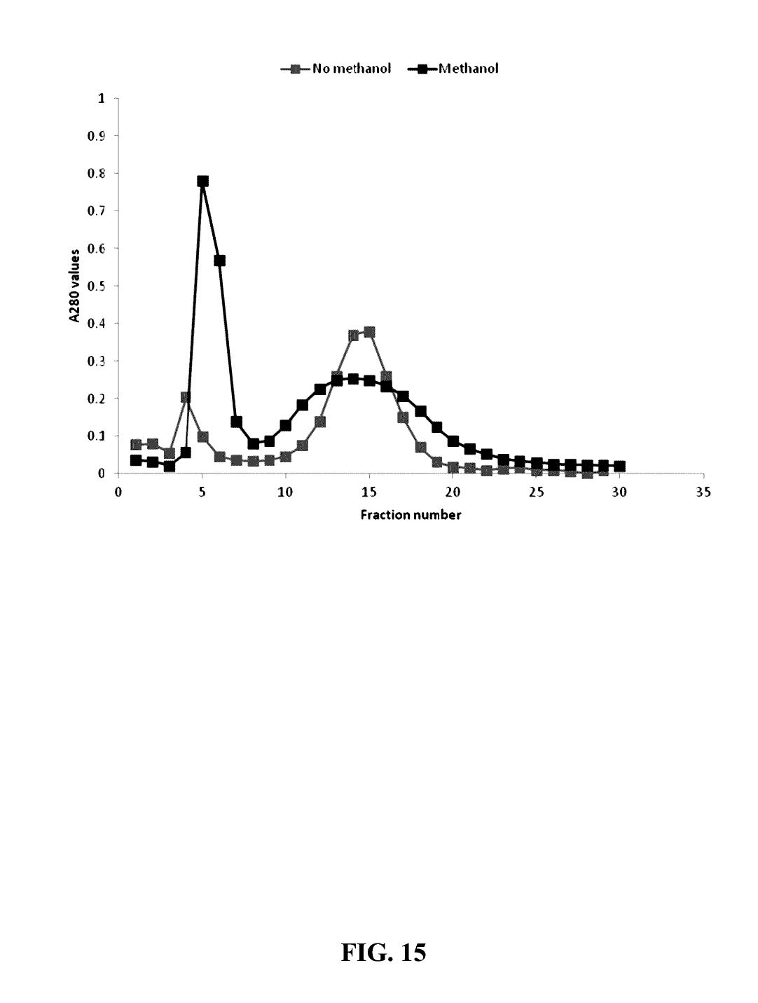

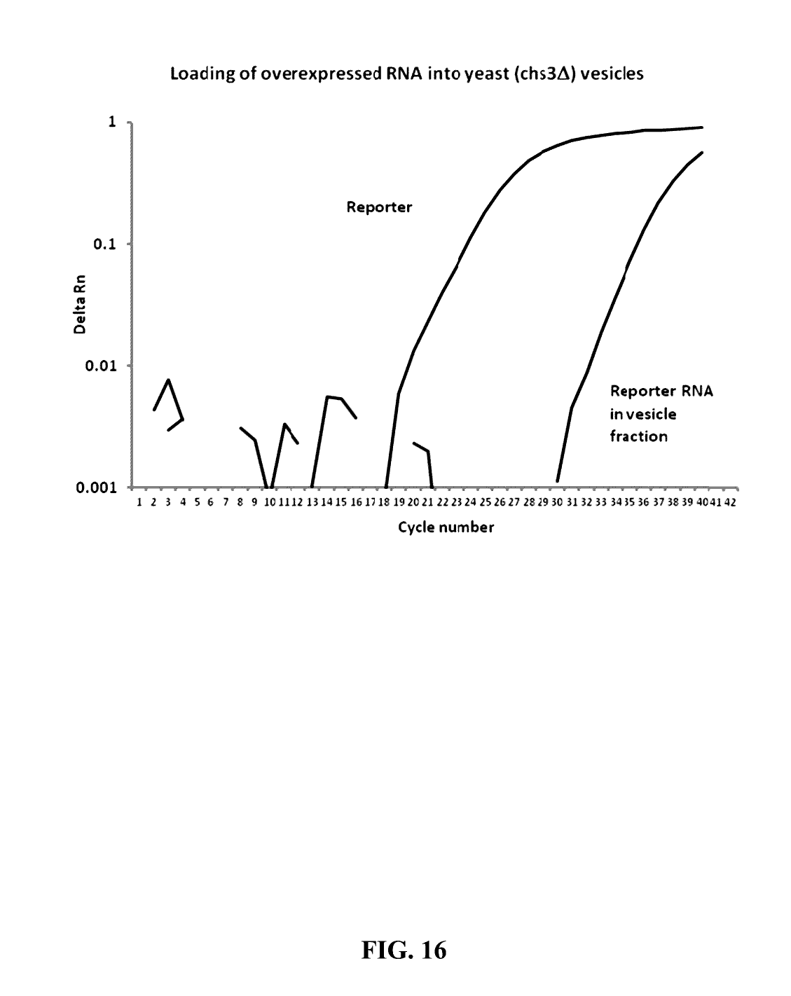

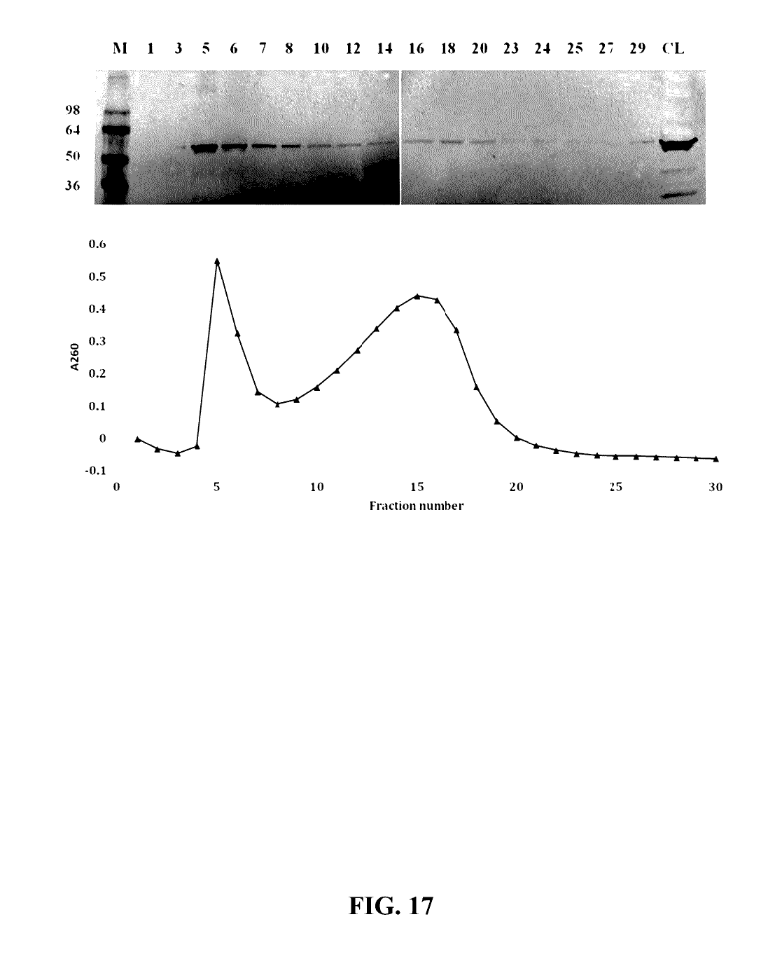

Yeast extracellular vesicles are secreted from the yeast cells and accumulate in the growth media of yeast cultures over time. As disclosed herein, the vesicles can be purified from the void volume of a size exclusion column (FIG. 11). In some embodiments, the vesicles have particle diameters in the hundreds of nanometers (FIG. 12). The number of vesicles produced can depend on the yeast strain used (FIG. 13) and can be modulated by mutations to a yeast strain, e.g., the deletion of the chitin synthase 3 gene from S. cerevisiae (FIG. 14) or the addition of methanol to P. pastoris cultures (FIG. 15). In some embodiments, the vesicles can be loaded with an overexpressed RNA molecule (FIG. 16) or an epitope tagged, expressed protein (FIG. 17).

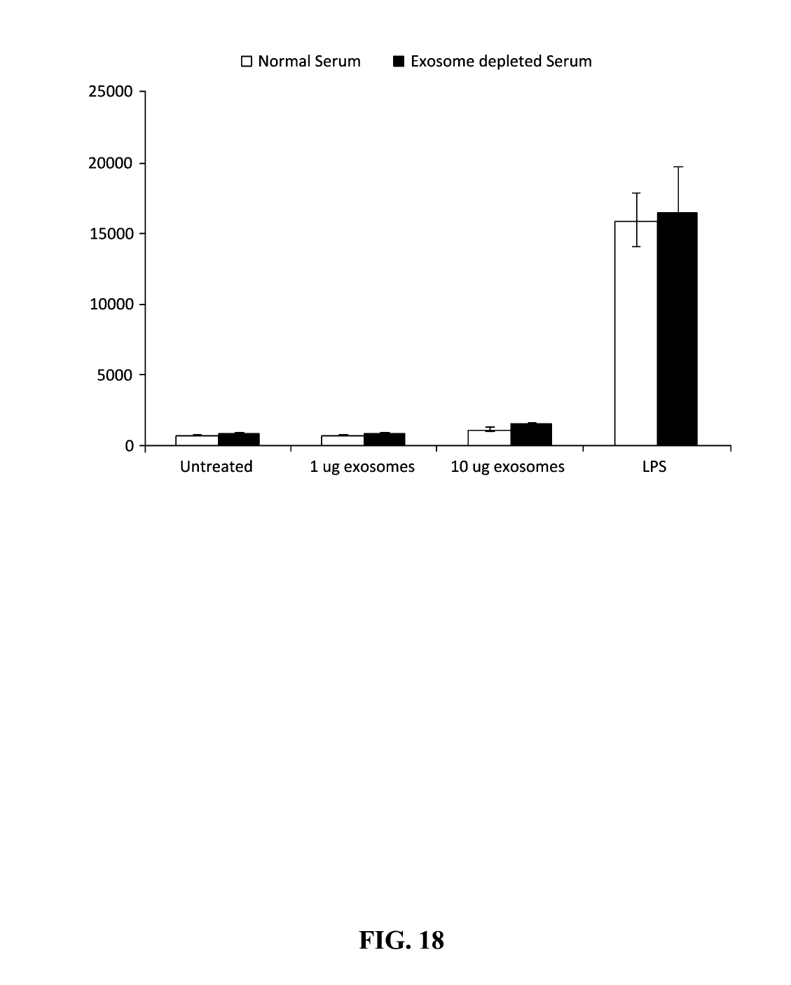

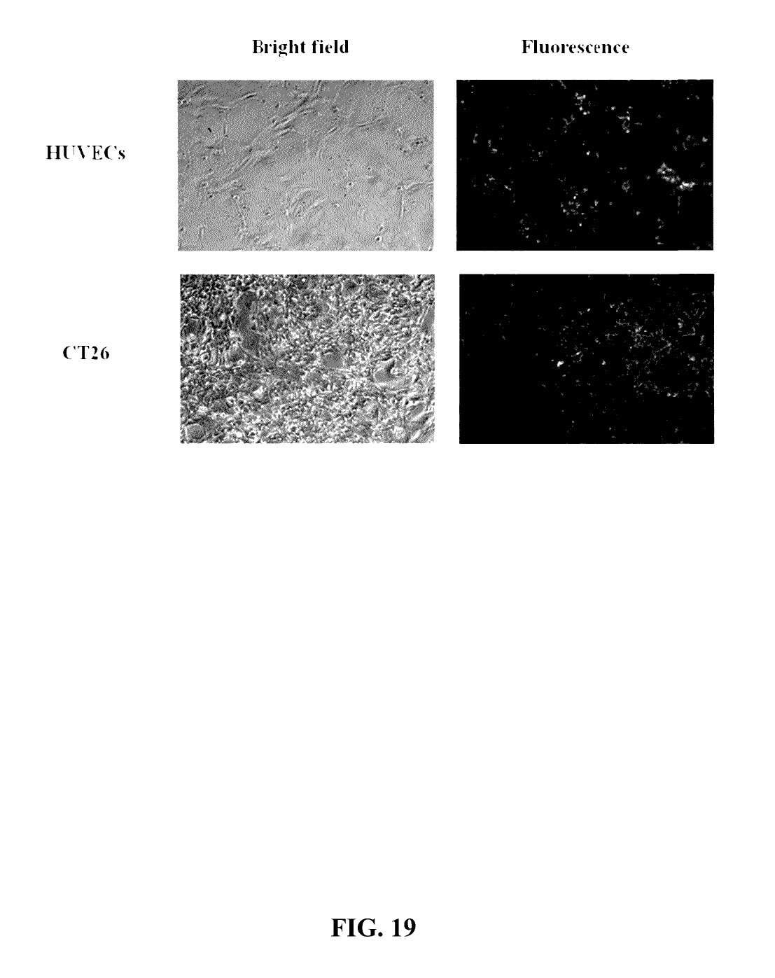

In some embodiments, the vesicles show minimal activation of TNF-α in cultured human macrophages (FIG. 18) and are readily taken up by mammalian target cells in vitro (FIG. 19). In some embodiments, the vesicles can also be modified to carry proteins on their outer surface that provide immune masking functions and/or cell targeting functions.

Extracellular vesicles have been reported for a variety of yeast cells, normally playing a role in virulence by interacting with host cells [19-21]. As discussed herein, extracellular vesicles produced by yeast offer a possible solution to the problem of production scale. Yeast cultures can achieve significantly higher cell densities and large scale culturing is considerably more cost effective. Though these vesicles have the potential to trigger immune responses, especially when derived from pathogenic strains, the immunogenicity of vesicles from the commensal/non-pathogenic strains of the invention are well suited for use in mammalian systems.

### Membrane Proteins and Immune Masking Peptides

The genetic pliability of yeast allows for vesicle modifications that can alleviate issues of toxicity and immunogenicity, e.g., through the introduction of membrane proteins with immune masking function. In addition, expression of membrane fusion proteins carrying established targeting ligands can also allow for targeted delivery to cell types expressing complementary receptors.

In some embodiments, yeast cells of the invention producing the extracellular vesicles are transfected or transformed with a DNA plasmid comprising a polynucleotide that expresses (i) a biologically active RNA molecule and (ii) an mRNA transcript encoding for a fusion protein comprising a transmembrane protein and/or an immune masking peptide (FIG. 5). The vesicle-producing yeast cell is generated by administering to the cell one or more expression vectors designed to produce at least one biologically active RNA molecule through electroporation, enzymatic digestion or alkali cation transformation. The expressed RNA molecules are delivered to the cytoplasm of the yeast cell through endogenous nuclear export machinery, where the mRNA transcript is translated into protein, which is incorporated into the cell membrane. As the extracellular vesicles are also derived from the cell membrane, vesicles from these yeast cells will also carry the fusion protein in their membranes. The biologically active RNA molecules are incorporated into yeast extracellular vesicles through random sampling of the cytoplasm. These vesicles accumulate in the growth media, allowing for separation from the yeast cells, purification and use as a delivery reagent. Upon systemic administration to animal systems, the immune masking peptides provide for a reduced immune response and delivery of the biologically active RNA to the mammalian target cell allows for function inside the target cell.

### Biologically Active RNA Molecules

Loading of endogenously produced biologically active molecules into yeast vesicles requires that those molecules be present in the cytoplasm of the vesicle-producing yeast cell. Biologically active RNA, protein, and peptides can readily accumulate in the cytoplasm.

In certain embodiments, the plasmids or vectors of this invention have the capacity to encode a RNA molecule which is loaded into an extracellular yeast vesicle for subsequent delivery to and function within a mammalian target cell. These RNA molecules can exert a biological effect through a number of different mechanisms depending on the cellular components with which they interact. In some embodiments, the biologically active RNAs function through base pairing interactions with specific mRNA transcripts that lead to translational silencing or degradation of the mRNA molecule. Two related classes of inhibitory RNAs are antisense RNA molecules and small inhibitory RNA molecules. The antisense RNA is typically a direct complement of the mRNA transcript it targets and functions by presenting an obstacle to the translational machinery and also by targeting the transcript for degradation by cellular nucleases. The small inhibitory RNA (siRNA) molecules act through the post-transcriptional gene silencing (PTGS) pathway or through the RNA interference (RNAi) pathway. These RNAs are approximately 22 nucleotides in length and associate with specific cellular proteins to form RNA-induced silencing complexes (RISCs). These small RNAs are also complementary to sequences within their mRNA targets and binding of these complexes leads to translational silencing or degradation of the transcripts.

Another advantage of the invention applies when the biologically active RNA is an shRNA or miRNA molecule. Many yeast strains lack most or all of the components of the RNAi pathway [22, 23]. This provides a mechanism by which shRNA and/or miRNA molecules can be delivered intact to yeast vesicles. Similar production approaches in mammalian cells can be influenced by competition with the endogenous RNAi pathway, resulting in less shRNA available for loading into vesicles.

Two additional classes of RNA molecules that can modulate gene expression are the catalytic RNA ribozymes and the RNA aptamers. Ribozymes are RNA based enzymes that catalyze chemical reactions on RNA substrates, most often hydrolysis of the phosphodiester backbone. Formation of the catalytic active site requires base pairing between the ribozyme and the RNA substrate, so ribozyme activity can also be targeted to desired substrates by providing appropriate guide sequences. When targeted to mRNA transcripts, ribozymes have the potential to degrade those transcripts and lead to down-regulation of the associated protein. RNA aptamers are typically selected from pools of random RNA sequences by their ability to interact with a target molecule, often a protein molecule. Engineering RNA aptamers is less straightforward as the binding is not defined by base pairing interactions, but once an effective sequence is found the specificity and affinity of the binding often rivals that of antibody-antigen interactions. RNA aptamers also have a greater range of target molecules and the potential to alter gene activity via a number of different mechanisms. This includes direct inhibition of the biological activity of the target molecule with no requirement for degradation of the protein or the mRNA transcript which produces it.

In some embodiments, the plasmids or expression vectors of the invention can encode for mRNA transcripts which, in turn, encode for biologically active peptides and proteins. The mRNA are transcribed in the vesicle-producing yeast cell, but the biologically active peptide or protein can be produced through translation of the mRNA in either the yeast cell or upon delivery of the mRNA transcript to a mammalian target cell. In some embodiments, the encoded peptides and proteins modulate cellular activity through enzymatic activity, interactions with cellular proteins or interactions with cellular nucleic acids. These functions can occur within the target cell itself, or, in the case of transcripts encoding proteins carrying signal sequences, act in the extracellular space upon secretion from the target cell via the ER-Golgi pathway.

### Circular RNA Molecules

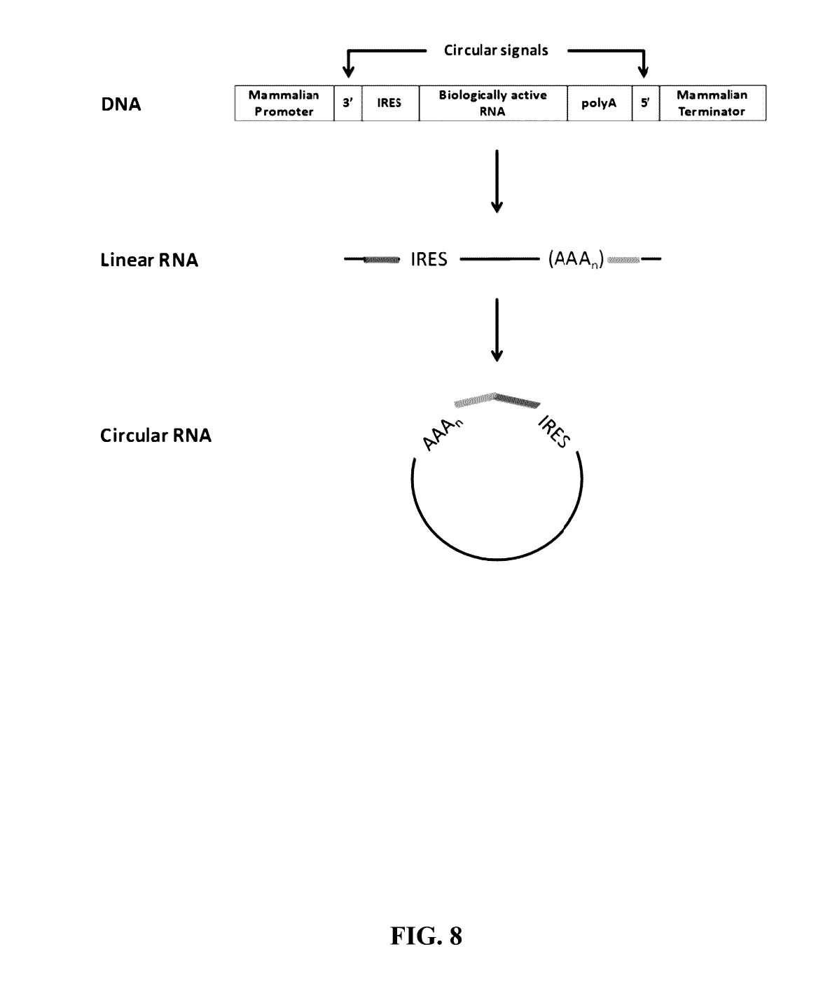

In certain aspects of the invention, the biologically active RNA sequences described is loaded into yeast vesicles in either a linear or circular form. Circular forms of the RNA can have a stability advantage upon delivery to the target cells, as they will not be substrates for RNA exonucleases [78, 79]. This stability can be of particular importance for RNA molecules that would normally undergo significant turnover, such as mRNA transcripts. These circular RNA molecules can be formed through different synthesis pathways, the activities of which are directed by sequences flanking the RNA of interest. Circular RNAs can be produced through the normal splicing pathway, where the 5′ and 3′ splice sites are transposed in a process known as back-splicing (FIG. 8) [80]. The RNA circularization process can occur in the yeast cell prior to loading into the yeast vesicles or in the mammalian target cell upon delivery. Alternatively, circular RNAs can be produced by ribozyme sequences derived from the Group I intron of the td gene from phage T4 [81]. In this example, the 3′ and 5′ splice sites are also rearranged so that the product of the splicing reaction is circular. However, this process occurs spontaneously within the RNA sequence and does not require any additional splicing factors from the vesicle-producing yeast cell or the target cell.

The circular form of an mRNA transcript requires specialized elements in order to be translated in the target cell. As the circular RNA lacks the 5′ cap structure typical of mRNA transcripts, an alternative mechanism of translation will be needed to produce the biologically active protein. In some embodiments, a first element is an internal ribosome entry site (IRES), a sequence element from viruses (e.g., the picornaviruses like Encephalomyocarditis virus, EMCV, and Poliovirus, PV) that normally allows for cap-independent initiation of translation of the viral genome [82-85]. This element appears to remove the need for translational initiation factors through direct interactions with the ribosomal 40S subunit, though the efficiency of this process is considerably lower than what is observed for cap-dependent initiation. In some embodiments, a second element is a poly-adenosine tract to mimic the polyA tail of a functional mRNA [86]. This element has been reported to improve the translation from circular RNA transcripts. Another type of naturally occurring circular RNA, known as miRNA sponges, can also be generated in yeast and loaded to yeast vesicles [87-92]. These RNAs are believed to play a role in regulating miRNA mediated post-translational gene silencing and are comprised of repetitive RNA elements that are complementary to miRNAs. Thus, these RNAs have the potential to act as alternative binding sites, potentially sequestering miRNAs and miRNA complexes and modulating their interaction with target sites located in mRNA transcripts.

### Autonomously-Replicating Cytoplasmic Linear DNA

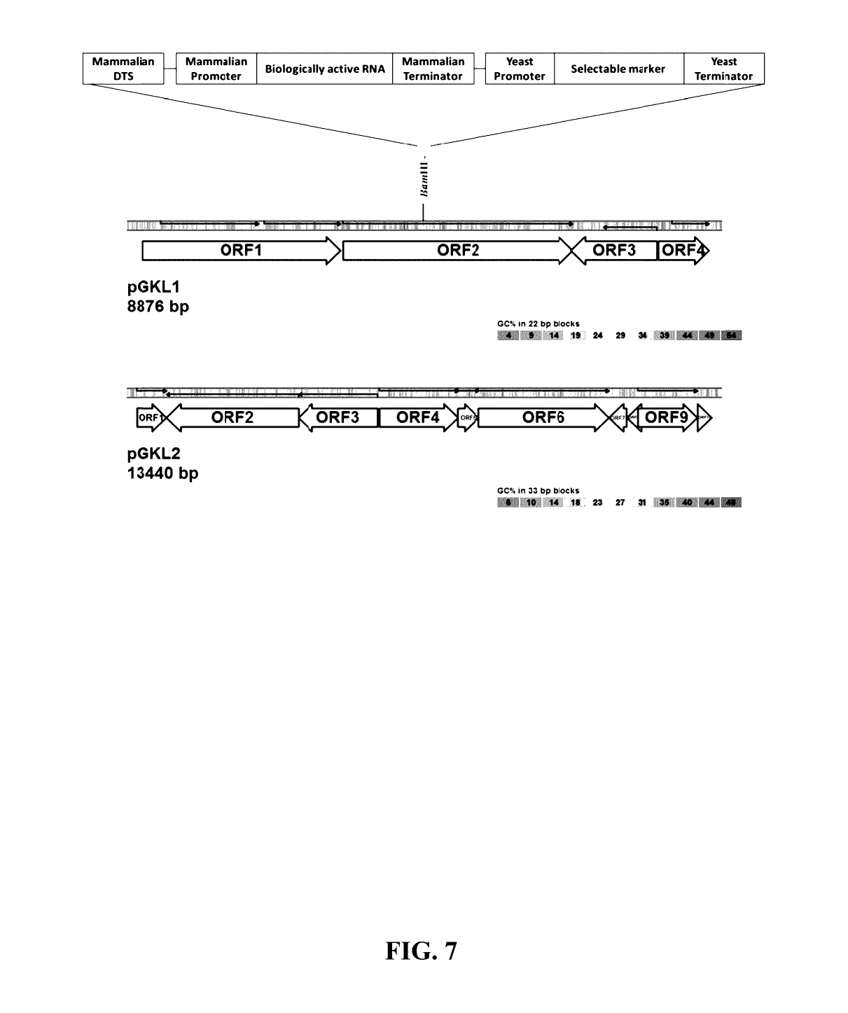

Autonomously replicating DNA vectors found in the cytoplasm of yeast cells [35, 47] can be loaded into yeast extracellular vesicles for delivery to and expression in mammalian target cells. These DNA vectors are linear in nature with lengths on the order of 10³-10⁴ base pairs and contain a number of densely spaced open reading frames under the control of unique cis-acting elements termed upstream conserved sequences (UCSs, see FIG. 7). These DNA elements are maintained independent of the nuclear replication machinery and therefore carry all the elements necessary for replication in these open reading frames. The linear vectors can exist as single, independent elements or in groups of two or three vectors, where one vector is autonomous and the others are dependent. These linear vectors have been used to create recombinant vectors that express foreign genes in yeast carrying the vectors [93-95]. The autonomously replicating DNA vectors of this invention are unique in that they combine elements from yeast and mammalian systems for expression of the biologically active molecule within the mammalian target cell. Specifically, the open reading frames responsible for maintenance of the linear DNA vector in the yeast cytoplasm (a location that allows for loading into the yeast vesicles) are combined with mammalian expression cassettes, which allow for production of the biologically active molecule upon delivery to the mammalian target cells. The mammalian expression cassettes can include a UCS from one of the open reading frames to allow for gene expression in the cytoplasm of the target cell, or it can carry a mammalian/viral promoter alongside an optional DNA nuclear-targeting sequence (DTS) for localization and expression in the target cell nucleus.

Therefore, the cytoplasmic DNA vectors can comprise, among other things, sequences encoding for proteins necessary for the maintenance of the cytoplasmic DNA including, but not limited to, DNA or RNA polymerases, DNA or RNA helicases and DNA or RNA binding proteins, as well as an optional DNA nuclear-targeting sequence (DTS) and a promoter/terminator sequence for expression of the RNA in the mammalian target cell. In one embodiment, the cytoplasmic DNA element comprises an expression cassette that encodes a biologically active RNA sequence. Expression cassettes for the RNA components of the cytoplasmic DNA are prepared by PCR amplification of the relevant sequences from RNA expressing plasmids using the appropriate forward and reverse primers. Primers include sequences complementary to the biologically active RNA sequence, sites for restriction enzymes used in subcloning and about six GC base pairs at the 5′ end of each primer to facilitate digestion with restriction enzymes. This expression construct is digested with appropriate restriction enzymes for subcloning into the backbone construct, which places the RNA expression cassette downstream from a yeast-specific promoter sequence and upstream of a yeast-specific termination sequence. Alternatively, expression vectors can be constructed by recombination cloning with primers containing sequences flanking the restriction sites that are complementary to the cloning site in the backbone vector.

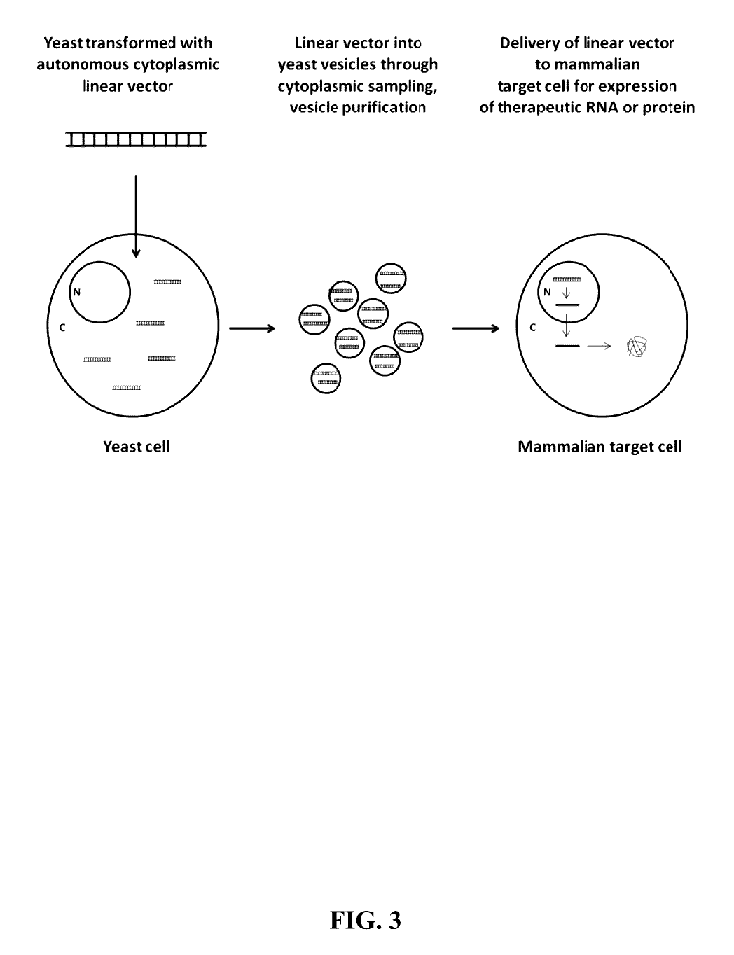

Cytoplasmic accumulation of DNA is not as common. Autonomously replicating linear DNA molecules have been identified in the cytoplasm of a number of yeast strains [24-44]. These extra-nuclear DNA elements are associated with a killer phenotype that is directed against a variety of potential competitor yeast, providing a mechanism for dominating a particular growth environment. This killer phenotype is conveyed by two genes carried by the extra-nuclear DNA, one encoding for a secreted exotoxin and the other encoding for a protein that conveys resistance to that toxin. These DNA elements occur as single vectors, pairs, or triplets of vectors that, in addition to the killer genes, carry genes necessary for maintenance of the vectors in the cytoplasm [45-50]. In certain embodiments, these autonomously replicating cytoplasmic elements serve as backbone vectors for the engineering of recombinant vectors carrying therapeutic genes (in place of the killer genes) available for loading to vesicles from the cytoplasm of the yeast cell (FIG. 3). Upon delivery of the vesicles to a mammalian target cell, these recombinant vectors would express the therapeutic genes of interest from expression cassettes carrying either promoter sequences derived from other linear DNA open reading frames (for expression in the cytoplasm) or viral promoters with DNA nuclear-targeting sequences (for expression in the nucleus).

In some embodiments, the yeast cells producing the extracellular vesicles are transformed with an autonomously replicating cytoplasmic linear DNA encoding a biologically active RNA molecule such as a ribozyme, an antisense nucleic acid, an aptamer, a short interfering RNA (siRNA), a double-stranded RNA (dsRNA), a micro-RNA (miRNA), and a short hairpin RNA (shRNA) molecule, as well as RNA transcripts encoding one or more biologically active peptides or proteins. These RNAs can have either a linear or circular form. In some embodiments, the circular RNAs can also include miRNA sponges. The cytoplasmic linear DNA is generated using an appropriate backbone plasmid (or plasmids) capable of autonomous replication in the yeast strain of interest. The expression cassette for the biologically active RNA molecule is cloned into the backbone plasmid using standard molecular biology techniques and plasmid stocks are prepared from large-scale cultures of transformed yeast cells. Administering to the cell through electroporation, enzymatic digestion or alkali cation transformation one or more expression vectors designed to produce at least one biologically active RNA molecule generates the vesicle-producing yeast cell. The cytoplasmic linear DNA molecules are maintained in the cytoplasm of the yeast cell, where they are incorporated into yeast extracellular vesicles through random sampling of the cytoplasm. These vesicles accumulate in the growth media, allowing for separation from the yeast cells, purification and use as a delivery reagent. Upon delivery to a mammalian target cell, the biologically active RNA is expressed from the cytoplasmic linear DNA that carries a mammalian specific promoter sequence.

### Expression Vectors

Expression vectors (including expression cassettes) containing polynucleotides encoding biologically active molecules (e.g., RNA, proteins or peptides) are also encompassed by the invention.

Expression vectors of the invention can be composed of DNA or RNA, and may be linear or a closed circular plasmid. The vector system may be a single vector or plasmid or two or more vectors that together contain or control the replication, integration and/or expression of the polynucleotides of the invention in a yeast cell. Polynucleotides of the invention can be inserted into the vector in either a forward or reverse orientation with respect to any particular promoter sequence contained in the vector.

The RNA expression vectors of the invention can comprise, among other things, a pUC origin of replication and a drug resistance gene, such as a kanamycin resistance gene, allowing for preparation of the plasmid in bacteria, as well as an origin of replication for propagation in yeast, and a promoter/terminator for expression of the RNA in the yeast cell. In one embodiment, the expression vector comprises an expression cassette that encodes a biologically active RNA sequence.

Expression cassettes for the RNA components of the yeast expression plasmid are prepared by PCR amplification of the relevant sequences from RNA expressing plasmids using the appropriate forward and reverse primers. Primers include sequences complementary to the biologically active RNA sequence, sites for restriction enzymes used in subcloning and about six GC base pairs at the 5′ end of each primer to facilitate digestion with restriction enzymes. This expression construct is digested with appropriate restriction enzymes for subcloning into the backbone construct, which places the RNA expression cassette downstream from a yeast-specific promoter sequence and upstream of a yeast-specific termination sequence. Alternatively, expression vectors can be constructed by recombination cloning with primers containing sequences flanking the restriction sites that are complementary to the cloning site in the backbone vector.

Expression cassettes for the protein components of the yeast expression plasmid are prepared by PCR amplification of the relevant sequences from cDNA clones using the appropriate forward and reverse primers. Primers include sequences complementary to the domain of interest, sites for restriction enzymes used in the subcloning, and six GC base pairs at the 5′ end of each primer to facilitate digestion with restriction enzymes. Initiation codons and optimized Kozak translational start sites are added to each primer corresponding to the 5′ end of the transcript to promote translation of the N-terminal domains of each fusion protein. Restriction sites are added to the primer corresponding to the 3′ end of the transcript to facilitate assembly of delivery domains with RNA binding domains. Domains are linked to one another directly or via sequences encoding alpha helical linker domains. These linkers provide separation between the two functional domains to avoid possible steric issues.

The present invention provides expression vectors useful in the production of the nucleic acid molecules for loading into yeast extracellular vesicles. In one embodiment, the invention provides an expression vector that expresses one or more biologically active RNA sequences of the invention (FIG. 2). The expression vector additionally comprises a first promoter sequence, a termination sequence, and optionally one or more primers sequences, wherein the polynucleotide encoding the biologically active RNA sequence is operably linked to the promoter sequence and the termination sequence. The RNA encoded on the expression vector is expressed in the nucleus of the transfected yeast cell and are transported to the cytoplasm through endogenous nuclear export machinery, where they are incorporated into yeast extracellular vesicles through random sampling of the cytoplasm. Alternatively, these RNA molecules could encode for biologically active protein molecules, in which case these mRNA transcripts are translated into protein, which can also be incorporated into yeast extracellular vesicles through random sampling of the cytoplasm. The biologically active RNA sequences can be one or more different types of biologically active RNA sequences directed to the same gene target or can be biologically active RNA sequences directed to different gene targets. The biologically active protein can be one or more different type of protein sequences directed to a particular cytoplasmic function or the disruption of a cytoplasmic process involving one or more different gene targets.

In some embodiments, the expression vector or expression cassette comprises a promoter operably linked to a nucleotide sequence of interest that may be operably linked to termination signals. It also can include sequences required for translation of the nucleotide sequence. In some embodiments, the coding region codes for a peptide of interest or a biologically active RNA of interest. The expression vector or expression cassette comprising the nucleotide sequence of interest may be chimeric. In some embodiments, the expression cassette is in a recombinant form useful for heterologous expression. In some embodiments, an expression vector or expression cassette comprises a nucleic acid sequence comprising a promoter sequence, a polynucleotide encoding a peptide sequence or a polynucleotide encoding an RNA sequence, and a terminator sequence.

In some embodiments, a flanking sequence operably linked to a coding sequence can influence the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered operably linked to the coding sequence.

### Intrabodies

Intrabodies are antibody proteins (or fragments of antibody proteins) that have been adapted to function in the cytoplasm of a target cell and recognize intracellular protein targets [58, 96-98]. As antibodies are secreted proteins that reside and function in the extracellular space, their synthesis and folding occurs in the endoplasmic reticulum, where chaperones and enzymes allow formation of the properly folded, functional protein structures. These structures typically include di-sulfide bonds which link the protein subunits together, further stabilizing the active antibody structure. Protein folding chaperones are typically restricted to the endoplasmic reticulum and di-sulfide bonds are not favored in the reducing environment of the cytoplasm, circumstances that lead to reduced antibody activity when antibodies are translated and folded in the cytoplasm. Intrabodies are stabilized versions of existing antibodies or novel antibodies and antibody fragments generated specifically for cytoplasmic function. These intrabodies bind to target molecules (usually proteins) and act as inhibitors for the target protein's normal cellular function. In this invention, intrabodies are made in the vesicle-producing yeast cell and loaded to vesicles for delivery to and function in the mammalian target cell.

Antibodies that are expressed in the cytosol of cells and bind to intracellular target proteins are known as intrabodies [58, 59]. Intrabodies can be expressed in the target cell where they function or delivered to that target cell with an appropriate delivery system. Antibodies are secreted proteins that fold in the endoplasmic reticulum, where proper conformations are achieved through the activities of chaperones and the formation of disulfide bonds. Intrabodies are designed for folding and stability in the cytoplasm, where the chaperone activity is not present and the more reducing environment does not favor disulfide bonds. Once delivered to the cytoplasm, these stabilized intrabodies can interact with their target proteins with a high degree of specificity, disrupting intracellular processes through competitive binding of the target proteins. The function of intrabodies inside the cell provides access to a large set of target molecules with potential therapeutic value.

In some embodiments, the yeast cells producing the extracellular vesicles of the invention are transfected or transformed with a DNA plasmid comprising a polynucleotide that expresses an mRNA transcript encoding for an intrabody. The vesicle-producing yeast cell is generated by administering to the cell one or more expression vectors designed to produce at least one intrabody targeting one or more genes of interest through electroporation, enzymatic digestion or alkali cation transformation. The expressed mRNA transcript is delivered to the cytoplasm of the yeast cell through endogenous nuclear export machinery, where the mRNA transcript is translated into the intrabody protein. In some embodiments, the intrabody protein molecules are incorporated into yeast extracellular vesicles through random sampling of the cytoplasm. These vesicles accumulate in the growth media, allowing for separation from the yeast cells, purification and use as a delivery reagent. In some embodiments, upon delivery to the mammalian target cell, the intrabody is available for function inside the target cell.

### CRISPR Complexes

The prokaryotic immune mechanism made up of clustered regularly interspaced short palindromic repeats (CRISPR) provides bacteria and archaea with protection from foreign nucleic acids contained in viruses and plasmids [99-103]. At the heart of this system is the CRISPR DNA locus consisting of CRISPR-associated genes (Cas genes), a leader sequence and a repeat spacer array. The CRISPR spacers are derived from foreign genetic elements and confer a form of acquired immunity by serving as templates for CRISPR RNA (crRNA), which forms complexes with endonucleases from the Cas genes to guide the cleavage activity to the foreign DNA. In one example of CRISPR activity, the Cas9 endonuclease pairs with an RNA duplex consisting of a crRNA and a trans-activating crRNA (tracrRNA) to form a guide RNA (gRNA) sequence that directs Cas9 mediated DNA cleavage. Cas9-crRNA complexes are recruited to potential target sites by protospacer adjacent motifs (PAMs), where the DNA duplex is unwound and an RNA-DNA duplex is formed. These interactions trigger Cas9 DNA endonuclease activity, leading to cleavage of the DNA strands. This activity is analogous to the RNA guided cleavage of RNA by siRNA/RISC complexes and the DNA cleavage activity of the gRNA/Cas9 complexes can be utilized for genome editing using the canonical rules of Watson-Crick base pairing to create site specific guide sequences. In this invention, gRNA/Cas9 complexes are made in the vesicle-producing yeast cell and loaded to vesicles for delivery to and function in the mammalian target cell.

Genome editing processes utilizing the zinc-finger proteins and subsequent applications with the TALENs (synthetic nucleases) have been greatly simplified by application of the CRISPR (clustered regularly interspaced short palindromic repeats) editing system taken from bacteria and archaea [51-57]. Whereas these early systems required custom proteins for each individual genomic target sequence, the activity of the Cas9 DNA endonuclease is directed by the CRISPR RNA (crRNA) guide sequence, the design of which requires only the canonical rules of Watson-Crick base pairing. Cas9-crRNA complexes are recruited to potential target sites by protospacer adjacent motifs (PAMs), where the DNA duplex is unwound and an RNA-DNA duplex is formed. These interactions trigger Cas9 DNA endonuclease activity, leading to cleavage of the DNA strands. Thus, CRISPR/Cas systems can be directed to produce double stranded breaks at defined positions in a genome of interest through engineering of appropriate crRNA guide strands, a process that alters or disrupts the activity of the targeted genes.

In some embodiments, the yeast cells producing the extracellular vesicles of the invention are transformed with a DNA plasmid comprising a polynucleotide that expresses an mRNA transcript encoding for the CRISPR Cas9 protein and the CRISPR RNA (crRNA) guide sequence specific for the target site. The vesicle-producing yeast cell is generated by administering to the cell one or more expression vectors designed to produce at least one crRNA guide sequence targeting one or more genes of interest through electroporation, enzymatic digestion or alkali cation transformation. The expressed RNA molecules are delivered to the cytoplasm of the yeast cell through endogenous nuclear export machinery, where the mRNA transcript is translated into the Cas9 protein, which then binds to the crRNA guide sequence. In some embodiments, the Cas9/crRNA complexes are incorporated into yeast extracellular vesicles through random sampling of the cytoplasm. These vesicles accumulate in the growth media, allowing for separation from the yeast cells, purification and use as a delivery reagent. In some embodiments, upon delivery to the mammalian target cell, the Cas9/crRNA complexes are available for function inside the target cell.

### EXAMPLES

### Example 1

### Preparation of Yeast Vesicles Loaded with Endogenously Produced RNA for Delivery to Mammalian Cells

Expression vectors for the endogenously produced RNA are constructed from isolated plasmid backbones and PCR amplified expression cassettes for the biologically active RNA. The expression vector should include at least the following components: an origin of replication for preparation in bacteria, an antibiotic selectable marker for selection in bacteria, an origin of replication for propagation in yeast, a promoter and terminator for expression of the RNA, both of which are appropriate for the yeast strain being used. Non-limiting examples of suitable backbone vectors include those derived from pRS413, pRS414, pRS415, pRS416, pRS423, pRS424, pRS425, pRS426, etc. These plasmid backbones contain a pMB1/ColE1 origin of replication (from pBR322) and an ampicillin resistance gene allowing the vector to be replicated in bacteria and cultured in the presence of ampicillin. The backbones also include the 2 micron replication origin for replication in yeast, as well as yeast specific promoters (including GPD, TEF, Gal1, PDC1, PDC2 and AOX1) and terminators (including CYC1, TEF1 and cgHIS3) for expression of the RNA.

Expression cassettes for the biologically active RNA or the mRNA encoding the biologically active polypeptide are prepared by annealing DNA oligos, in the case of small RNAs, or by PCR amplification of the relevant sequences from cDNA clones, in the case of mRNA transcripts, using the appropriate forward and reverse primers. Primers typically include sequences complementary to the sequence(s) of interest, sites for restriction enzymes used in the subcloning, and at least four GC base pairs at the 5′ end of each primer to facilitate digestion with restriction enzymes. Other useful primers can include sequences complementary to the domain(s) of interest, sites for restriction enzymes used in the subcloning, and 15 bases of vector sequence flanking the restriction site for use in recombination cloning (In-fusion Advantage PCR cloning kit, Clontech, Catalog #639620). A typical PCR reaction contains 10 mM Tris-HCl pH 9.0, 50 mM KCl, 1.5 mM MgCl₂, 0.1% Triton X-100, 200 μM each dNTP, 1.0 μM sense primer, 1.0 μM antisense primer, 100 ng DNA template and 1.0 U of Taq polymerase per 50 μL reaction. Reactions are cycled through 3 temperature steps: a denaturing step at 95° C. for 30 seconds, an annealing step at 50° C. to 60° C. for 30 seconds and an elongation step at 72° C. for 1 minute. Typically, the total number of cycles ranges from 20 to 35 cycles depending on the specific amplification reaction. Ligation reactions are set up PCR expression cassettes and plasmid backbones digested with restriction enzymes and purified on 2% agarose gels run in 1×TAE and excised bands are recovered using Qiagen's Qiaex II gel purification system. A typical ligation reaction contains 30 mM Tris (pH 7.8), 10 mM MgCl₂, 10 mM DTT, 1 mM ATP, 100 ng DNA vector, 100 to 500 ng DNA insert, 1 unit T4 DNA ligase and is ligated overnight at 16° C. Alternatively, expression vectors can be constructed by recombination cloning. A typical recombination reaction contains 1× In-fusion reaction buffer, 100 ng of linearized plasmid, 50-200 ng of insert, 1 unit of In-fusion enzyme, which is incubated first at 37° C. for 15 minutes and then at 50° C. for 15 minutes. The complete expression vector is transformed into XL1-Blue competent cells via standard heat shock methods. The transformed cells are selected by growth on LB-Ampicillin plates, individual colonies are used to seed 5 mL LB-Ampicillin liquid cultures and grown overnight at 37° C. and the resulting cultures are used to prepare purified plasmid stocks using standard methods. Successful cloning of the PCR product into the plasmid vector can be confirmed with restriction mapping using enzymes with sites flanking the insertion point and with PCR using primers specific to the insert sequence.

Yeast cells are transformed with expression vectors using standard methods with chemically competent yeast. Transformed cells are subjected to auxotrophic selection and resulting yeast colonies are used to seed liquid cultures, also with auxotrophic selection to maintain the expression plasmid. Liquid cultures are grown to a final density optimal for exosome production for each yeast strain. Cells are removed from the media using centrifugation and/or filtration. Extracellular vesicles containing the expressed biologically active RNA are then concentrated by precipitation with polyethylene glycol and purified by size exclusion chromatography. Vesicles are excluded from the pores of the resin and move in the void volume of the column eluting in early fractions, while free protein and co-precipitating PEG are retained by the resin and elute in later fractions. Vesicle-containing fractions are collected and pooled for use directly from the column or the vesicles can be further concentrated using centrifugal filtering devices.

FIG. 11 shows the results of a size exclusion column fractionation of extracellular vesicles obtained from a culture of Candida glabrata. Vesicles were precipitated from media conditioned for 48 hours by C. glabrata using polyethylene glycol. The precipitated vesicle fraction was resuspended in PBS and loaded to the top of a size exclusion column (Sepharose CL-6B) also equilibrated in PBS at 4° C. Fractions (1 mL) were collected and absorbance readings were taken at 260 nm (background subtractions with average reading from 350 to 450 nm) to construct the column profiles shown. The peak vesicle fraction, fraction 8, was then reloaded to the column and the process was repeated. The purified vesicles again run in the void volume fractions with no free protein present in later fractions (light grey squares).

Mammalian target cells growing in cell culture can be transfected using the purified, RNA-loaded vesicles isolated above. Target cells are plated at a cell density appropriate for the cell type and plating vessel being used (for example, ranging from 10,000 to 200,000 cells per well for a standard 12-well plate) and grown under appropriate conditions overnight (37° C., 5% CO2, 95% humidity). Transfection with the purified yeast extracellular vesicles can be carried out in media with or without growth serum, depending on the source of the vesicles. In the case of transfections done without serum, media replacement or serum addition is necessary at 6 hours post-transfection in order to maintain cell viability.

### Example 2

### Preparation of Yeast Vesicles Loaded with an Endogenously Derived Yeast Autonomous Cytoplasmic Linear DNA

Autonomously replicating yeast cytoplasmic linear plasmids are constructed from isolated plasmid backbones and PCR amplified expression cassettes for the biologically active component. In addition to the proteins encoded by the linear plasmids, which facilitate cytoplasmic replication and gene expression, the expression vector will include a promoter and terminator for expression of the RNA, both of which are appropriate for expression in the mammalian target cells. Examples of suitable linear plasmid backbones include pGKL1 and pGKL2 from Kluyveromyces lactis, pPEII and pPEIB from Pichia etchellsii, pSKL from Saccharomyces kluyveri, pDHIB from Debaryomyces hansenii, pWR1B from Wingea robertsiae, pPac1-1 from Pichia acacia, as well as pPP1 and pPP2 from Pichia pastoris. These plasmid backbones contain elements necessary to maintain the linear plasmids as extra-chromosomal elements in the yeast cytoplasm including terminal inverted repeats, an autonomous replication sequence and ORFs encoding for DNA polymerases, RNA polymerases, capping enzymes, single stranded DNA binding proteins, helicases, terminal proteins and terminal recognition factors. Some vectors may also include an expression cassette for an auxotrophic selectable marker driven by a yeast promoter from the linear plasmid to allow growth in knockout media.

Expression cassettes for the biologically active RNA are prepared by annealing DNA oligos, in the case of small RNAs, or by PCR amplification of the relevant sequences from cDNA clones, in the case of mRNA transcripts, using the appropriate forward and reverse primers as described in Example 1. Annealed oligos or PCR products are subcloned into backbone vectors carrying promoters and terminators appropriate for expression in mammalian target cells, where transcription will occur in the nucleus of the target cell. Examples of suitable backbone vectors include those derived from pCI, pET, pSI, pcDNA, pCMV, etc. Alternatively, the annealed oligos or PCR products can be subcloned behind the UCS elements derived from the open reading frames from any linear DNA vector, where transcription will occur in the cytoplasm of the target cell. Subcloning (ligation or recombination), vector preparation and validation can be performed as described in Example 1. This purified vector then serves as template for a second round of PCR using primers that flank the mammalian promoter and terminator sequences and may incorporate an optional DNA nuclear-targeting sequence (DTS, such as the one recognized by NF-κB) to generate a second expression cassette for subcloning into the yeast linear plasmid. Primers are complementary to the regions upstream of the mammalian promoter and downstream of the mammalian polyA addition sequence, they include sites for the restriction enzymes used in the subcloning as well as 15 bases of vector sequence flanking the restriction site for use in the recombination cloning. The complete expression vector is transformed into yeast cells via standard methods. The transformed cells are grown first on YPD plates, then in 5 mL liquid cultures at 30° C. The resulting cultures are used to prepare purified plasmid stocks using standard methods. Yeast cells transformed with expression vectors carrying auxotrophic selection markers grown on plates and liquid cultures containing knockout media to maintain the linear expression plasmid. Successful cloning of the PCR product into the linear plasmid vector can be confirmed with restriction mapping using enzymes with sites flanking the insertion point and with PCR using primers specific to the insert sequence.

Yeast cultures are prepared and loaded yeast extracellular vesicles are purified as described in Example 1. Loaded vesicles are then added to mammalian target cells in growth media (with or without serum) for transfection as described in Example 1.

### Example 3

### Preparation of Yeast Vesicles Loaded with Endogenously Produced Circular RNA for Delivery to Mammalian Cells

Expression vectors for the endogenously produced circular RNA are constructed from isolated plasmid backbones and PCR amplified expression cassettes for the biologically active RNA as described in Example 1. The expression vector should include at least the following components: an origin of replication for preparation in bacteria, an antibiotic selectable marker for selection in bacteria, an origin of replication for propagation in yeast, a promoter and terminator for expression of the RNA, both of which are appropriate for the yeast strain being used, as well as sequences that direct the formation of the circular RNA. Expression cassettes for the biologically active RNA or the mRNA transcript encoding the biologically active polypeptide are prepared by annealing DNA oligos, in the case of small RNAs, or by PCR amplification of the relevant sequences from cDNA clones, in the case of mRNA transcripts, using the appropriate forward and reverse primers as described in Example 1. For the final expression cassette, this PCR product is subcloned in between the sequence elements that direct formation of the circular RNA, which in turn are located between yeast promoter and terminator sequences. In the case of mRNA transcripts, the expression cassette may also include an optional poly-adenosine tract after the mRNA coding sequence but before the yeast terminator. Yeast cells are transformed with expression vectors using standard methods and extracellular vesicles containing the expressed biologically active circular RNA are then concentrated by precipitation with polyethylene glycol and purified by size exclusion chromatography. Loaded vesicles are then added to mammalian target cells in growth media (with or without serum) for transfection as described in Example 1.

### Example 4

### Preparation of Yeast Vesicles Loaded with an Endogenously Derived Yeast Autonomous Cytoplasmic Linear DNA Expressing Circular RNAs

Autonomously replicating yeast cytoplasmic linear plasmids are constructed from isolated plasmid backbones and PCR amplified expression cassettes for the biologically active component as described in Example 2. In addition to the proteins encoded by the linear plasmids, which facilitate cytoplasmic replication and gene expression, the expression vector will include a promoter and terminator for expression of the RNA, both of which are appropriate for expression in the mammalian target cells, as well as sequences that direct the formation of the circular RNA. Expression cassettes for the biologically active RNA are prepared by annealing DNA oligos, in the case of small RNAs, or by PCR amplification of the relevant sequences from cDNA clones, in the case of mRNA transcripts, using the appropriate forward and reverse primers as described in Example 1. For the final expression cassette, this PCR product is subcloned in between the sequence elements that direct formation of the circular RNA, which in turn are located either downstream of UCS sequences for expression in the mammalian cytoplasm or between mammalian promoter and terminator sequences for transcription in the mammalian nucleus. The expression cassette may also include an optional poly-adenosine tract after the mRNA coding sequence but before the mammalian terminator. Yeast cells are transformed with expression vectors using standard methods and extracellular vesicles containing the expressed cytoplasmic linear DNA are then concentrated by precipitation with polyethylene glycol and purified by size exclusion chromatography. Loaded vesicles are then added to mammalian target cells in growth media (with or without serum) for transfection as described in Example 1. Upon delivery to the mammalian target cell, the linear DNA cargo is transcribed to produce the biologically active RNA, the circular form of the RNA is created through the activities associated with the flanking sequences and the RNA is available to function in the target cell.

### Example 5

### Preparation of Yeast Vesicles Loaded with Endogenously Produced mRNA Encoding a Secreted Protein for Delivery to Mammalian Cells

Expression vectors for the endogenously produced mRNA are constructed from isolated plasmid backbones and PCR amplified expression cassettes for the biologically active RNA as described in Example 1. The expression vector should include at least the following components: an origin of replication for preparation in bacteria, an antibiotic selectable marker for selection in bacteria, an origin of replication for propagation in yeast, a promoter and terminator for expression of the RNA, both of which are appropriate for the yeast strain being used. Expression cassettes for the biologically active mRNA transcript encoding for the secreted biologically active protein are prepared by PCR amplification of the relevant sequences from cDNA clones using the appropriate forward and reverse primers as described in Example 1. Yeast cells are transformed with expression vectors using standard methods and extracellular vesicles containing the expressed mRNA are then concentrated by precipitation with polyethylene glycol and purified by size exclusion chromatography.

RNA loaded vesicles are added to mammalian target cells in growth media (with or without serum) for transfection as described in Example 1. Upon delivery of the mRNA transcript to the target cell, the mRNA is translated to produce the biologically active protein. Secretion from the target cell follows the classical secretion pathway and is facilitated by the signal sequence found in the N-terminus of the secreted protein, which guides the protein to the ER-Golgi network through its interaction with the signal recognition particle (SRP). The ribosome elongation complex is bound to the membrane of the endoplasmic reticulum and transfer of the protein across the membrane is co-translational; the secreted protein accumulates and functions in the extracellular space.

### Example 6

### Preparation of Yeast Vesicles Loaded with Endogenously Produced Polypeptides or Proteins for Delivery to Mammalian Cells

Expression vectors for the endogenously produced polypeptides or proteins are constructed from isolated plasmid backbones and PCR amplified mRNA expression cassettes as described in Example 1. Expression cassettes producing the mRNA transcripts encoding for the biologically active polypeptide or protein are prepared by PCR amplification of the relevant sequences from cDNA clones using the appropriate forward and reverse primers as described in Example 1. Yeast cells are transformed with expression vectors using standard methods and extracellular vesicles containing the expressed biologically active polypeptide are then concentrated by precipitation with polyethylene glycol and purified by size exclusion chromatography. Loaded vesicles are then added to mammalian target cells in growth media (with or without serum) for transfection as described in Example 1.

### Example 7

### Preparation of Yeast Vesicles Loaded with Endogenously Produced Intrabodies for Delivery to Mammalian Cells

Expression vectors for the endogenously produced intrabodies are constructed from isolated plasmid backbones and PCR amplified mRNA expression cassettes as described in Example 1. Expression cassettes producing the mRNA transcripts encoding for the intrabody are prepared by PCR amplification of the relevant sequences from cDNA clones using the appropriate forward and reverse primers as described in Example 1. Yeast cells are transformed with expression vectors using standard methods and extracellular vesicles containing the expressed intrabody are then concentrated by precipitation with polyethylene glycol and purified by size exclusion chromatography. Loaded vesicles are then added to mammalian target cells in growth media (with or without serum) for transfection as described in Example 1.

### Example 8

### Preparation of Yeast Vesicles Loaded with Endogenously Produced CRISPR Complexes for Delivery to Mammalian Cells

Expression vectors for the endogenously produced crRNA guide sequence and the Cas9 protein are constructed from isolated plasmid backbones and PCR amplified mRNA expression cassettes as described in Example 1. Expression cassettes producing the crRNA guides sequence and mRNA transcripts encoding for the Cas9 protein are prepared by PCR amplification of the relevant sequences from cDNA clones using the appropriate forward and reverse primers as described in Example 1. Yeast cells are transformed with expression vectors using standard methods; the crRNA guide sequence and the Cas9 protein are expressed and form a complex in the yeast cytoplasm. Extracellular vesicles containing the expressed crRNA/Cas9 complex are then concentrated by precipitation with polyethylene glycol and purified by size exclusion chromatography. Loaded vesicles are then added to mammalian target cells in growth media (with or without serum) for transfection as described in Example 1.

### Example 9

### Preparation of Yeast Vesicles Carrying Transmembrane Immune Masking Peptides and Loaded with Endogenously Produced Biologically Active RNA Molecules for Targeted Delivery to Mammalian Cells

Expression vectors for the biologically active RNA and transmembrane immune masking peptides are constructed from isolated plasmid backbones and PCR amplified mRNA expression cassettes as described in Example 1. Expression cassettes for the biologically active RNA are prepared by annealing DNA oligos, in the case of small RNAs, or by PCR amplification of the relevant sequences from cDNA clones, in the case of mRNA transcripts, using the appropriate forward and reverse primers as described in Example 1. Expression cassettes producing the transmembrane immune masking fusion protein are prepared by PCR amplification of the relevant sequences from cDNA clones using the appropriate forward and reverse primers.

Yeast cells are transformed with expression vectors using standard methods and extracellular vesicles containing the expressed biologically active polypeptide are then concentrated by precipitation with polyethylene glycol and purified by size exclusion chromatography. Loaded vesicles are then added to mammalian target cells in growth media (with or without serum) for transfection as described in Example 1.

### Example 10

### Preparation of Yeast Vesicles Carrying Transmembrane Targeting Ligands and Loaded with Endogenously Produced Biologically Active RNA Molecules for Targeted Delivery to Mammalian Cells

Expression vectors for the endogenously produced polypeptides and transmembrane targeting peptides are constructed from isolated plasmid backbones and PCR amplified mRNA expression cassettes as described in Example 1. Expression cassettes for the biologically active RNA are prepared by annealing DNA oligos, in the case of small RNAs, or by PCR amplification of the relevant sequences from cDNA clones, in the case of mRNA transcripts, using the appropriate forward and reverse primers as described in Example 1. Expression cassettes producing the transmembrane immune masking fusion protein are prepared by PCR amplification of the relevant sequences from cDNA clones using the appropriate forward and reverse primers and described in Example 4. Yeast cells are transformed with expression vectors using standard methods and extracellular vesicles containing the expressed biologically active polypeptide are then concentrated by precipitation with polyethylene glycol and purified by size exclusion chromatography. Loaded vesicles are then added to mammalian target cells in growth media (with or without serum) for transfection as described in Example 1.

### Example 11

### Preparation of Yeast Vesicles Carrying Transmembrane Immune Masking Peptides and Transmembrane Targeting Ligands Loaded with Endogenously Produced Biologically Active RNA Molecules for Targeted Delivery to Mammalian Cells

Expression vectors for the endogenously produced polypeptides, transmembrane immune masking peptides and transmembrane targeting peptides are constructed from isolated plasmid backbones and PCR amplified mRNA expression cassettes as described in Example 1. Expression cassettes for the biologically active RNA are prepared by annealing DNA oligos, in the case of small RNAs, or by PCR amplification of the relevant sequences from cDNA clones, in the case of mRNA transcripts, using the appropriate forward and reverse primers as described in Example 1. Expression cassettes producing the transmembrane immune masking fusion protein are prepared by PCR amplification of the relevant sequences from cDNA clones using the appropriate forward and reverse primers and described in Example 4. Yeast cells are transformed with expression vectors using standard methods and extracellular vesicles containing the expressed biologically active polypeptide are then concentrated by precipitation with polyethylene glycol and purified by size exclusion chromatography. Loaded vesicles are then added to mammalian target cells in growth media (with or without serum) for transfection as described in Example 1.

### Example 12

### Assays for Confirming Expression of Biologically Active RNA and Loading to Yeast Extracellular Vesicles

Yeast cells are transformed with an RNA expression vector using the methods described in Examples 1-6. Successful generation of the vesicle-producing yeast is confirmed by assays that verify one or more of the following: (1) expression of the biologically active RNA, (2) loading of the RNA to a yeast vesicle and (3) secretion of the vesicle loaded with the RNA. To detect expression of the plasmid-derived biologically active RNA, total RNA is prepared from transformed yeast cells using Qiagen's RNEasy kit according to the manufacturer's recommended protocol. A cDNA library is prepared from the total RNA using a poly-T primer and used as template for the PCR amplification. Primers for two separate amplification reactions, each producing a different size product, are included in the PCR reactions: (1) Primers amplifying sequences from an internal control gene, such as β-actin or GAPDH, and (2) Primers amplifying sequences specific to the biologically active RNA. Products are resolved on 2% agarose gels run in 1×TAE or on 10% acrylamide gels run in 1×TBE. Products are compared for the non-transfected cells (negative control) and cells transfected with a null vector (backbone vector without the biologically active RNA) through staining with ethidium bromide and illumination with UV light at 302 nm. Alternatively, primer/probe combinations specific for each target can be used in qPCR assays to detect and quantify the expressed RNA.

Successful production of the biologically active RNA molecule for loading to extracellular vesicles includes transcription of the RNA, export of that RNA from the nucleus to the cytoplasm, and uptake into vesicles. RT-PCR assays are used to show production of the plasmid-derived biologically active RNA molecule and cellular fractionation is used to demonstrate accumulation of the RNA in the cytoplasm. A cDNA library is prepared from the fractionated RNA using a random hexamer non-specific primer and is used as template for the PCR amplification. Primer/probe combinations specific for each target can be used in qPCR assays to detect and quantify the expressed RNA. Secretion of the biologically active RNA in yeast vesicles is verified by detection of the RNA in the growth media or in purified yeast vesicles.

FIG. 16 shows the results of an experiment to confirm the secretion of a GLP-1 reporter RNA from a yeast cell via an extracellular vesicle. Extracellular vesicles were purified from growth media conditioned by yeast cells transformed with plasmids expressing the GLP-1 RNA 48 hours after transformation. Total RNA was collected from both vesicles and cells using Qiagen's RNEasy kit according to the manufacturer's recommended protocol. The purified RNA was used as template for cDNA synthesis and qPCR amplification reactions. FIG. 16 shows strong expression of the GLP-1 RNA in the yeast cells as well as accumulation of that RNA in the purified vesicle fraction.

### Example 13

### Assays for Confirming Expression of Biologically Active Protein and Loading to Yeast Extracellular Vesicles

Yeast cells are transformed with an expression vector encoding for a biologically active protein using the methods described in Examples 1-6. Successful generation of the vesicle-producing yeast is confirmed by assays that verify one or more of the following: (1) expression of the biologically active protein, (2) loading of the protein to a yeast vesicle and (3) secretion of the vesicle loaded with the protein. To detect expression of the plasmid-derived biologically active protein, total protein is collected from transformed yeast cells by boiling cell lysis in SDS buffer. Total protein is concentrated from each sample by acetone precipitation and the concentrated proteins are resuspended in either a native buffer for ELISA analysis or denaturing buffer for western blot analysis. Each assay utilizes standard methods and antibodies specific for an internal control gene (β-actin or GAPDH) and a protein tag present in the biologically active protein. Non-transfected and null vector-transfected control cells have a single protein detected for the internal control gene while successful protein expressing cells have both the internal control protein and the biologically active protein.

FIG. 17 shows the results of an experiment to confirm the secretion of an Enolase reporter protein from a yeast cell via an extracellular vesicle. Extracellular vesicles were purified from growth media conditioned by yeast cells transformed with plasmids expressing the Enolase protein 48 hours after transformation. Vesicles were fractionated on a size exclusion column and total protein was precipitated from each column fractions using trichloroacetic acid (TCA). Total protein collected from precipitated column fractions and vesicle-producing cells were treated with SDS loading buffer and run on 4-20% TRIS-glycine SDS PAGE gels in TRIS-glycine buffer. Proteins resolved on the gel were then transferred to PDVF membranes for western blot analysis. Western blots were developed using antibodies specific for the streptavidin epitope tag carried by the expressed Enolase protein. FIG. 17 shows strong Enolase signals in vesicle fractions, consistent with protein loading to the yeast vesicles.

### Example 14

### Assays for Determining the Relative Output of Yeast Extracellular Vesicles from Various Yeast Strains

Various strains of yeast are cultured under similar conditions to determine which strain produces the greatest number of vesicles. Liquid cultures are grown to an equivalent cell density (as judged by OD600 measurements) and cells are removed from the media using centrifugation and/or filtration. Extracellular vesicles are then concentrated by precipitation with polyethylene glycol and purified by size exclusion chromatography. Column profiles are constructed from A260 readings of nucleic acid or A280 readings of protein (as in FIGS. 11, 13-15, and 17) and total vesicle content is determined from the area under the peak moving in the void volume. Alternatively, vesicle numbers can be compared through nanoparticle counting using nanoparticle tracking analysis available in Nanosight instruments (Malvern).

### Example 15

### Assays for Determining Relative Loading of Biologically Active RNA to Yeast Extracellular Vesicles from Various Yeast Strains

Various yeast strains are transformed with an RNA expression vector using the methods described in Examples 1-6. Liquid cultures are grown to an equivalent cell density (as judged by OD600 measurements) and cells are removed from the media using centrifugation and/or filtration. Extracellular vesicles are then concentrated by precipitation with polyethylene glycol and purified by size exclusion chromatography. Column profiles are constructed from A260 and A280 readings (as in FIGS. 11, 13-15, and 17) and total vesicle content is determined from the area under the peak moving in the void volume. These calculations are then used to normalize the vesicle concentrations across samples from the various yeast strains.

RNA loading to the yeast vesicles is quantified using qPCR. Total RNA is prepared from both the vesicle-producing yeast cells and the purified vesicles using Qiagen's RNEasy kit according to the manufacturer's recommended protocol. A cDNA sample is prepared from the total RNA using a poly-T primer and used as template for the PCR amplification. Primer/probe combinations specific for each target are used in to detect and quantify the expressed RNA in cell and vesicle samples. RNA loading efficiency can be compared in terms of relative loading percentages (fraction of total RNA found in vesicles) or as mass amounts loaded per volume of growth culture.

### Example 16

### Assays for Determining Cytotoxicity and Immune Responses Upon Administration of Yeast Extracellular Vesicles to Mammalian Target Cells In Vitro

This example describes an exemplary transfection assay to determine the immune response by a mammalian cell upon treatment with a purified stock of yeast extracellular vesicles. A number of different yeast strains are cultured to generate vesicle-producing cells as described in Examples 1-6. Yeast vesicles are collected from conditioned growth media and concentrated by precipitation with polyethylene glycol and purified by size exclusion chromatography as described in Example 1. Mammalian macrophage cells (THP-1 human macrophages or RAW264.7 mouse macrophages) are cultured separately (12-well plates, 37° C., 5% CO2, 95% humidity) for 24 hours. The purified vesicles are transferred to the macrophage cells and cultures are incubated at 37° C. for 16 hours. Media is collected from the macrophage cells and secreted cytokines (TNF-α, IFN-γ, etc.) are detected using ELISAs specific for either the human or the mouse versions of each cytokine. Responses for vesicles from different yeast strains are compared to each other and to a panel of positive and negative controls in order to grade immune responses for each.

### Example 17

### Assays for Determining Immune Responses Upon Administration of Yeast Extracellular Vesicles In Vivo

This example describes an exemplary transfection assay to determine the immune response by a mammalian cell upon treatment with a purified stock of yeast extracellular vesicles. A number of different yeast strains are cultured to generate vesicle-producing cells as described in Examples 1-6. Cells are grown for 48 hours and collected by centrifugation. Cell pellets are resuspended in phosphate buffered saline (PBS) and administered to mice through tail vein injection (dosing and schedule varied throughout experiment). Blood tissue samples are collected from mice at multiple time points and secreted cytokines (TNF-α, IFN-γ, etc.) are detected using ELISAs specific for each mouse cytokine. Alternatively, immune responses can be assessed using cytokine arrays that assess expression of many immune responsive genes simultaneously. Responses for different vesicle-producing yeast strains are compared to each other and to a panel of positive and negative controls in order to grade immune responses for each.

### Example 18

### Assays for Determining Activity of Biologically Active RNA Upon Delivery to Mammalian Target Cells In Vitro by Yeast Extracellular Vesicles

This example describes an exemplary transfection assay to determine the activity of an inhibitory shRNA delivered to the cytoplasm of a target cell by a yeast extracellular vesicle. An expression plasmid for the shRNA is transformed into a number of different yeast strains in vitro to generate vesicle-producing cells as described in Examples 1-6. Target cells expressing the mRNA transcript targeted by the shRNA are cultured separately. After 48 hours, cell media is collected from the vesicle-producing cells and vesicles are purified by size exclusion chromatography as described in Example 1. The purified vesicles are transferred to the target cells and cultures are incubated at 37° C. for 24-72 hours. Controls include addition of media from non-transformed cells and media from yeast cells transformed with empty vectors. Total RNA is prepared from the target cells and RT-PCR analysis is carried out as described in Example 7. Knockdown of the target gene is assessed by comparison to a non-targeted internal control gene. Alternatively, vesicle-producing yeast cells and target cells can be cultured together during the experiment, since the primers and probes used in the RT-PCR assays will not recognize the corresponding transcripts in the yeast cells. In this case, yeast cells can be collected 24 hours after transformation and mixed with target cells for direct assays of bioreactor activity as assayed by RT-PCR analysis. The delivery of other shRNAs by yeast vesicles can be assayed using similar methods with the appropriate target cells.

### Example 19

### In Vivo Administration of Purified Yeast Extracellular Vesicles Loaded with Biologically Active RNA to Mice

Yeast cells are transformed with an RNA expression vector using the methods described in Examples 1-6. Loading of RNA to vesicles is verified with the assays described in Example 7. Isolation, concentration and purification of the yeast vesicles are as described in Example 1. In this example, the yeast cells produce vesicles carrying an shRNA targeting the c-Myc transcript. The purified vesicles are mixed with SCCVII target cells (a mouse squamous cell carcinoma line) and the mixture is transplanted into nude mice (immune-compromised) by subcutaneous injection into the rear flanks of each animal. Activity is monitored by assessment of c-Myc transcript and protein levels in tissues surrounding the transplantation site. RNA samples are prepared from tissue collected from the rear flanks of untreated mice, mice transplanted with SCCVII cells alone and mice transplanted with SCCVII cells mixed with vesicles carrying the shRNAs targeting c-Myc using Tri-Reagent (Sigma-Aldrich, product # T9424). Relative levels of c-Myc transcript can then be assessed by RT-PCR as described in Example 7. c-Myc target gene knockdown and its impact on SCCVII cell viability will also be assessed in vivo by comparing tumor growth in the shRNA vesicle/SCCVII transplants to control mice receiving SCCVII cells alone or SCCVII cells with vesicles carrying non-specific shRNAs.

### Example 20

### In Vivo Administration of Yeast Cells Producing Extracellular Vesicles Loaded with Biologically Active RNA to Mice

Yeast cells are transformed with an RNA expression vector using the methods described in Examples 1-6. Loading of RNA to vesicles is verified with the assays described in Example 7. In this example, the yeast cells produce vesicles carrying an shRNA targeting the c-Myc transcript. The vesicle-producing yeast cells are mixed with SCCVII target cells (a mouse squamous cell carcinoma line) and the mixture is transplanted into nude mice (immune-compromised) by subcutaneous injection into the rear flanks of each animal. Activity is monitored by assessment of c-Myc transcript and protein levels in tissues surrounding the transplantation site. RNA samples are prepared from tissue collected from the rear flanks of untreated mice, mice transplanted with SCCVII cells alone and mice transplanted with SCCVII cells mixed with vesicle-producing cells carrying the shRNAs targeting c-Myc using Tri-Reagent (Sigma-Aldrich, product # T9424). Relative levels of c-Myc transcript can then be assessed by RT-PCR as described in Example 7. c-Myc target gene knockdown and its impact on SCCVII cell viability will also be assessed in vivo by comparing tumor growth in the shRNA vesicle/SCCVII transplants to control mice receiving SCCVII cells alone or SCCVII cells with vesicles carrying non-specific shRNAs.

### Example 21

### In Vivo Administration of Purified Yeast Extracellular Vesicles Loaded with a Circular miRNA Sponge to Mice

Yeast cells are transformed with an mRNA expression vector encoding for a circular miRNA sponge using the methods described in Examples 1-6. After transcription in the yeast nucleus, the miRNA sponge is circularized by the RNA ribozyme sequences and exported to the cytoplasm. This circular RNA is then loaded to the yeast extracellular vesicle. Loading of the RNA is verified with the assays described in Example 8. Isolation, concentration and purification of the yeast vesicles are as described in Example 1. In this example, the yeast cells produce vesicles carrying a circular miRNA sponge carrying target sequences for the miR-183 microRNA. Cell media is collected 72 hours after transformation and vesicles are purified by size exclusion chromatography as described in Example 1. These vesicles will be used to treat a subcutaneous model of human prostate cancer. The vesicle-producing yeast cells are mixed with PC-3, DU-145, or LNCaP target cells (human prostate cancer cell lines, PC) and the mixture is transplanted into nude mice (immune-compromised) by subcutaneous injection into the rear flanks of each animal. Activity of the miRNA sponges is evaluated by comparing tumor growth in the miRNA vesicle/PC cell transplants to control mice receiving PC cells alone or PC cells with vesicles carrying non-specific miRNA sponges.

### Example 22

### Systemic Administration of Purified Yeast Extracellular Vesicles Loaded with Biologically Active RNA to Mice Via Tail Vein Injection

Yeast cells are transformed with an RNA expression vector using the methods described in Examples 1-6. Loading of RNA to vesicles is verified with the assays described in Example 7. Isolation, concentration and purification of the yeast vesicles are as described in Example 1. In this example, the yeast cells produce vesicles carrying an mRNA transcript encoding for the human p53 protein. Cell media is collected 48 hours after transformation and vesicles are purified by size exclusion chromatography as described in Example 1. These vesicles will be used to treat a therapeutic xenograft model of human lung metastatic cancer, generated by administering either H1299 or A549 tumor cells to immunodeficient mice (SCID mice or nu/nu mice) by tail vein injection. The purified vesicles loaded with p53 transcript are then administered to mice by tail vein injection (dose and schedule varied throughout study). Activity of the vesicles is evaluated by measurement of tumor volume, tumor number and overall survival. In vivo assessments will be compared to control mice receiving no vesicles and mice receiving vesicles carrying a scrambled mRNA transcript.

### Example 23

### Systemic Administration of Purified Yeast Extracellular Vesicles Loaded with a Circular mRNA Transcript to Mice Via Tail Vein Injection

Yeast cells are transformed with an mRNA expression vector encoding for a circular mRNA transcript using the methods described in Examples 1-6. After transcription in the yeast nucleus, the mRNA is circularized by the RNA ribozyme sequences and exported to the cytoplasm. This circular RNA is then loaded to the yeast extracellular vesicle. Loading of the RNA is verified with the assays described in Example 8. Isolation, concentration and purification of the yeast vesicles are as described in Example 1. In this example, the yeast cells produce vesicles carrying a circular mRNA transcript encoding the p53 tumor suppressor protein. Cell media is collected 72 hours after transformation and vesicles are purified by size exclusion chromatography as described in Example 1. These vesicles will be used to treat a therapeutic xenograft model of human lung metastatic cancer, generated by administering either H1299 or A549 tumor cells to immunodeficient mice (SCID mice or nu/nu mice) by tail vein injection. The purified vesicles loaded with the circular p53 mRNA are administered to mice by tail vein injection (dose and schedule varied throughout study). Activity of the vesicles is evaluated by measurement of tumor growth and overall survival. In vivo assessments will be compared to control mice receiving no vesicles and mice receiving vesicles carrying a scrambled mRNA transcript.

### Example 24

### Systemic Administration of Purified Yeast Extracellular Vesicles Loaded with Biologically Active Protein to Mice Via Tail Vein Injection

Yeast cells are transformed with an mRNA expression vector encoding for a biologically active protein using the methods described in Examples 1-6. Loading of the biologically active protein to vesicles is verified with the assays described in Example 8. Isolation, concentration and purification of the yeast vesicles are as described in Example 1. In this example, the yeast cells produce vesicles carrying the human p53 protein. Cell media is collected 48 hours after transformation and vesicles are purified by size exclusion chromatography as described in Example 1. These vesicles will be used to treat a therapeutic xenograft model of human lung metastatic cancer, generated by administering either H1299 or A549 tumor cells to immunodeficient mice (SCID mice or nu/nu mice) by tail vein injection. The purified vesicles loaded with p53 protein are then administered to mice by tail vein injection (dose and schedule varied throughout study). Activity of the vesicles is evaluated by measurement of tumor volume, tumor number and overall survival. In vivo assessments will be compared to control mice receiving no vesicles and mice receiving vesicles carrying a scrambled mRNA transcript.

### Example 25

### Systemic Administration of Purified Yeast Extracellular Vesicles Loaded with crRNA/Cas9 Complexes to Mice Via Tail Vein Injection

Yeast cells are transformed with an mRNA expression vector encoding for a crRNA guide sequence and an mRNA encoding for the Cas9 protein using the methods described in Examples 1-6. After export to the cytoplasm, the mRNA is translated to produce the Cas9 protein, which then binds to the crRNA guide sequence. This RNA-protein complex is then loaded to the yeast extracellular vesicle. Loading of the complex is verified with the assays described in Example 8. Isolation, concentration and purification of the yeast vesicles are as described in Example 1. In this example, the yeast cells produce vesicles carrying crRNA/Cas9 complexes with guide sequences targeting the mIR145 locus. Cell media is collected 72 hours after transformation and vesicles are purified by size exclusion chromatography as described in Example 1. These vesicles will be used to treat a rat model of pulmonary arterial hypertension. The purified vesicles loaded with the crRNA/Cas9 complexes are administered to mice by tail vein injection (dose and schedule varied throughout study). Activity of the vesicles is evaluated by measurement of hemodynamic parameters indicative of disease progression. In vivo assessments will be compared to control mice receiving no vesicles and mice receiving vesicles carrying a scrambled crRNA guide sequence.

### Example 26

### In Vivo Administration of Purified Yeast Extracellular Vesicles Loaded with Intrabodies to Mice Via Intra-Tumoral Injection

Yeast cells are transformed with an mRNA expression vector encoding for an intrabody using the methods described in Examples 1-6. Loading of the intrabody to vesicles is verified with the assays described in Example 8. Isolation, concentration and purification of the yeast vesicles are as described in Example 1. In this example, the yeast cells produce vesicles carrying an intrabody targeting the p21 Ras protein. Cell media is collected 72 hours after transformation and vesicles are purified by size exclusion chromatography as described in Example 1. These vesicles will be used to treat a sub-cutaneous model of human colon carcinoma, generated by administering HCT116 cells to immunodeficient mice (SCID or nu/nu). The purified vesicles loaded with the intrabody are then administered to mice by intra-tumoral injection (dose and schedule varied throughout study). Activity of the vesicles is evaluated by measurement of tumor volume. In vivo assessments will be compared to control mice receiving no vesicles and mice receiving vesicles carrying a control intrabody.

### Example 27

### In Vivo Administration of Yeast Vesicles Loaded with Biologically Active RNA to Mice Tumors Via Intra-Tumoral Injection

Yeast cells are transformed with an RNA expression vector using the methods described in Examples 1-6. Loading of RNA to vesicles is verified with the assays described in Example 7. In this example, the yeast cells produce vesicles carrying an shRNA targeting the transcript encoding the mutant KRAS protein (G12D). Cell media is collected 48 hours after transformation and vesicles are purified by size exclusion chromatography as described in Example 1. These vesicles will be used to treat a xenograft model of human pancreatic cancer using Panc1 cells constitutively expressing luciferase. A Panc1-Luc subcutaneous tumor is established by injection of log-phase growth cells into the flanks of mice. Established tumors will have an average volume of 80 mm³. The purified vesicles are then injected into the established Panc1 tumors (dose and schedule varied throughout study) and activity of the shRNA loaded vesicles is evaluated by in vivo imaging of luciferase expression and overall survival. In vivo assessments will be compared to control mice receiving no vesicles and mice receiving vesicles carrying non-specific shRNAs.

### Example 28

### In Vivo Administration of Yeast Cells Producing Extracellular Vesicles Loaded with Biologically Active RNA to Mice Tumors Via Intra-Tumoral Injection

Yeast cells are transformed with an RNA expression vector using the methods described in Examples 1-6. Loading of RNA to vesicles is verified with the assays described in Example 7. In this example, the yeast cells produce vesicles carrying an shRNA targeting the transcript encoding the mutant KRAS protein (G12D). Cells are grown for 48 hours and collected by centrifugation. Cell pellets are resuspended in phosphate buffered saline (PBS). These vesicle-producing cells will be used to treat a xenograft model of human pancreatic cancer using Panel cells constitutively expressing luciferase. A Panc1-Luc subcutaneous tumor is established by injection of log-phase growth cells into the flanks of mice. Established tumors will have an average volume of 80 mm³. The vesicle-producing cells are then injected into the established Panc1 tumors (dose and schedule varied throughout study) and activity of the shRNA loaded vesicles is evaluated by in vivo imaging of luciferase expression and overall survival. In vivo assessments will be compared to control mice receiving no cells and mice receiving vesicle-producing cells carrying non-specific shRNAs.

### Example 29

### Systemic Administration of Yeast Cells Producing Extracellular Vesicles Loaded with Biologically Active RNA to Mice Via Tail Vein Injection

Yeast cells are transformed with an RNA expression vector using the methods described in Examples 1-6. Loading of RNA to vesicles is verified with the assays described in Example 7. In this example, the yeast cells produce vesicles carrying an mRNA transcript encoding for the human p53 protein. Cells are grown for 48 hours and collected by centrifugation. Cell pellets are resuspended in phosphate buffered saline (PBS). These vesicle-producing cells will be used to treat a therapeutic xenograft model of human lung metastatic cancer, generated by administering either H1299 or A549 tumor cells to immunodeficient mice (SCID mice or nu/nu mice) by tail vein injection. The yeast cells producing vesicles loaded with p53 transcript are then administered to mice by tail vein injection (dose and schedule varied throughout study). Activity is evaluated by measurement of tumor volume, tumor number and overall survival. In vivo assessments will be compared to control mice receiving no vesicles and mice receiving vesicles carrying a scrambled mRNA transcript.

### Example 30

### Generating Mutant Yeast Strains that Produce High Levels of Extracellular Vesicles

Mutant yeast strains that produce high levels of extracellular vesicles compared to their wild type counterparts are generated by knocking out genes of interest via homologous recombination. A non-limiting example of a group of target genes that could influence vesicle production includes genes that influence cell wall biosynthesis (CWP1, CHS3, FKS1, FMP45, MNN9, PUN1, and SMI1). In this process, a DNA fragment targeting the gene of interest and encoding for a selectable marker (such as kanamycin) is created via PCR. The PCR primers used to generate this fragment amplify the selectable marker and add sequences complementary to regions upstream of the translational start site and downstream of the translational terminator for the target gene. Complementary regions are added in two rounds of PCR to produce a final product with 45 base pairs of complementary sequence at each end of the product. After purification, this PCR product is transformed into the yeast of interest and transformed cells are identified by growth on plates with the selective antibiotic. Positive transformants are identified as colonies growing under selective conditions and screened for successful deletion of the gene of interest by PCR. Vesicle secretion from successful deletion mutants are then assessed using the methods described in Example 9.

### Example 31

### Protein Expression in S. cerevisiae CHS3 Mutant Strain

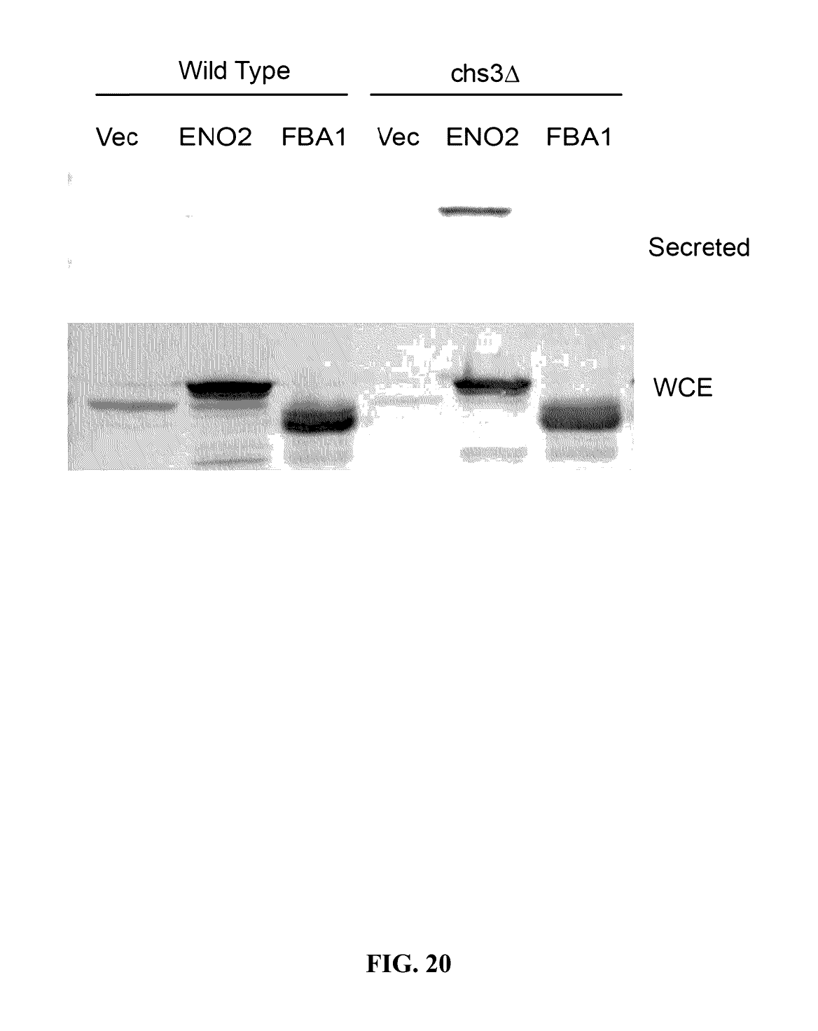

Vesicles derived from a mutant strain of yeast (S. cerevisiae chs3Δ) show an increase in the amount of the ENO2 protein compared to wild-type S. cerevisiae. This is in contrast to a second protein, FBA1, which is not increased in the vesicles. The results are shown in FIG. 20.

### Example 32

### Quantification the Amount of RNA Loaded into Yeast Vesicles

RNA loading to yeast extracellular vesicles is measured using a quantitative polymerase chain reaction (qPCR). Expression vectors for the GFP reporter RNA are constructed from isolated plasmid backbones and PCR amplified mRNA expression cassettes as described in Example 1. Yeast cells are transformed with the expression vector using the methods described in Examples 1-6. Extracellular vesicles are then concentrated by precipitation with polyethylene glycol and purified by size exclusion chromatography. Column profiles are constructed from A260 and A280 readings (as in FIGS. 11, 13-15, and 17) and total vesicle content is determined from the area under the peak moving in the void volume. Total RNA is purified from vesicles and vesicle-producing yeast cells using an RNEasy purification kit (Qiagen, product #74104). These RNA stocks serve as templates for synthesis of complementary DNAs (cDNAs) using oligo-dT primers and reverse transcriptase (Multiscribe, Applied Biosystems, cat#4311235). The reverse transcription reaction contains: 10 mM Tris (pH 8.3), 50 mM KCl, 5 mM MgCl2, 5 μg total RNA, 2.5 μM oligo dT primer, 500 μM each dNTP, 1 unit/μL RNAse inhibitor, 1 unit/mL Reverse Transcriptase. Gene specific primers and probes are used in qPCR to amplify the transcript of interest from the corresponding cDNA template (see FIG. 16). The PCR reaction contains: 10 mM Tris (pH 8.3), 50 mM KCl, 5 mM MgCl2, 5 μg total RNA, 200 nM forward and reverse primers, 200 μM each dNTP, 0.1 unit/mL Taq polymerase. All changes are measured against an internal control using primers and probes specific for a typical housekeeping gene (β-actin or GAPDH for oligo-dT primed mRNA cDNAs or 18S rRNA for random hexamer primed rRNA cDNAs). The threshold cycle (Ct value) measured for vesicle samples are compared to standard curves generated using purified RNA amplicon stocks to derive RNA concentration (pg/μL).

### Example 33

### Proteomic Analysis for S. cerevisiae CHS3 Mutant and C. glabrata

A proteomic analysis for yeast proteins identified in extracellular vesicles from two yeast strains: (1) Saccharomyces cerevisiae (Chitin synthase 3 deletion strain, chs3Δ) and (2) Candida glabrata was conducted. The 20 proteins for each strain are shown in Tables 1 (Saccharomyces cerevisiae, chs3Δ) and 2 (Candida glabrata). All of the identified proteins in the yeast vesicles had yeast specific sequences, which is distinguishable from mammalian vesicles. SEC14, TSA1, and GAS1 are proteins for which mammalian homologs have not been reported in vesicles before (according to a database of exosomal proteins, Exocarta).

TABLE 1
Proteins in mutant Saccharomyces cerevisiae vesicles

| Protein | | |
|---|---|---|
| Abundance | Gene | Function |
| 1 | ACT1 | Actin |
| 2 | TDH3 | Glyceraldehyde-3-phosphate |
| | | dehydrogenase 2 |
| 3 | ENO2 | Enolase 2 |
| 4 | ENO1 | Enolase 1 |
| 5 | FBA1 | aldolase |
| 6 | PYK1 | Pyruvate kinase 1 |
| 7 | RPS20 | ribosomal protein |
| 8 | RRP46 | ribosomal protein |
| 9 | SSB1 | Hsp70 |
| 10 | SSA3 | Hsp70 |
| 11 | HSC82 | Hsp90 |
| 12 | SEC14 | Phosphatidylinositol/phosphatidylcholine |
| | | transfer protein |
| 13 | SSA1 | Hsp70 |
| 14 | KAR2 | ER - Hsp70 |
| 15 | SSE1 | Hsp70 |
| 16 | CCT2 | CCT2 |
| 17 | ILV5 | Acetohydroxyacid reductoisomerase |
| 18 | TSA1 | Thioredoxin peroxidase |
| 19 | GAS1 | 1,3-beta-glucanosyltransferase |
| 20 | BMH2 | 14-3-3 protein |

TABLE 2
Proteins in Candida glabrata vesicles

| Abundance | Gene | Function |
|---|---|---|
| 1 | TDH3 | Glyceraldehyde-3-phosphate |
| | | dehydrogenase 2 |
| 2 | ACT1 | Actin |
| 3 | ENO1 | Enolase |
| 4 | ENO2 | Enolase 2 |
| 5 | ADH1 | Alcohol dehydrogenase |
| 6 | PMU2 | Acid phosphatase |
| 7 | PDC1 | pyruvate decarboxylase |
| 8 | CAR2 | L-ornithine transaminase |
| 9 | TSA1 | Thioredoxin peroxidase |
| 10 | PCK1 | Phosphoenolpyruvate carboxykinase |
| 11 | FBA1 | aldolase |
| 12 | SEC14 | Phosphatidylinositol/phosphatidylcholine |
| | | transfer protei |
| 13 | RRP46 | Ribosomal proteine |
| 14 | ATP2 | ATP synthase subunit |
| 15 | GAS1 | 1,3-beta-glucanosyltransferase |
| 16 | CPR5 | Peptidyl-prolyl cis-trans isomerase |
| 17 | RPL10A | Ribosomal proteine |
| 18 | THR1 | Homoserine kinase |
| 19 | PYK1 | Pyruvate kinase |
| 20 | OYE2 | NADPH oxioreductase |

### REFERENCES

1. Bozzuto, G. and A. Molinari, Liposomes as nanomedical devices. International journal of nanomedicine, 2015. 10: p. 975-99.
2. Prabhu, R. H., V. B. Patravale, and M. D. Joshi, Polymeric nanoparticles for targeted treatment in oncology: current insights. International journal of nanomedicine, 2015. 10: p. 1001-18.
3. Draz, M. S., et al., Nanoparticle-mediated systemic delivery of siRNA for treatment of cancers and viral infections. Theranostics, 2014. 4(9): p. 872-92.
4. Zhang, Y., A. Satterlee, and L. Huang, In vivo gene delivery by nonviral vectors: overcoming hurdles? Molecular therapy: the journal of the American Society of Gene Therapy, 2012. 20(7): p. 1298-304.
5. Goodwin, T. and L. Huang, Nonviral vectors: we have come a long way. Advances in genetics, 2014. 88: p. 1-12.
6. S, E. L. A., et al., Extracellular vesicles: biology and emerging therapeutic opportunities. Nature reviews. Drug discovery, 2013. 12(5): p. 347-57.
7. Nazarenko, I., A. K. Rupp, and P. Altevogt, Exosomes as a potential tool for a specific delivery of functional molecules. Methods in molecular biology, 2013. 1049: p. 495-511.
8. Marcus, M. E. and J. N. Leonard, FedExosomes: Engineering Therapeutic Biological Nanoparticles that Truly Deliver. Pharmaceuticals, 2013. 6(5): p. 659-80.
9. van der Pol, E., et al., Classification, functions, and clinical relevance of extracellular vesicles. Pharmacological reviews, 2012. 64(3): p. 676-705.
10. Wiklander, O. P., et al., Extracellular vesicle in vivo biodistribution is determined by cell source, route of administration and targeting. Journal of extracellular vesicles, 2015. 4: p. 26316.
11. Johnsen, K. B., et al., A comprehensive overview of exosomes as drug delivery vehicles—endogenous nanocarriers for targeted cancer therapy. Biochimica et biophysica acta, 2014. 1846(1): p. 75-87.
12. O'Loughlin, A. J., C. A. Woffindale, and M. J. Wood, Exosomes and the emerging field of exosome-based gene therapy. Current gene therapy, 2012. 12(4): p. 262-74.
13. Hood, J. L., M. J. Scott, and S. A. Wickline, Maximizing exosome colloidal stability following electroporation. Analytical biochemistry, 2014. 448: p. 41-9.
14. Lai, R. C., et al., Exosomes for drug delivery—a novel application for the mesenchymal stem cell. Biotechnology advances, 2013. 31(5): p. 543-51.
15. El-Andaloussi, S., et al., Exosome-mediated delivery of siRNA in vitro and in vivo. Nature protocols, 2012. 7(12): p. 2112-26.
16. Lakhal, S. and M. J. Wood, Exosome nanotechnology: an emerging paradigm shift in drug delivery: exploitation of exosome nanovesicles for systemic in vivo delivery of RNAi heralds new horizons for drug delivery across biological barriers. BioEssays: news and reviews in molecular, cellular and developmental biology, 2011.33(10): p. 737-41.
17. Zomer, A., et al., Exosomes: Fit to deliver small RNA. Communicative & integrative biology, 2010. 3(5): p. 447-50.
18. Kutralam-Muniasamy, G., L. B. Flores-Cotera, and F. Perez-Guevara, Potential of yeast secretory vesicles in biodelivery systems. Drug discovery today, 2015.
19. Vallejo, M. C., et al., Lipidomic analysis of extracellular vesicles from the pathogenic phase of Paracoccidioides brasiliensis. PloS one, 2012. 7(6): p. e39463.
20. Vallejo, M. C., et al., The pathogenic fungus Paracoccidioides brasiliensis exports extracellular vesicles containing highly immunogenic alpha-Galactosyl epitopes. Eukaryotic cell, 2011. 10(3): p. 343-51.
21. Oliveira, D. L., et al., Extracellular vesicles from Cryptococcus neoformans modulate macrophage functions. Infection and immunity, 2010. 78(4): p. 1601-9.
22. Suk, K., et al., Reconstitution of human RNA interference in budding yeast. Nucleic acids research, 2011.39(7): p. e43.
23. Nicolas, F. E., S. Torres-Martinez, and R. M. Ruiz-Vazquez, Loss and retention of RNA interference in fungi and parasites. PLoS pathogens, 2013. 9(1): p. e1003089.
24. Gunge, N. and K. Sakaguchi, Intergeneric transfer of deoxyribonucleic acid killer plasmids, pGKI1 and pGKI2, from Kluyveromyces lactis into Saccharomyces cerevisiae by cell fusion. Journal of bacteriology, 1981. 147(1): p. 155-60.
25. Gunge, N., et al., Isolation and characterization of linear deoxyribonucleic acid plasmids from Kluyveromyces lactis and the plasmid-associated killer character. Journal of bacteriology, 1981. 145(1): p. 382-90.
26. Wesolowski, M., P. Dumazert, and H. Fukuhara, Killer DNA Plasmids of the Yeast Kluyveromyces lactis: II. Restriction Endonuclease Maps. Current genetics, 1982. 5(3): p. 199-203.
27. Wesolowski, M., et al., Killer DNA Plasmids of the Yeast Kluyveromyces lactis: I. Mutations Affecting the Killer Phenotype. Current genetics, 1982. 5(3): p. 191-7.
28. Wesolowski, M., A. Algeri, and H. Fukuhara, Killer DNA plasmids of the yeast Kluyveromyces lactis: III. Plasmid recombination. Current genetics, 1982. 5(3): p. 205-8.
29. Hishinuma, F., et al., Cloning and nucleotide sequences of the linear DNA killer plasmids from yeast. Nucleic acids research, 1984. 12(19): p. 7581-97.
30. Stark, M. J., et al., Nucleotide sequence and transcription analysis of a linear DNA plasmid associated with the killer character of the yeast Kluyveromyces lactis. Nucleic acids research, 1984. 12(15): p. 6011-30.
31. Sugisaki, Y., et al., Characterization of a novel killer toxin encoded by a double-stranded linear DNA plasmid of Kluyveromyces lactis. European journal of biochemistry/FEBS, 1984. 141(2): p. 241-5.
32. Kikuchi, Y., et al., Hairpin plasmid—a novel linear DNA of perfect hairpin structure. The EMBO journal, 1985. 4(7): p. 1881-6.
33. Sakaguchi, K., H. Hirochika, and N. Gunge, Linear plasmids with terminal inverted repeats obtained from Streptomyces rochei and Kluvveromyces lactis. Basic life sciences, 1985. 30: p. 433-51.
34. Ligon, J. M., et al., Physical and biological characterization of linear DNA plasmids of the yeast Pichia inositovora. Plasmid, 1989. 21(3): p. 185-94.
35. Stark, M. J., et al., The plasmid-encoded killer system of Kluyveromyces lactis: a review. Yeast, 1990. 6(1): p. 1-29.
36. Worsham, P. L. and P. L. Bolen, Killer toxin production in Pichia acaciae is associated with linear DNA plasmids. Current genetics, 1990. 18(1): p. 77-80.
37. Bolen, P. L., et al., Physical and genetic characterization of linear DNA plasmids from the heterothallic yeast Saccharomycopsis crataegensis. Antonie van Leeuwenhoek, 1992. 61(3): p. 195-205.
38. Gunge, N., et al., Osmophilic linear plasmids from the salt-tolerant yeast Debaryomyces hansenii. Current genetics, 1993. 23(5-6): p. 443-9.
39. Bolen, P. L., et al., Isolation and sequence analysis of a gene from the linear DNA plasmid pPacl-2 of Pichia acaciae that shows similarity to a killer toxin gene of Kluyveromyces lactis. Yeast, 1994. 10(3): p. 403-14.
40. Cong, Y. S., et al., Linear DNA plasmids from Pichia etchellsii, Debaryomyces hansenii and Wingea robertsiae. Microbiology, 1994. 140 (Pt 6): p. 1327-35.
41. Schaffrath, R. and K. D. Breunig, Genetics and molecular physiology of the yeast Kluyveromyces lactis. Fungal genetics and biology: FG & B, 2000. 30(3): p. 173-90.
42. Klassen, R., et al., Genome organization of the linear Pichia etchellsii plasmid pPE1A: evidence for expression of an extracellular chitin-binding protein homologous to the alpha-subunit of the Kluyveromyces lactis killer toxin. Plasmid, 2002. 47(3): p. 224-33.
43. Klassen, R. and F. Meinhardt, Linear plasmids pWR1A and pWR1B of the yeast Wingea robertsiae are associated with a killer phenotype. Plasmid, 2002. 48(2): p. 142-8.
44. Klassen, R. and F. Meinhardt, Structural and fiminctional analysis of the killer element pPin1-3 from Pichia inositovora. Molecular genetics and genomics: MGG, 2003. 270(2): p. 190-9.
45. Jung, G. H., M. C. Leavitt, and J. Ito, Yeast killer plasmid pGKL1 encodes a DNA polymerase belonging to the family B DNA polymerases. Nucleic acids research, 1987. 15(21): p. 9088.
46. Gunge, N. and K. Kitada, Replication and maintenance of the Kluyveromyces linear pGKL plasmids. European journal of epidemiology, 1988. 4(4): p. 409-14.
47. Wilson, D. W. and P. A. Meacock, Extranuclear gene expression in yeast: evidence for a plasmid-encoded RNA polymerase of unique structure. Nucleic acids research, 1988. 16(16): p. 8097-112.
48. Schaffrath, R., S. M. Soond, and P. A. Meacock, The DNA and RNA polymerase genes of yeast plasmid pGKL2 are essential loci for plasmid integrity and maintenance. Microbiology, 1995. 141 (Pt 10): p. 2591-9.
49. Fukuda, K., et al., The linear plasmid pDHL1 from Debaryomyces hansenii encodes a protein highly homologous to the pGKL1-plasmid DNA polymerase. Yeast, 1997. 13(7): p. 613-20.
50. Schaffrath, R., F. Meinhardt, and P. A. Meacock, ORF7 of yeast plasmid pGKL2: analysis ofgene expression in vivo. Current genetics, 1997. 31(2): p. 190-2.
51. van Erp, P. B., et al., The history and market impact of CRISPR RNA-guided nucleases. Current opinion in virology, 2015. 12: p. 85-90.
52. Ain, Q. U., J. Y. Chung, and Y. H. Kim, Current and future delivery systems for engineered nucleases: ZFN, TALEN and RGEN. Journal of controlled release: official journal of the Controlled Release Society, 2015. 205: p. 120-127.
53. Doudna, J. A. and E. Charpentier, Genome editing. The new frontier of genome engineering with CRISPR-Cas9. Science, 2014. 346(6213): p. 1258096.
54. Gupta, R. M. and K. Musunuru, Expanding the genetic editing tool kit: ZFNs. TALENs, and CRISPR-Cas9. The Journal of clinical investigation, 2014. 124(10): p. 4154-61.
55. van der Oost, J., et al., Unravelling the structural and mechanistic basis of CRISPR-Cas systems. Nature reviews. Microbiology, 2014. 12(7): p. 479-92.
56. Hsu, P. D., E. S. Lander, and F. Zhang, Development and applications of CRISPR-Ca9 for genome engineering. Cell, 2014. 157(6): p. 1262-78.
57. Wiedenheft, B., S. H. Sternberg, and J. A. Doudna, RNA-guided genetic silencing systems in bacteria and archaea. Nature, 2012. 482(7385): p. 331-8.
58. Kaiser, P. D., et al., Recent progress in generating intracellular functional antibody fragments to target and trace cellular components in living cells. Biochimica et biophysica acta, 2014. 1844(11): p. 1933-1942.
59. Teillaud, J. L., From whole monoclonal antibodies to single domain antibodies: think small. Methods in molecular biology, 2012. 911: p. 3-13.
60. Oliveira, D. L., et al., Characterization of yeast extracellular vesicles: evidence for the participation of different pathways of cellular traffic in vesicle biogenesis. PloS one, 2010. 5(6): p. e11113.
61. Oliveira, D. L., et al., Biogenesis of extracellular vesicles in yeast: Many questions with few answers. Communicative & integrative biology, 2010. 3(6): p. 533-5.
62. Cocucci, E. and J. Meldolesi, Ectosomes and exosomes: shedding the confusion between extracellular vesicles. Trends in cell biology, 2015.
63. Choi, D. S., et al., Proteomics of extracellular vesicles: Exosomes and ectosomes. Mass spectrometry reviews, 2014.
64. Choi, D. S., et al., Proteomics, transcriptomics and lipidomics of exosomes and ectosomes. Proteomics, 2013. 13(10-11): p. 1554-71.
65. Nouri, F. S., et al., Reducing the Visibility of the Vector DNA Nanocomplexes to the Immune System by Elastin-Like Peptides. Pharmaceutical research, 2015.
66. Wu, C., et al., Mapping the binding between the tetraspanin molecule (Sjc23) of Schistosoma japonicum and human non-immune IgG. PloS one, 2011. 6(4): p. e19112.
67. Lysakova-Devine, T., et al., Viral inhibitory peptide of TLR4, a peptide derived from vaccinia protein A46, specifically inhibits TLR4 by directly targeting MyD88 adaptor-like and TRIF-related adaptor molecule. Journal of immunology, 2010. 185(7): p. 4261-71.
68. Carrion Sde, J., et al., The RodA hydrophobin on Aspergillus fumigatus spores masks dectin-1- and dectin-2-dependent responses and enhances fungal survival in vivo. Journal of immunology, 2013. 191(5): p. 2581-8.
69. Mei, L., et al., Increased tumor targeted delivery using a multistage liposome system functionalized with RGD, TAT and cleavable PEG. International journal of pharmaceutics, 2014. 468(1-2): p. 26-38.
70. Yang, Y., et al., Dual-modified liposomes with a two-photon-sensitive cell penetrating peptide and NGR ligand for siRNA targeting delivery. Biomaterials, 2015. 48: p. 84-96.
71. Yang, Y., et al., PEGylated liposomes with NGR ligand and heat-activable cell-penetrating peptide-doxorubicin conjugate for tumor-specific therapy. Biomaterials, 2014. 35(14): p. 4368-81.
72. Takara, K., et al., Size-controlled, dual-ligand modified liposomes that target the tumor vasculature show promise for use in drug-resistant cancer therapy. Journal of controlled release: official journal of the Controlled Release Society, 2012. 162(1): p. 225-32.
73. Dunne, M., et al., APN/CD13-targeting as a strategy to alter the tumor accumulation of liposomes. Journal of controlled release: official journal of the Controlled Release Society, 2011. 154(3): p. 298-305.
74. Li, M., et al., Targeted delivery of cisplatin by LHRH-peptide conjugated dextran nanoparticles suppresses breast cancer growth and metastasis. Acta biomaterialia, 2015. 18: p. 132-43.
75. Kim, S. H., et al., LHRH receptor-mediated delivery of siRNA using polyelectrolyte complex micelles self-assembled from siRNA-PEG-LHRH conjugate and PEI. Bioconjugate chemistry, 2008. 19(11): p. 2156-62.
76. Dharap, S. S., et al., Tumor-specific targeting of an anticancer drug delivery system by LHRH peptide. Proceedings of the National Academy of Sciences of the United States of America, 2005. 102(36): p. 12962-7.
77. Snyder, E. L., et al., Enhanced targeting and killing of tumor cells expressing the CXC chemokine receptor 4 by transducible anticancer peptides. Cancer research, 2005. 65(23): p. 10646-50.
78. Suzuki, H. and T. Tsukahara, A view of pre-mRNA splicing from RNase R resistant RNAs. International journal of molecular sciences, 2014. 15(6): p. 9331-42.
79. Lasda, E. and R. Parker, Circular RNAs: diversity of form and function. RNA, 2014. 20(12): p. 1829-42.
80. Chen, L. L. and L. Yang, Regulation of circRNA biogenesis. RNA biology, 2015. 12(4): p. 381-8.
81. Ford, E. and M. Ares, Jr., Synthesis of circular RNA in bacteria and yeast using RNA cyclase ribozymes derived from a group I intron of phage T4. Proceedings of the National Academy of Sciences of the United States of America, 1994. 91(8): p. 3117-21.
82. Martinez-Salas, E., et al., Picornavirus IRES elements: RNA structure and host protein interactions. Virus research, 2015.
83. Jackson, R. J., The current status of vertebrate cellular mRNA IRESs. Cold Spring Harbor perspectives in biology, 2013. 5(2).
84. Balvay, L., et al., Structural and functional diversity of viral IRESes. Biochimica et biophysica acta, 2009. 1789(9-10): p. 542-57.
85. Niepmann, M., Internal translation initiation of picornaviruses and hepatitis C virus. Biochimica et biophysica acta, 2009. 1789(9-10): p. 529-41.
86. Romanelli, M. G., E. Diani, and P. M. Lievens, New insights into functional roles of the polypyrimidine tract-binding protein. International journal of molecular sciences, 2013. 14(11): p. 22906-32.
87. Peng, L., X. Q. Yuan, and G. C. Li, The emerging landscape of circular RNA ciRS-7 in cancer (Review). Oncology reports, 2015.
88. Tay, Y., J. Rinn, and P. P. Pandolfi, The multilayered complexity of ceRNA crosstalk and competition. Nature, 2014. 505(7483): p. 344-52.
89. Sen, R., et al., Competing endogenous RNA: the key to posttranscriptional regulation. The Scientific World Journal, 2014. 2014: p. 896206.
90. Bak, R. O. and J. G. Mikkelsen, miRNA sponges: soaking up miRNAs for regulation of gene expression. Wiley interdisciplinary reviews. RNA, 2014. 5(3): p. 317-33.
91. Hansen, T. B., J. Kjems, and C. K. Damgaard, Circular RNA and miR-7 in cancer. Cancer research, 2013. 73(18): p. 5609-12.
92. Bak, R. O., A. K. Hollensen, and J. G. Mikkelsen, Managing microRNAs with vector-encoded decoy-type inhibitors. Molecular therapy: the journal of the American Society of Gene Therapy, 2013. 21(8): p. 1478-85.
93. Schrunder, J., N. Gunge, and F. Meinhardt, Extranuclear expression of the bacterial xylose isomerase (xylA) and the UDP-glucose dehydrogenase (hasB) genes in yeast with Kluyveromyces lactis linear killer plasmids as vectors. Current microbiology, 1996. 33(5): p. 323-30.
94. Schrunder, J. and F. Meinhardt, An extranuclear expression system for analysis of cytoplasmic promoters of yeast linear killer plasmids. Plasmid, 1995. 33(2): p. 139-51.
95. Meinhardt, F., et al., A novel approach to express a heterologous gene on Kluyveromyces lactis linear killer plasmids: expression of the bacterial aph gene from a cytoplasmic promoter fragment without in-phase fusion to the plasmid open reading frame. Plasmid. 1994. 32(3): p. 318-27.
96. Lo, A. S., Q. Zhu, and W. A. Marasco, Intracellular antibodies (intrabodies) and their therapeutic potential. Handbook of experimental pharmacology, 2008(181): p. 343-73.
97. Williams, B. R. and Z. Zhu, Intrabody-based approaches to cancer therapy: status and prospects. Current medicinal chemistry, 2006. 13(12): p. 1473-80.
98. Stocks, M., Intrabodies as drug discovery tools and therapeutics. Current opinion in chemical biology, 2005. 9(4): p. 359-65.
99. Kennedy, E. M. and B. R. Cullen, Bacterial CRISPR/Cas DNA endonucleases: A revolutionary technology that could dramatically impact viral research and treatment. Virology, 2015.
100. Xu, T., et al., Cas9-based tools for targeted genome editing and transcriptional control. Applied and environmental microbiology, 2014. 80(5): p. 1544-52.
101. Wilkinson, R. and B. Wiedenheft, A CRISPR method for genome engineering. F1000prime reports, 2014. 6: p. 3.
102. Terns, R. M. and M. P. Terns, CRISPR-based technologies: prokaryotic defense weapons repurposed. Trends in genetics: TIG, 2014. 30(3): p. 111-8.
103. Sampson, T. R. and D. S. Weiss, Exploiting CRISPR/Cas systems for biotechnology. BioEssays: news and reviews in molecular, cellular and developmental biology, 2014. 36(1): p. 34-8.

TABLE 3
Non-limiting examples of Biologically Active RNA
Sequences

| | | | SEQ |
|---|---|---|---|
| | Name | Nucleotide Sequence | ID NO |
| 1 | p53 | CCAGGGAGCAGGUAGCUGCUGGGCUC | 1 |
| | (mRNA) | CGGGGACACUUUGCGUUCGGGCUGGG | |
| | | AGCGUGCUUUCCACGACGGUGACACG | |
| | | CUUCCCUGGAUUGGCAGCCAGACUGCC | |
| | | UUCCGGGUCACUGCCAUGGAGGAGCC | |
| | | GCAGUCAGAUCCUAGCGUCGAGCCCCC | |
| | | UCUGAGUCAGGAAACAUUUUCAGACC | |
| | | UAUGGAAACUACUUCCUGAAAACAAC | |
| | | GUUCUGUCCCCCUUGCCGUCCCAAGCA | |
| | | AUGGAUGAUUUGAUGCUGUCCCCGGA | |
| | | CGAUAUUGAACAAUGGUUCACUGAAG | |
| | | ACCCAGGUCCAGAUGAAGCUCCCAGA | |
| | | AUGCCAGAGGCUGCUCCCCGCGUGGCC | |
| | | CCUGCACCAGCAGCUCCUACACCGGCG | |
| | | GCCCCUGCACCAGCCCCCUCCUGGCCC | |
| | | CUGUCAUCUUCUGUCCCUUCCCAGAAA | |
| | | ACCUACCAGGGCAGCUACGGUUUCCG | |
| | | UCUGGGCUUCUUGCAUUCUGGGACAG | |
| | | CCAAGUCUGUGACUUGCACGUACUCCC | |
| | | CUGCCCUCAACAAGAUGUUUUGCCAA | |
| | | CUGGCCAAGACCUGCCCUGUGCAGCUG | |
| | | UGGGUUGAUUCCACACCCCCGCCCGGC | |
| | | ACCCGCGUCCGCGCCAUGGCCAUCUAC | |
| | | AAGCAGUCACAGCACAUGACGGAGGU | |
| | | UGUGAGGCGCUGCCCCCACCAUGAGCG | |
| | | CUGCUCAGAUAGCGAUGGUCUGGCCC | |
| | | CUCCUCAGCAUCUUAUCCGAGUGGAA | |
| | | GGAAAUUUGCGUGUGGAGUALTUUGGA | |
| | | UGACAGAAACACUUUUCGACAUAGUG | |
| | | UGGUGGUGCCCUAUGAGCCGCCUGAG | |
| | | GUUGGCUCUGACUGUACCACCAUCCAC | |
| | | UACAACUACAUGUGUAACAGUUCCUG | |
| | | CAUGGGCGGCAUGAACCGGAGGCCCA | |
| | | UCCUCACCAUCAUCACACUGGAAGACU | |
| | | CCAGUGGUAAUCUACUGGGACGGAAC | |
| | | AGCUUUGAGGUGCGUGUUUGUGCCUG | |
| | | UGCUGGGAGAGACCGGCGCACAGAGG | |
| | | AAGAGAAUCUCCGCAAGAAAGGGGAG | |
| | | CCUCACCACGAGCUGCCCCCAGGGAGC | |
| | | ACUAAGCGAGCACUGCCCAACAACACC | |
| | | AGCUCCUCUCCCCAGCCAAAGAAGAAA | |
| | | CCACUGGAUGGAGAAUAUUUCACCCU | |
| | | UCAGAUCCGUGGGCGUGAGCGCUUCG | |
| | | AGAUGUUCCGAGAGCUGAAUGAGGCC | |
| | | UUGGAACUCAAGGAUGCCCAGGCUGG | |
| | | GAAGGAGCCAGGGGGGAGCAGGGCUC | |
| | | ACUCCAGCCACCUGAAGUCCAAAAAG | |
| | | GGUCAGUCUACCUCCCGCCAUAAAAA | |
| | | ACUCAUGUUCAAGACAGAAGGGCCUG | |
| | | ACUCAGACUGACAUUCUCCACUUCUU | |
| | | GUUCCCCACUGACAGCCUCCCACCCCC | |
| | | AUCUCUCCCUCCCCUGCCAUUUUGGGU | |
| | | UUUGGGUCUUUGAACCCUUGCUUGCA | |
| | | AUAGGUGUGCGUCAGAAGCACCCAGG | |
| | | ACUUCCAUUUGCUUUGUCCCGGGGCU | |
| | | CCACUGAACAAGUUGGCCUGCACUGG | |
| | | UGUUUUGUUGUGGGGAGGAGGAUGGG | |
| | | GAGUAGGACAUACCAGCUUAGAUUUU | |
| | | AAGGUUUUUACUGUGAGGGAUGUUUG | |
| | | GGAGAUGUAAGAAAUGUUCUUGCAGU | |
| | | UAAGGGUUAGUUUACAAUCAGCCACA | |
| | | UUCUAGGUAGGGGCCCACUUCACCGU | |
| | | ACUAACCAGGGAAGCUGUCCCUCACU | |
| | | GUUGAAUUUUCUCUAACUUCAAGGCC | |
| | | CAUAUCUGUGAAAUGCUGGCAUUUGC | |
| | | ACCUACCUCACAGAGUGCAUUGUGAG | |
| | | GGUUAAUGAAAUAAUGUACAUCUGGC | |
| | | CUUGAAACCACCUUUUAUUACAUGGG | |
| | | GUCUAGAACUUGACCCCCUUGAGGGU | |
| | | GCUUGUUCCCUCUCCCUGUUGGUCGG | |
| | | UGGGUUGGUAGUUUCUACAGUUGGGC | |
| | | AGCUGGUUAGGUAGAGGGAGUUGUCA | |
| | | AGUCUCUGCUGGCCCAGCCAAACCCUG | |
| | | UCUGACAACCUCUUGGUGAACCUUAG | |
| | | UACCUAAAAGGAAAUCUCACCCCAUCC | |
| | | CACACCCUGGAGGAUUUCAUCUCUUG | |
| | | UAUAUGAUGAUCUGGAUCCACCAAGA | |
| | | CUUGUUUUAUGCUCAGGGUCAAUUUC | |
| | | UUUUUUUUUUUUUUUUUUUUUUUUCU | |
| | | UUUUCUUUGAGACUGGGUCUCGCUU | |
| | | GUUGCCCAGGCUGGAGUGGAGUGGCG | |
| | | UGAUCUUGGCUUACUGCAGCCUUUGC | |
| | | CUCCCCGGCUCGAGCAGUCCUGCCUCA | |
| | | GCCUCCGGAGUAGCUGGGACCACAGG | |
| | | UUCAUGCCACCAUGGCCAGCCAACUUU | |
| | | UGCAUGUUUUGUAGAGAUGGGGUCUC | |
| | | ACAGUGUUGCCCAGGCUGGUCUCAAA | |
| | | CUCCUGGGCUCAGGCGAUCCACCUGUC | |
| | | UCAGCCUCCCAGAGUGCUGGGAUUAC | |
| | | AAUUGUGAGCCACCACGUCCAGCUGG | |
| | | AAGGGUCAACAUCUUUUACAUUCUGC | |
| | | AAGCACAUCUGCAUUUUCACCCCACCC | |
| | | UUCCCCUCCUUCUCCCUUUUUAUAUCC | |
| | | CAUUUUAUAUCGAUCUCUUAUUUUA | |
| | | CAAUAAAACUUUGCUGCCAAAAANAA | |
| | | AAAAAAAAAAAA | |
| 2 | K-Ras G12V | GUUGGAGCUGUUGGCGUAGUUCAAGA | 2 |
| | (shRNA) | GACUACGCCAACAGCUCCAACUUU | |
| 3 | K-Ras G12C | GUUGGAGCUUGUGGCGUAGUUCAAGA | 3 |
| | (shRNA) | GACUACGCCACAAGCUCCAACUUU | |
| 4 | K-Ras G12D | GUUGGAGCUUGUGGCGUAGUUCAAGA | 4 |
| | (shRNA) | GACUACGCCACAAGCUCCAACUUU | |
| 5 | EGFR | CAGAGGAUGUUCAAUAACUUUCAAGA | 5 |
| | (shRNA) | GAAGUUAUUGAACAUCCUCUGUUU | |
| 6 | c-Myc | UGAGACAGAUCAGCAACAAUUCAAGA | 6 |
| | (shRNA) | GAUUGUUGCUGAUCUGUCUCAUUU | |
| 7 | bcl-2 | GGAUGACUGAGUACCUGAACCUCGAG | 7 |
| | (shRNA) | GUUCAGGUACUCAGUCAUCCAUUU | |
| 8 | Survivin | GGCUGGCUUCAUCCACUGCUUCAAGA | 8 |
| | (shRNA) | GAGCAGUGGAUGAAGCCAGCCUUU | |
| 9 | FAK | AACCACCUGGGCCAGUAUUAUCUCGA | 9 |
| | (shRNA) | GAUAAUACUGGCCCAGGUGGUUU | |
| 10 | STAT3 | GAGAUUGACCAGCAGUAUAUUCAAGA | 10 |
| | (shRNA) | GAUAUACUGCUGGUCAAUCUCUUU | |
| 11 | HER3 | CGCGUGUGCCAGCGAAAGUUGCGUAU | 11 |
| | (shRNA) | GGGUCACAUCGCAGGCACAUGUCAUC | |
| | | UGGGCGGUCCGUUCGUUU | |
| 12 | β-catenin | GGACGCGUGGUACCAGGCCGAUCUAU | 12 |
| | (shRNA) | GGACGCUAUAGGCACACCGGAUACUU | |
| | | UAACGAUUGGCUAAGCUUCCGCGGGG | |
| | | AUCUUU | |
| 13 | Src | UCAGAGCGGUUACUGCUCAAUCUCGA | 13 |
| | (shRNA) | GAUUGAGCAGUAACCGCUCUGAUUU | |
| 14 | HSF1 | GCAGGUUGUUCAUAGUCAGAAUUCAA | 14 |
| | (shRNA) | GAGAUUCUGACUAUGAACAACCUGCU | |
| | | UU | |

TABLE 4
Non-limiting examples of Immune Masking Protein sequences

| | | |
|---|---|---|
| Name | Amino Acid Sequence | SEQ ID NO |

| | | | |
|---|---|---|---|
| 1 | RodA | MKFSLSAAVLAFAVSVAALPQHMTNAA | 15 |
| | hydrophobin | GNGVGNKGNANVRFPVPDDITVKQATEK | |
| | (A. Fumigatis) | CGDQAQLSCCNKATYAGDVTDIDEGILA | |
| | | GTLKNLIGGGSGTEGLGLFNQCSNVDLQI | |
| | | PVIGIPIQALVNQKCKQNIACCQNSPSDAS | |
| | | GSLIGLGLPCIALGSIL | |
| 2 | Sjc23-LED | YKDKIDDEINTLMTGALENPNEEITATMC | 16 |
| | tetraspanin | KIQTSFHCCGVKGPDDYKGNVPASCKEG | |
| | (S. japonicum) | QEVYVQGCLSVFSAFLKRN | |
| 3 | Sjc23-Min | KIQTSFHCC | 17 |
| | (S. japonicum) | | |
| 4 | Elastin-Like | VPGSGVPGSGVPGGGVPGSGVPGSGVPG | 18 |
| | Polypeptide | GGVPGSGVPGSG | |
| | ELP_{SG} | | |
| 5 | Elastin-Like | VPGKGVPGKGVPGGGVPGKGVPGKGVP | 19 |
| | Polypeptide | GGGVPGKGVPGKG | |
| | ELP_{AG} | | |

TABLE 5
Non-limiting examples of Targeting Peptide
sequences

| | Name | Amino Acid Sequence | SEQ ID NO |
|---|---|---|---|
| 1 | RGD-4C | CDCRGDCFC | 20 |
| 2 | NGR | CNGRCVSGCAGRC | 21 |
| 3 | LHRH | QHWSYKLRP | 22 |
| 4 | DV3 (CXCR4) | LGASWHRPDKG | 23 |
| 5 | CREKA | CREKA | 24 |
| 6 | PH1 | TMGFTAPRFPHY | 25 |
| 7 | bFGFp | KRTGQYKLC | 26 |
| 8 | GE11 (EGFR) | YHWYGYTPQNVI | 27 |
| 9 | Transferrin | THRPPMWSPVWP | 28 |
| 10 | GFE1 (Lung) | CGFECVRQCPERC | 29 |
| 11 | Anti-Flt1 | GNQWFI | 30 |
| | (VEGFR) | | |

TABLE 6
Non-limiting examples of Yeast Linear Plasmid backbones

| | | | Pubmed |
|---|---|---|---|
| | | | Assession |
| | Name | Yeast parent strain | Numbers |
| 1 | pGKL1 and | Kluyveromyces lactis | X01095 1 |
| | pGKL2 | | |
| 2 | pPin1-1, pPin1-2 | Pichia inositovora | AJ564102 |
| | and pPin1-3 | | |
| 3 | pSKL | Saccharomyces kluyveri | X54850 |
| 4 | pDHL1, | Debaryomyces hansenii | AJ011124 |
| | pDHL2 and | | |
| | pDHL3 | | |
| 5 | pWR1A and | Wingea robertsiae | AJ617332 |
| | pWR1B | | |
| 6 | pPac1-1 and | Pichia acacia | AM180622 |
| | pPac1-2 | | |

TABLE 7
Non-limiting examples of Yeast Promoter Sequences

| | | | SEQ |
|---|---|---|---|
| | | | ID |
| | Name | Nucleotide Sequence | NO |
| 1 | scGPD | CATTATCAATACTCGCCATTTCAAAGAATA | 31 |
| | | CGTAAATAATTAATAGTAGTGATTTTCCTA | |
| | | ACTTTATTTAGTCAAAAAATTAGCCTTTTAA | |
| | | TTCTGCTGTAACCCGTACATGCCCAAAATA | |
| | | GGGGGCGGGTTACACAGAATATATAACATC | |
| | | GTAGGTGTCTGGGTGAACAGTTTATTCCTG | |
| | | GCATCCACTAAATATAATGGAGCCCGCTTT | |
| 2 | scTEF | TTAAGCTGGCATCCAGAAAAAAAAAGAATC | 32 |
| | | CCAGCACCAAAATATTGTTTTCTTCACCAAC | |
| | | CATCAGTTCATAGGTCCATTCTCTTAGCGCA | |
| | | ACTACAGAGAACAGGGGCACAAACAGGCA | |
| | | AAAAACGGGCACAACCTCAATGGAGTGAT | |
| | | GCAACCTGCCTGGAGTAAATGATGACACAA | |
| | | GGCAATTGACCCACGCATGTATCTATCTCA | |
| | | TTTTCTTACACCTTCTATTACCTTCTGCTCTC | |
| | | TCTGATTTGGAAAAAGCTGAAAAAAAAGGT | |
| | | TGAAACCAGTTCCCTGAAATTATTCCCCTAC | |
| | | TTGACTAATAAGTATATAAAGACGGTAGGT | |
| | | ATTGATTGTAATTCTGTAAATCTATTTCTTA | |
| | | AACTTCTTAAATTCTACTTTTATAGTTAGTC | |
| | | TTTTTTTTAGTTTTAAAACACCAGAACTTAG | |
| | | TTTCGA | |
| 3 | scGAL1 | CGGATTAGAAGCCGCCGAGCGGGTGACAG | 33 |
| | | CCCTCCGAAGGAAGACTCTCCTCCGTGCGT | |
| | | CCTCGTCTTCACCGGTCGCGTTCCTGAAACG | |
| | | CAGATGTGCCTCGCGCCGCACTGCTCCGAA | |
| | | CAATAAAGATTCTACAATACTAGCTTTTAT | |
| | | GGTTATGAAGAGGAAAAATTGGCAGTAACC | |
| | | TGGCCCCACAAACCTTCAAATGAACGAATC | |
| | | AAATTAACAACCATAGGATGATAATGCGAT | |
| | | TAGTTTTTTAGCCTTATTTCTGGGGTAATTA | |
| | | ATCAGCGAAGCGATGATTTTTGATCTATTA | |
| | | ACAGATATATAAATGCAAAAACTGCATAAC | |
| | | CACTTTAACTAATACTTTCAACATTTTCGGT | |
| | | TTGTATTACTTCTTATTCAAATGTAATAAAA | |
| | | GTATCAACAAAAAATTGTTAATATACCTCT | |
| | | ATACTTTAACGTCAAGGA | |
| 4 | scADH1 | ATCCTTTTGTTGTTTCCGGGTGTACAATATG | 34 |
| | | GACTTCCTCTTTTCTGGCAACCAAACCCATA | |
| | | CATCGGGATTCCTATAATACCTTCGTTGGTC | |
| | | TCCCTAACATGTAGGTGGCGGAGGGGAGAT | |
| | | ATACAATAGAACAGATACCAGACAAGACA | |
| | | TAATGGGCTAAACAAGACTACACCAATTAC | |
| | | ACTGCCTCATTGATGGTGGTACATAACGAA | |
| | | CTAATACTGTAGCCCTAGACTTGATAGCCA | |
| | | TCATCATATCGAAGTTTCACTACCCTTTTTC | |
| | | CATTTGCCATCTATTGAAGTAATAATAGGC | |
| | | GCATGCAACTTCTTTTCTTTTTTTTTCTTTTC | |
| | | TCTCTCCCCCGTTGTTGTCTCACCATATCCG | |
| | | CAATGACAAAAAAATGATGGAAGACACTA | |
| | | AAGGAAAAAATTAACGACAAAGACAGCAC | |
| | | CAACAGATGTCGTTGTTCCAGAGCTGATGA | |
| | | GGGGTATCTCGAAGCACACGAAACTTTTTC | |
| | | CTTCCTTCATTCACGCACACTACTCTCTAAT | |
| | | GAGCAACGGTATACGGCCTTCCTTCCAGTT | |
| | | ACTTGAATTTGAAATAAAAAAAAGTTTGCT | |
| | | GTCTTGCTATCAAGTATAAATAGACCTGCA | |
| | | ATTATTAATCTTTTGTTTCCTCGTCATTGTTC | |
| | | TCGTTCCCTTTCTTCCTTGTTTCTTTTTCTGC | |
| | | ACAATATTTCAAGCTATACCAAGCATACAA | |
| | | TCAACT | |
| 5 | scMET17, | TTATTTTTTGCTTTTTCTCTTGAGGTCACATG | 35 |
| | 25 | ATCGCAAAATGGCAAATGGCACGTGAAGCT | |
| | | GTCGATATTGGGGAACTGTGGTGGTTGGCA | |
| | | AATGACTAATTAAGTTAGTCAAGGCGCCAT | |
| | | CCTCATGAAAACTGTGTAACATAATAACCG | |
| | | AAGTGTCGAAAAGGTGGCACCTTGTCCAAT | |
| | | TGAACACGCTCGATGAAAAAAATAAGATAT | |
| | | ATATAAGGTTAAGTAAAGCGTCTGTTAGAA | |
| | | AGGAAGTTTTTCCTTTTTCTTGCTCTCTTGTC | |
| | | TTTTCATCTACTATTTCCTTCGTGTAATACA | |
| | | GGGTCGTCAGATACATAGATACAATTCTAT | |
| | | TACCCCCATCCATAC | |
| 6 | ppAOX1 | AACATCCAAAGACGAAAGGTTGAATGAAA | 36 |
| | | CCTTTTTGCCATCCGACATCCACAGGTCCAT | |
| | | TCTCACACATAAGTGCCAAACGCAACAGGA | |
| | | GGGGATACACTAGCAGCAGACCGTTGCAAA | |
| | | CGCAGGACCTCCACTCCTCTTCTCCTCAACA | |
| | | CCCACTTTTGCCATCGAAAAACCAGCCCAG | |
| | | TTATTGGGCTTGATTGGAGCTCGCTCATTCC | |
| | | AATTCCTTCTATTAGGCTACTAACACCATGA | |
| | | CTTTATTAGCCTGTCTATCCTGGCCCCCCTG | |
| | | GCGAGGTTCATGTTTGTTTATTTCCGAATGC | |
| | | AACAAGCTCCGCATTACACCCGAACATCAC | |
| | | TCCAGATGAGGGCTTTCTGAGTGTGGGGTC | |
| | | AAATAGTTTCATGTTCCCCAAATGGCCCAA | |
| | | AACTGACAGTTTAAACGCTGTCTTGGAACC | |
| | | TAATATGACAAAAGCGTGATCTCATCCAAG | |
| | | ATGAACTAAGTTTGGTTCGTTGAAATGCTA | |
| | | ACGGCCAGTTGGTCAAAAAGAAACTTCCAA | |
| | | AAGTCGGCATACCGTTTGTCTTGTTTGGTAT | |
| | | TGATTGACGAATGCTCAAAAATAATCTCAT | |
| | | TAATGCTTAGCGCAGTCTCTCTATCGCTTCT | |
| | | GAACCCCGGTGCACCTGTGCCGAAACGCAA | |
| | | ATGGGGAAACACCCGCTTTTTGGATGATTA | |
| | | TGCATTGTCTCCACATTGTATGCTTCCAAGA | |
| | | TTCTGGTGGGAATACTGCTGATAGCCTAAC | |
| | | GTTCATGATCAAAATTTAACTGTTCTAACCC | |
| | | CTACTTGACAGCAATATATAAACAGAAGGA | |
| | | AGCTGCCCTGTCTTAAACCTTTTTTTTTATC | |
| | | ATCATTATTAGCTTACTTTCATAATTGCGAC | |
| | | TGGTTCCAATTGACAAGCTTTTGATTTTAAC | |
| | | GACTTTTAACGACAACTTGAGAAGATCAAA | |
| | | AAACAACTAATTATTGAAA | |
| 7 | cgEGD2 | TGTCCACTTCACTCACCAGTAATAAGTTGCC | 37 |
| | | CAGTCACCATCCCTTATATACGCTGTGGCTA | |
| | | CCGAGATGGCCGAACCCACTCCAAATATAT | |
| | | GGGATACTCAAACTGAAAGATGATGATGAG | |
| | | CTTATTATGGTAATGCTGTACGTACATTACC | |
| | | CCCGTGGCAAGAAACACGTACAACGCTACA | |
| | | TTTCTCGTCGACGCTTTTTTTTTTCAAAATTT | |
| | | CTTTCATCGAAGAGCTTTGGAGGGTCTCAT | |
| | | AGCACATATAAGTTGTTATTGCTACACAAT | |
| | | TTTGGTCAATTGAGTAGCTGTTGCAATAGG | |
| | | GACAAATCAAAAACACGTCAATAATACAG | |
| | | ATATACAAAG | |
| 8 | cgPDC2 | AGCATTTTTATACACGTTTTACGTATTTTTT | 38 |
| | | TTGCAATACACATAGATACGTACGTACAAC | |
| | | CCTTTCTATTGTGAGAACTTTGTAACACTCT | |
| | | TTTGTAGACCAGTGTAGTCAGCAAGCATCA | |
| | | GCAAGCATCAGCAAGCATCAGCAAGCATCA | |
| | | GCTAGTCTAAAGCCTTTAGCAAAGTCAAAA | |
| | | ACCAACACCCACACACCGGGATCCCACCTT | |
| | | CCCCGAGATGCCTGTTCCCGACTCCCAGCT | |
| | | GCTCCCACGACGGGGAGGAATCAGAAGAC | |
| | | GGAGATAGAAAGAGAGCGTTGAACGCGCG | |
| | | CGATGCCAATGAGAGATAAAAAAAAAGAA | |
| | | ATACCAATCCCACGACATATTCTAACGGGC | |
| | | TCCACAGACAATGCACAACAATCGAGATGG | |
| | | GATTGAATCACAGAGAGAGAGATCAGAAA | |
| | | AAAAAGGAACTGCAAGGGTCCCCACCCCA | |
| | | AACTCCCCTCCGCTATCCACTACAATCGCC | |
| | | GCACCGACAAAATCCATTGTGTCCGCAACG | |
| | | ACCAGAGCAAAGAGCCAAAAACACAATTG | |
| | | AATAACAAAGAAAAGGGCAGAGAGCCACT | |
| | | TAGACAATTACGTAGGAACCATATTTTTGC | |
| | | ATAACTTTATGCAATTTGCCCAAATTCAAGT | |
| | | GAAGCGAGAGAAGCCAGAGAGCTACAGCT | |
| | | GAAGAGCTACAGCTGAAGTGAAAAGCCAA | |
| | | GAATCTCTTAGAATATACCGTTTGGCTTATT | |
| | | TGAATCAAATATTTATGCGCTTAAATCCCAT | |
| | | AAAAAAGCAATTTATACAGATAAGTCTGCA | |
| | | GAAAGAAAAATTTTCTTCTTGATACCTGAA | |
| | | CAAAGAAATCAAACTCATCAAGAATAAATC | |
| | | AATTCATGAAAAAAAAACATATATAAAGG | |
| | | ACAACATGGAATCAAGTTTCAATAATTTTT | |
| | | AGATTGTACATAAATAAAGAGACCAGACTA | |
| | | ATACAACTGTATAAGCTCTAAACATTCAAT | |
| | | TGCCAAAAAACATTAACA | |
| 9 | cgHHT2 | TGTTATTGATTATTTATTTATTTGTTGTTATT | |
| | | TTATTGTATTTTTTTTTAAAAAGCTACTAAT | |
| | | ACACAACAAAATGTAAACCACAAACTCTCC | |
| | | CAAACAGAACGAACACCCTGGCTTTATATA | |
| | | CAAAATTTCTTATCCCGAAATGATTGTCAA | |
| | | CAAGATAAACAAAAAGAAGCCACCACAGG | |
| | | AACCCTACCCCCCCTCTCTCCCACCCCCATC | |
| | | GTGTCTTCCCTTCCACCTTCCACCCTTCACC | |
| | | TGGCCATAGTAACTTGAGGTGTGCACACTC | |
| | | CAGAACGGTCGACCCACTACGGATCTGAAT | |
| | | TCCAGTGCGAAGTTGCGGGAACGGATTTAT | |
| | | TATGACCAGGCGGCTACTAAGAAGCTCAAA | |
| | | CCATCAAAAACGCGCGAAGAAACTGGTCTA | |
| | | CCCGTAGCAAGTTACTATTCTCCAAATTAGT | |
| | | GTTTGTCGTGTGCCGTGCGTCGTGGGTAGA | |
| | | CCGAGATTTTCGCGCGCCTCAGGTGGTTCG | |
| | | AGGGCAAATAACCATAGTTAAGCCGTTCCC | |
| | | ATTCTTTCTCACATGTACGTGCCGGACGCTT | |
| | | CGATGAAATTTTTGTGAATCGTTTCGTTGCT | |
| | | TCTGTGACCACAAAACAGCCTGGAGAGGAG | |
| | | GCGTTGCGTAAGGGGGGGGGGCCTGATGA | |
| | | GAAACAGATGACACAACGCAGAACTGGCT | |
| | | GCAGTGCAAAGGCATTTCCTCTGCCACACA | |
| | | CACTACCATAACCAGTCTCGGTATTAACTGG | |
| | | CTCAACAAACGATAAGAGCCAAAGAGGAA | |
| | | GAAAAAAAGGTATTTAAAGGATGTATTTCT | |
| | | TTATCTCCTGATTAGATTTCTTACTTGTACA | |
| | | GAAACTGTAACTAAAAAAAACAACAACAA | |
| | | AACACAACACATACATA | |
| 10 | cgMET3 | CAAGTACAAATTATGTAATATGGTTATAAT | 40 |
| | | TGGCTTATATGTATGGAGGTAGGGAACTGC | |
| | | AGGTTATTGTTCTGTTAAACTACCAGTAATG | |
| | | CAAGACGGCTAAGTCATATGACTGCCACTT | |
| | | TGTCAATCCTGAAGAAAATGACAATAGAAA | |
| | | GTGATATAATACACGTGATATGCGTAGGAT | |
| | | GGGACCCAAAGATAGCCACATCACATGATC | |
| | | TTCAAAAACCCCGTCAAAAAGGAATAAATC | |
| | | GAAGGAAAAAAAATGCCACGTGACTTTGAT | |
| | | AGTCAAAATCAGGTTATATTACTATACGAT | |
| | | CCCCCACACATAACCTTTACCACACACCGC | |
| | | ACGGGCTGGACTTATCCTATTAGAAAGCGG | |
| | | TGCAGCCAGGGCCAAGA4AACGCGAACGA | |
| | | CGCAGAAAAAACGTCAAGCAAAAAAACTG | |
| | | TGGTGTTATTTAAAGCATACAATTTCCTGCC | |
| | | TCCCTTTAATGCTACGGGGGATCTAGCAAA | |
| | | TGGGAAAATATCACATGACTTTGGCTAGAA | |
| | | GGGGTGGGAAAACAGATATTTTAGTCACAT | |
| | | TGTTTGATTTCACGTACTACACGATACACTA | |
| | | CACTACACAATACACTGCAATACACTACAC | |
| | | TGTATGGTTCCCTCCGGCCCTGGATATGCTA | |
| | | GCGAAGGATCCCAAGCCCATCGAGGAAATC | |
| | | ATTCAAGAGCATCTGCAGGTCTCAAATACA | |
| | | CTAAGTCCAAACACAAACAGCACTGAGCTA | |
| | | ATCCAAGACACCAATTGCATGCCTTCTCAA | |
| | | TTAGAACAGTTTATTACACAGCTTATAACT | |
| | | GTGCATCTTGCCATTCTTTTCGAGATAGCCA | |
| | | TTTGCATTTGATTAACATGCTTAGCTCGTTC | |
| | | TCAAGCCACAGTAAAATGGATTGCCTTTTG | |
| | | AGTTTCCATCGTTGTATAAATAGGCACTCAT | |
| | | TCCTATAGCCTTGCTCTTTGGTCTTGTCTCT | |
| | | GTAATGGTAATAGCTGTAAGTCAGGATATA | |
| | | ACACTCCAGAAAAGAAACACCTAACAATCT | |
| | | AGA | |

TABLE 8
Non-limiting examples of Yeast Terminator
sequences

| | | | SEQ |
|---|---|---|---|
| | Name | Nucleotide Sequence | ID NO |
| 1 | CYC1 | TCATGTAATTAGTTATGTCACGCTTACATTC | 41 |
| | | ACTGTSDGCCCTCCCCCCACATCCGCTCTAA | |
| | | CCGAAAAGGAAGGAGTTAGACAACCTGAA | |
| | | GTCTAGGTCCCTATTTATTTTTTTATAGTTA | |
| | | TGTTAGTATTAAGAACGTTATTTATATTTCA | |
| | | AATTTTTCTTTTTTTTCTGTACAGACGCGTG | |
| | | TACGCATGTAACATTATACTGAAAACCTTG | |
| | | CTTGAGAAGGTTTTGGGACGCTCGAAGGCT | |
| | | TTAATTTGC | |
| 3 | ADH1 | GCGAATTTCTTATGATTTATGATTTTTATTA | 42 |
| | | TTAAATAAGTTATAAAAAAAATAAGTGTAT | |
| | | ACAAATTTTAAAGTGAGTCTTAGGTTTTAA | |
| | | AACGAAAATTCTTATTCTTGAGTAACTCTTT | |
| | | CCTGTAGGTCAGGTTGCTTTCTCAGGTATAG | |
| | | TATGAGGTCGCTCTTATTGACCACACCTCTA | |
| | | C | |

TABLE 9
Non-limiting examples of Mammalian Promoter sequences

| | | Nucleotide | |
|---|---|---|---|
| | Name | Sequence | SEQ ID NO |
| 1 | SV40 | GCGCAGCACCATGGCCTGAAATAACCTCTG | 43 |
| | | AAAGAGGAACTTGGTTAGGTACCTTCTGAG | |
| | | GCGGAAAGAACCAGCTGTGGAATGTGTGTC | |
| | | AGTTAGGGTGTGGAAAGTCCCCAGGCTCCC | |
| | | CAGCAGGCAGAAGTATGCAAAGCATGCATC | |
| | | TCAATTAGTCAGCAACCAGGTGTGGAAAGT | |
| | | CCCCAGGCTCCCCAGCAGGCAGAAGTATGC | |
| | | AAAGCATGCATCTCAATTAGTCAGCAACCA | |
| | | TAGTCCCGCCCCTAACTCCGCCCATCCCGCC | |
| | | CCTAACTCCGCCCAGTTCCGCCCATTCTCCG | |
| | | CCCCATGGCTGACTAATTTTTTTTATTTATG | |
| | | CAGAGGCCGAGGCCGCCTCGGCCTCTGAGC | |
| | | TATTCCAGAAGTAGTGAGGAGGCTTTTTTG | |
| | | GAGGCCTAGGCTTTTGCAAAAAGCTT | |
| 2 | Chicken | AGTCGAGGTGAGCCCCACGTTCTGCTTCAC | 44 |
| | β-actin | TCTCCCCATCTCCCCCCCCTCCCCACCCCCA | |
| | | ATTTTGTATTTATTTATTTTTTAATTATTTTG | |
| | | TGCAGCGATGGGGGCGGGGGGGGGGGGGG | |
| | | CGCGCGCCAGGCGGGGCGGGGCGGGGCGA | |
| | | GGGGCGGGGCGGGGCGAGGCGGAGAGGTG | |
| | | CGGCGGCAGCCAATCAGAGCGGCGCGCTCC | |
| | | GAAAGTTTCCTTTTATGGCGAGGCGGCGGC | |
| | | GGCGGCGGCCCTATAAAAAGCGAAGCGCG | |
| | | CGGCGGGCGGGAGTCGCTGCGTTGCCTTCG | |
| | | CCCCGTGCCCCGCTCCGCGCCGCCTCGCGC | |
| | | CGCCCGCCCCGGCTCTGACTGACCGCGTTA | |
| | | CTCCCACAGGTGAGCGGGCGGGACGGCCCT | |
| | | TCTCCTCCGGGCTGTAATTAGCGCTTGGTTT | |
| | | AATGACGGCTCGTTTCTTTTCTGTGGCTGCG | |
| | | TGAAAGCCTTAAAGGGCTCCGGGAGGGCCC | |
| | | TTTGTGCGGGGGGGAGCGGCTCGGGGGGTG | |
| | | CGTGCGTGTGTGTGTGCGTGGGGAGCGCCG | |
| | | CGTGCCTGCCCGCGCTGCCCGGCGGCTGTGA | |
| | | GCGCTGCGGGCGCGGCGCGGGGCTTTGTGC | |
| | | GCTCCGCGTGTGCGCGAGGGGAGCGCGGCC | |
| | | GGGGGCGGTGCCCCGCGGTGCGGGGGGGC | |
| | | TGCGAGGGGAACAAAGGCTGCGTGCGGGG | |
| | | TGTGTGCGTGGGGGGGTGAGCAGGGGGTGT | |
| | | GGGCGCGGCGGTCGGGCTGTAACCCCCCCC | |
| | | TGCACCCCCCTCCCCGAGTTGCTGAGCACG | |
| | | GCCCGGCTTCGGGTGCGGGGCTCCGTGCGG | |
| | | GGCGTGGCGCGGGGCTCGCCGTGCCGGGCG | |
| | | GGGGGTGGCGGCAGGTGGGGGTGCCGGGC | |
| | | GGGGCGGGGCCGCCTCGGGCCGGGGAGGG | |
| | | CTCGGGGGAGGGGCGCGGCGGCCCCGGAG | |
| | | CGCCGGCGGCTGTCGAGGCGCGGCGAGCCG | |
| | | CAGCCATTGCCTTTTATGGTAATCGTGCGA | |
| | | GAGGGCGCAGGGACTTCCTTTGTCCCAAAT | |
| | | CTGGCGGAGCCGAAATCTGGGAGGCGCCGC | |
| | | CGCACCCCCTCTAGCGGGCGCGGGCGAAGC | |
| | | GGTGCGGCGCCGGCAGGAAGGAAATGGGC | |
| | | GGGGAGGGCCTTCGTGCGTCGCCGCGCCGC | |
| | | CGTCCCCTTCTCCATCTCCAGCCTCGGGGCT | |
| | | GCCGCAGGGGGACGGCTGCCTTCGGGGGG | |
| | | GACGGGGCAGGGCGGGGTTCGGCTTCTGGC | |
| | | GTGTGACCGGCGGGGTTTATATCTTCCCTTC | |
| | | TCTGTTCCTCCGCAGCCCCCAA | |
| 3 | CMV | GATCTTCAATATTGGCCATTAGCCATATTAT | 45 |
| | | TCATTGGTTATATAGCATAATCAATATTG | |
| | | GCTATTGGCCATTGCATACGTTGTATCTATA | |
| | | TCATAATATGTACATTTATATTGGCTCATGT | |
| | | CCAATATGACCGCCATGTTGGCATTGATTA | |
| | | TTGACTAGTTATTATTAGTAATCAATTACG | |
| | | GGGTCATTAGTTCATAGCCCATATATGGAG | |
| | | TTCCGCGTTACATAACTTACGGTAAATGGC | |
| | | CCGCCTGGCTGACCGCCCAACGACCCCCGC | |
| | | CCATTGACGTCAATAATGACGTATGTTCCC | |
| | | ATAGTAACGCCAATAGGGACTTTCCATTGA | |
| | | CGTCAATGGGTGGAGTATTTACGGTAAACT | |
| | | GCCCACTTGGCAGTACATCAAGTGTATCAT | |
| | | ATGCCAAGTCCGCCCCCTATTGACGTCAAT | |
| | | GACGGTAAATGGCCCGCCTGGCATTATGCC | |
| | | CAGTACATGACCTTACGGGACTTTCCTACTT | |
| | | GGCAGTACATCTACGTATTAGTCATCGCTA | |
| | | TTACCATGGTGATGCGGTTTTGGCAGTACA | |
| | | CCAATGGGCGTGGATAGCGGTTTGACTCAC | |
| | | GGGGATTTCCAAGTCTCCACCCCATTGACG | |
| | | TCAATGGGAGTTTGTTTTGGCACCAAAATC | |
| | | AACGGGACTTTCCAAAATGTCGTAATAACC | |
| | | CCGCCCCGTTGACGCAAATGGGCGGTAGGC | |
| | | GTGTACGGTGGGAGGTCTATATAAGCAGAG | |
| | | CTCGTTTAGTGAACCGTCA | |
| 4 | Human U6 | AGGGCCTATTTCCCATGATTCCTTCATATTT | 46 |
| | | GCATATACGATACAAGGCTGTTAGAGAGAT | |
| | | AATTGGAATTAATTTGACTGTAAACACAAA | |
| | | GATATTAGTACAAAATACGTGACGTAGAAA | |
| | | GTAATAATTTCTTGGGTAGTTTGCAGTTTTA | |
| | | AAATTATGTTTTAAAATGGACTATCATATG | |
| | | CTTACCGTAACTTGAAAGTATTTCGATTTCT | |
| | | TGGCTTTATATATCTTGTGGAAAGGACGA | |

TABLE 10
Non-limiting examples of Mammalian polyA signal sequences

| | Name | Nucleotide Sequence | SEQ. ID NO |
|---|---|---|---|
| 1 | Bovine Growth | AATAAAATGAGGAAATTGCATCGCATTGTC | 47 |
| | Hormone | TGAGTAGGTGTCATTCTATTCTGGGGGGTG | |
| | | GGGTGGGG | |
| 2 | Human Growth | CTCCCCAGTGCCTCTCCTGGCCCTGGAAGTT | 48 |
| | Hormone | GCCACTCGAGTGCCCACCAGCCTTGTCCTA | |
| | | ATAAAATTAAGTTGCATCATTTTGTCTGACT | |
| | | AGGTGTCCTTCTATAATATTATGGGGTGGA | |
| | | GGGGGGTGGTATGGAGCAAGGGGCAAGTT | |
| | | GGGAAGACAACCTGTAGGGCTGGAGGGGA | |
| | | CCGGTGATGAGGGGAT | |
| 3 | SV40 | ATCTAGATAACTGATCATAATCAGCCATAC | 49 |
| | | CACATTTGTAGAGGTTTTACTTGCTTTAAAA | |
| | | AACCTCCCACACCTCCCCCTGAACCTGAAA | |
| | | CATAAAATGAATGCAATTGTTGTTGTTAAC | |
| | | TTGTTTATTGCAGCTTATAATGGTTACAAAT | |
| | | AAAGCAATAGCATCACAAATTTCACAAATA | |
| | | AAGCATTTTTTTCACTGCATTCTAGTTGTGG | |
| | | TTTGTCCAAACTCATCAATGTATCTTA | |
| 4 | Rabbit Beta- | ACCCCTTCACTGTAGGACAGAGCTTCTAGC | 50 |
| | Globin | AAGAAGCTTTATCCCTCAAATAATAATGAA | |
| | | AATAATAAAACTACTCTAAGAAATTATTTG | |
| | | TGATGGTATTGAGTTTATTTTCCTTGTACTT | |
| | | TTAAATATATGGTCCTCAAGGGA | |
| 5 | Synthetic | AATAAAATATCTTTATTTTCATTACATCTGT | 51 |
| | | GTGTTGGTTTTTTGTGTG | |

1. An extracellular vesicle comprising a vesicle membrane and a biologically active molecule, wherein:
(i) the vesicle is derived from a yeast cell transformed with a polynucleotide which expresses or encodes the biologically active molecule; or
(ii) the vesicle is derived from a yeast cell and the biologically active molecule is produced by the yeast cell.

2. The extracellular vesicle of claim 1, wherein the biologically active molecule does not comprise a secretory domain or secretory domain sequence.

3. The extracellular vesicle of claim 1, wherein polynucleotide is a circular or linear DNA.

4. The extracellular vesicle of claim 1, wherein the vesicle membrane further comprises a targeting peptide.

5. The extracellular vesicle of claim 4, wherein the targeting peptide is selected from a one or more targeting peptides listed in Table 5.

6. The extracellular vesicle of claim 1, wherein the vesicle membrane further comprises an immune masking protein.

7. The extracellular vesicle of claim 6, wherein the immune masking protein is selected from a one or more immune masking proteins listed in Table 4.

8. The extracellular vesicle of claim 1, wherein the vesicle membrane further comprises a CRISPR Cas9 protein and a crRNA guide sequence.

9-10. (canceled)

11. The extracellular vesicle of claim 1, wherein the biologically active molecule is a DNA, a RNA, a peptide, or a protein.

12. The extracellular vesicle of claim 11, wherein the RNA is mRNA, siRNA, RNAi, shRNA, miRNA, RNA ribozyme or RNA aptamer.

13. (canceled)

14. The extracellular vesicle of claim 1, wherein the yeast cell is a non-pathogenic yeast strain or a commensal yeast strain.

15. (canceled)

16. The extracellular vesicle of claim 1, wherein the yeast strain is selected from the group consisting of: Candida glabrata, Saccharomyces cerevisiae, Pichia pastoris, and Kluyveromyces lactis.

17-19. (canceled)

20. The extracellular vesicle of claim 1, wherein the cell wall biosynthesis enzyme chitin synthase 3 has been mutated or deleted from the yeast cell.

21. (canceled)

22. The extracellular vesicle of claim 1, wherein the vesicle is derived from a yeast cell and the biologically active molecule is produced by the yeast cell and does not comprise a secretory domain or secretory domain sequence, and wherein the vesicle membrane comprises a transmembrane protein that functions as a targeting ligand for delivering the biologically active molecule to a mammalian target cell through interaction with a target cell receptor.

23. A method for producing the extracellular vesicle of claim 1 comprising transforming the yeast cells with an expression vector comprising a first polynucleotide and a second polynucleotide, wherein the first polynucleotide encodes a yeast origin of replication and the second polynucleotide encodes a transmembrane targeting ligand.

24. A yeast cell comprising the extracellular vesicle of claim 1.

25. A yeast autonomous cytoplasmic linear expression vector comprising a first polynucleotide, a second polynucleotide, a third polynucleotide and a fourth polynucleotide, wherein the first polynucleotide encodes for a yeast origin of replication, the second polynucleotide encodes for an auxotrophic selectable marker, the third polynucleotide encodes for a mammalian nuclear localization signal, and the fourth polynucleotide encodes for a therapeutic RNA or a therapeutic polypeptide.

26-28. (canceled)

29. A method for purifying extracellular vesicles comprising a biologically active molecule comprising:
a. transforming a yeast cell with an expression vector which expresses or encodes a biologically active molecule and does not comprise a secretory domain sequence,
b. culturing the yeast cell in a growth media under conditions where the vesicles are released into the extracellular growth media,
c. removing the yeast cells from the growth media, and
d. purifying the vesicles from the growth media.

30-32. (canceled)

33. The method of claim 29, wherein the biologically active molecule is a therapeutic yeast autonomous cytoplasmic linear plasmid comprising a first polynucleotide, a second polynucleotide, and a third polynucleotide, wherein the first polynucleotide encodes for a yeast origin of replication, the second polynucleotide encodes for a mammalian nuclear localization signal, and the third polynucleotide encodes a therapeutic polypeptide, the expression of which is driven by a mammalian promoter.

34. The method of claim 29, wherein the biologically active molecule is a therapeutic RNA transcribed from the expression vector comprising a first polynucleotide and a second polynucleotide, wherein the first polynucleotide encodes for a yeast origin of replication and the second polynucleotide encodes a biologically active RNA sequence, the expression of which is driven by a yeast promoter.

35. The method of claim 29, wherein the biologically active molecule is a therapeutic polypeptide encoded by the expression vector comprising a first polynucleotide and a second polynucleotide, wherein the first polynucleotide encodes for a yeast origin of replication and the second polynucleotide encodes an mRNA sequence encoding a therapeutic polypeptide, consisting of a vesicle targeting polypeptide domain and a therapeutic polypeptide domain, with the mRNA expression driven by a yeast promoter.

36. A method for delivering a yeast derived extracellular vesicle comprising a biologically active molecule to mammalian target cells in vitro or in vivo, the method comprising:
(i) adding the vesicles to the growth media of the target cells in vitro under conditions where the vesicles can be taken up through fusion with the cell membrane or endocytosis, resulting in transfer of the biologically active molecule to the target cell; or
(ii) administering the vesicles to a subject under conditions where the vesicles can be taken up by target cells in vivo through fusion with the cell membrane or endocytosis, resulting in transfer of the biologically active molecule to the target cell.

37. (canceled)

38. The method of claim 36, wherein the vesicles are administered to the subject by local or systemic injection.


SEQUENCE LISTING (51 sequences)

SEQ ID NO: 1 - RNA, 2508 nt, homo sapiens [sequence omitted]

SEQ ID NO: 2 - RNA, 50 nt, artificial sequence
  K-Ras G12V shRNA
guuggagcuguuggcguaguucaagagacuacgccaacagcuccaacuuu

SEQ ID NO: 3 - RNA, 50 nt, artificial sequence
  K-Ras G12C shRNA
guuggagcuuguggcguaguucaagagacuacgccacaagcuccaacuuu

SEQ ID NO: 4 - RNA, 50 nt, artificial sequence; identical to SEQ ID NO: 3
  K-Ras G12D shRNA

SEQ ID NO: 5 - RNA, 50 nt, artificial sequence
  EGFR shRNA
cagaggauguucaauaacuuucaagagaaguuauugaacauccucuguuu

SEQ ID NO: 6 - RNA, 50 nt, artificial sequence
  c-Myc shRNA
ugagacagaucagcaacaauucaagagauuguugcugaucugucucauuu

SEQ ID NO: 7 - RNA, 50 nt, artificial sequence
  Bcl-2 shRNA
ggaugacugaguaccugaaccucgagguucagguacucagucauccauuu

SEQ ID NO: 8 - RNA, 50 nt, artificial sequence
  Survivin shRNA
ggcuggcuucauccacugcuucaagagagcaguggaugaagccagccuuu

SEQ ID NO: 9 - RNA, 49 nt, artificial sequence
  FAK shRNA
aaccaccugggccaguauuaucucgagauaauacuggcccaggugguuu

SEQ ID NO: 10 - RNA, 50 nt, artificial sequence
  STAT3 shRNA
gagauugaccagcaguauauucaagagauauacugcuggucaaucucuuu

SEQ ID NO: 11 - RNA, 70 nt, artificial sequence
  HER3 shRNA
cgcgugugccagcgaaaguugcguaugggucacaucgcaggcacaugucaucugggcgguccguucguuu

SEQ ID NO: 12 - RNA, 84 nt, artificial sequence
  beta-catenin shRNA
ggacgcgugguaccaggccgaucuauggacgcuauaggcacaccggauacuuuaacgauuggcuaagcuuccgcggggaucuuu

SEQ ID NO: 13 - RNA, 51 nt, artificial sequence
  Src shRNA
ucagagcgguuacugcucaaucucgagauugagcaguaaccgcucugauuu

SEQ ID NO: 14 - RNA, 54 nt, artificial sequence
  HSF1 shRNA
gcagguuguucauagucagaauucaagagauucugacuaugaacaaccugcuuu

SEQ ID NO: 15 - protein, 159 aa, artificial sequence
  RodA hydrophobin
MKFSLSAAVLAFAVSVAALPQHDVNAAGNGVGNKGNANVRFPVPDDITVKQATEKCGDQAQLSCCNKATYAGDVTDIDEGILAGTLKNLIGGGSGTEGLGLFNQCSNVDLQIPVIGIPIQALVNQKCKQNIACCQNSPSDASGSLIGLGLPCIALGSIL

SEQ ID NO: 16 - protein, 76 aa, artificial sequence
  Sjc23-LED tetraspanin
YKDKIDDEINTLMTGALENPNEEITATMCKIQTSFHCCGVKGPDDYKGNVPASCKEGQEVYVQGCLSVFSAFLKRN

SEQ ID NO: 17 - protein, 9 aa, artificial sequence
  Sjc23-Min
KIQTSFHCC

SEQ ID NO: 18 - protein, 40 aa, artificial sequence
  Elastin-Like Polypeptide ELP SG
VPGSGVPGSGVPGGGVPGSGVPGSGVPGGGVPGSGVPGSG

SEQ ID NO: 19 - protein, 40 aa, artificial sequence
  Elastin-Like Polypeptide ELP AG
VPGKGVPGKGVPGGGVPGKGVPGKGVPGGGVPGKGVPGKG

SEQ ID NO: 20 - protein, 9 aa, artificial sequence
  RGD-4C Targeting Peptide
CDCRGDCFC

SEQ ID NO: 21 - protein, 13 aa, artificial sequence
  NGR Targeting Peptide
CNGRCVSGCAGRC

SEQ ID NO: 22 - protein, 9 aa, artificial sequence
  LHRH Targeting Peptide
QHWSYKLRP

SEQ ID NO: 23 - protein, 11 aa, artificial sequence
  DV3 (CXCR4) Targeting Peptide
LGASWHRPDKG

SEQ ID NO: 24 - protein, 5 aa, artificial sequence
  CREKA Targeting Peptide
CREKA

SEQ ID NO: 25 - protein, 12 aa, artificial sequence
  PH1 Targeting Peptide
TMGFTAPRFPHY

SEQ ID NO: 26 - protein, 9 aa, artificial sequence
  bFGFp Targeting Peptide
KRTGQYKLC

SEQ ID NO: 27 - protein, 12 aa, artificial sequence
  GE11 (EGFR) Targeting Peptide
YHWYGYTPQNVI

SEQ ID NO: 28 - protein, 12 aa, artificial sequence
  Transferrin Targeting Peptide
THRPPMWSPVWP

SEQ ID NO: 29 - protein, 13 aa, artificial sequence
  GFE1 (Lung) Targeting Peptide
CGFECVRQCPERC

SEQ ID NO: 30 - protein, 6 aa, artificial sequence
  Anti-Flt1 (VEGFR) Targeting Peptide
GNQWFI

SEQ ID NO: 31 - DNA, 643 bp, artificial sequence
  scGPD promoter
cattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcga

SEQ ID NO: 32 - DNA, 402 bp, artificial sequence
  scTEF promoter
ttacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaac

SEQ ID NO: 33 - DNA, 441 bp, artificial sequence
  scGAL1 promoter
cggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaagga

SEQ ID NO: 34 - DNA, 705 bp, artificial sequence
  scADH1 promoter
atccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaact

SEQ ID NO: 35 - DNA, 350 bp, artificial sequence
  scMET17,25 promoter
ttattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatac

SEQ ID NO: 36 - DNA, 931 bp, artificial sequence
  ppAOX1 promoter
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaa

SEQ ID NO: 37 - DNA, 344 bp, artificial sequence
  cgEGD2 promoter
tgtccacttcactcaccagtaataagttgcccagtcaccatcccttatatacgctgtggctaccgagatggccgaacccactccaaatatatgggatactcaaactgaaagatgatgatgagcttattatggtaatgctgtacgtacattacccccgtggcaagaaacacgtacaacgctacatttctcgtcgacgcttttttttttcaaaatttctttcatcgaagagctttggagggtctcatagcacatataagttgttattgctacacaattttggtcaattgagtagctgttgcaatagggacaaatcaaaaacacgtcaataatacagatatacaaag

SEQ ID NO: 38 - DNA, 973 bp, artificial sequence
  cgPDC2 promoter
agcatttttatacacgttttacgtattttttttgcaatacacatagatacgtacgtacaaccctttctattgtgagaactttgtaacactcttttgtagaccagtgtagtcagcaagcatcagcaagcatcagcaagcatcagcaagcatcagctagtctaaagcctttagcaaagtcaaaaaccaacacccacacaccgggatcccaccttccccgagatgcctgttcccgactcccagctgctcccacgacggggaggaatcagaagacggagatagaaagagagcgttgaacgcgcgcgatgccaatgagagataaaaaaaaagaaataccaatcccacgacatattctaacgggctccacagacaatgcacaacaatcgagatgggattgaatcacagagagagagatcagaaaaaaaaggaactgcaagggtccccaccccaaactcccctccgctatccactacaatcgccgcaccgacaaaatccattgtgtccgcaacgaccagagcaaagagccaaaaacacaattgaataacaaagaaaagggcagagagccacttagacaattacgtaggaaccatatttttgcataactttatgcaatttgcccaaattcaagtgaagcgagagaagccagagagctacagctgaagagctacagctgaagtgaaaagccaagaatctcttagaatataccgtttggcttatttgaatcaaatatttatgcgcttaaatcccataaaaaagcaatttatacagataagtctgcagaaagaaaaattttcttcttgatacctgaacaaagaaatcaaactcatcaagaataaatcaattcatgaaaaaaaaacatatataaaggacaacatggaatcaagtttcaataatttttagattgtacataaataaagagaccagactaatacaactgtataagctctaaacattcaattgccaaaaaacattaaca

SEQ ID NO: 39 - DNA, 862 bp, artificial sequence
  cgHHT2 promoter
tgttattgattatttatttatttgttgttattttattgtatttttttttaaaaagctactaatacacaacaaaatgtaaaccacaaactctcccaaacagaacgaacaccctggctttatatacaaaatttcttatcccgaaatgattgtcaacaagataaacaaaaagaagccaccacaggaaccctaccccccctctctcccacccccatcgtgtcttcccttccaccttccacccttcacctggccatagtaacttgaggtgtgcacactccagaacggtcgacccactacggatctgaattccagtgcgaagttgcgggaacggatttattatgaccaggcggctactaagaagctcaaaccatcaaaaacgcgcgaagaaactggtctacccgtagcaagttactattctccaaattagtgtttgtcgtgtgccgtgcgtcgtgggtagaccgagattttcgcgcgcctcaggtggttcgagggcaaataaccatagttaagccgttcccattctttctcacatgtacgtgccggacgcttcgatgaaatttttgtgaatcgtttcgttgcttctgtgaccacaaaacagcctggagaggaggcgttgcgtaaggggggggggcctgatgagaaacagatgacacaacgcagaactggctgcagtgcaaaggcatttcctctgccacacacactaccataaccagtctcggtattaactggctcaacaaacgataagagccaaagaggaagaaaaaaaggtatttaaaggatgtatttctttatctcctgattagatttcttacttgtacagaaactgtaactaaaaaaaacaacaacaaaacacaacacatacata

SEQ ID NO: 40 - DNA, 999 bp, artificial sequence
  cgMET3 promoter
caagtacaaattatgtaatatggttataattggcttatatgtatggaggtagggaactgcaggttattgttctgttaaactaccagtaatgcaagacggctaagtcatatgactgccactttgtcaatcctgaagaaaatgacaatagaaagtgatataatacacgtgatatgcgtaggatgggacccaaagatagccacatcacatgatcttcaaaaaccccgtcaaaaaggaataaatcgaaggaaaaaaaatgccacgtgactttgatagtcaaaatcaggttatattactatacgatcccccacacataacctttaccacacaccgcacgggctggacttatcctattagaaagcggtgcagccagggccaagaaaacgcgaacgacgcagaaaaaacgtcaagcaaaaaaactgtggtgttatttaaagcatacaatttcctgcctccctttaatgctacgggggatctagcaaatgggaaaatatcacatgactttggctagaaggggtgggaaaacagatattttagtcacattgtttgatttcacgtactacacgatacactacactacacaatacactgcaatacactacactgtatggttccctccggccctggatatgctagcgaaggatcccaagcccatcgaggaaatcattcaagagcatctgcaggtctcaaatacactaagtccaaacacaaacagcactgagctaatccaagacaccaattgcatgccttctcaattagaacagtttattacacagcttataactgtgcatcttgccattcttttcgagatagccatttgcatttgattaacatgcttagctcgttctcaagccacagtaaaatggattgccttttgagtttccatcgttgtataaataggcactcattcctatagccttgctctttggtcttgtctctgtaatggtaatagctgtaagtcaggatataacactccagaaaagaaacacctaacaatctaga

SEQ ID NO: 41 - DNA, 253 bp, artificial sequence
  CYC1 terminator
tcatgtaattagttatgtcacgcttacattcactgtsdgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgc

SEQ ID NO: 42 - DNA, 185 bp, artificial sequence
  ADH1 terminator
gcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagtatgaggtcgctcttattgaccacacctctac

SEQ ID NO: 43 - DNA, 419 bp, artificial sequence
  SV40 promoter
gcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 44 - DNA, 1278 bp, artificial sequence
  Chicken beta-actin promoter
agtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagcccccaa

SEQ ID NO: 45 - DNA, 743 bp, artificial sequence
  CMV promoter
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtca

SEQ ID NO: 46 - DNA, 242 bp, artificial sequence
  Human U6 promoter
agggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacga

SEQ ID NO: 47 - DNA, 68 bp, artificial sequence
  Bovine Growth Hormone signal sequence
aataaaatgaggaaattgcatcgcattgtctgagtaggtgtcattctattctggggggtggggtgggg

SEQ ID NO: 48 - DNA, 196 bp, artificial sequence
  Human Growth Hormone signal sequence
ctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctggaggggaccggtgatgaggggat

SEQ ID NO: 49 - DNA, 240 bp, artificial sequence
  SV40 signal sequence
atctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 50 - DNA, 144 bp, artificial sequence
  Rabbit Beta-Globin signal sequence
accccttcactgtaggacagagcttctagcaagaagctttatccctcaaataataatgaaaataataaaactactctaagaaattatttgtgatggtattgagtttattttccttgtacttttaaatatatggtcctcaaggga

SEQ ID NO: 51 - DNA, 49 bp, artificial sequence
  Synthetic poly A signal sequence
aataaaatatctttattttcattacatctgtgtgttggttttttgtgtg